(19) United States
(12) Patent Application Publication
(10) Pub. No.: US 2010/0234402 A1
(43) Pub. Date: Sep. 16, 2010

(54) METHODS AND COMPOSITIONS FOR TREATING SPINAL MUSCULAR ATROPHY

(76) Inventors: Gideon Dreyfuss, Wynnewood, PA (US); Lili Wan, Westfield, NJ (US)

Correspondence Address: Pearl Cohen Zedek Latzer, LLP, 1500 Broadway, 12th Floor, New York, NY, 10036, US

(21) Appl. No.: 12/602,423

(22) PCT Filed: Jun. 2, 2008
(86) PCT No.: PCT/US08/06944

§ 371 Date: Jun. 1, 2010

Related U.S. Application Data
(60) Provisional application No. 60/924,782, filed on May 31, 2007.

(51) Int. Cl. A61K 31/522; A61K 31/375; A61K 31/355; A61K 31/395; A61K 31/122; A61K 31/385; A61K 31/197; A61K 31/4025; A61P 25/00; A61P 21/00

(52) U.S. Cl. 514/263.33; 514/474; 514/458; 514/359; 514/678; 514/440; 514/562; 514/422

(57) ABSTRACT

This invention provides methods of treating and abrogating spinal muscular atrophy by administering an antioxidant. Additionally, the invention provides a method of protecting the SMN protein by administering an antioxidant.

### FIELD OF INVENTION

This invention provides: methods and compositions comprising antioxidants for the treatment of spinal muscular atrophy.

### BACKGROUND OF THE INVENTION

Spinal muscular atrophy (SMA) is one of the most common autosomal recessive diseases, affecting approximately 1 in 6,000 to 10,000 live births, and is the leading hereditary cause of infant mortality. SMA is a neurodegenerative disease of motor neurons that results in progressive muscle weakness and death from respiratory failure, and is caused by mutations in the survival of motor neurons (SMN) gene.

In order to be diagnosed with Spinal muscular atrophy, symptoms need to be present. In most cases a diagnosis can be made by the SMN gene test, which determines whether there is at least one copy of the SMN1 gene by looking for its unique sequences (that distinguish it from the almost identical SMN2) in exons 7 and 8. In some cases, when the SMN gene test is not possible or does not show any abnormality, other tests such as an electromyography (EMG) or muscle biopsy may be indicated.

The region of chromosome 5 that contains the SMN gene has a large duplication. A large sequence that contains several genes occurs twice in adjacent segments. There are thus two copies of the gene, SMN1 and SMN2. The SMN2 gene has an additional mutation that makes it less efficient in making protein, though it does so in a low level. SMA is caused by loss of the SMN1 gene from both chromosomes. The severity of SMA, ranging from SMA1 to SMA3, is partly related to how well the remaining SMN2 genes can make up for the loss of SMN1. Often there are additional copies of SMN2, and an increasing number of SMN2 copies cause less severe disease.

Infantile SMA—Type 1 or Werdnig-Hoffmann disease (generally 0-6 months): SMA type 1 is the most severe, and manifests in the first year of life with the inability to ever maintain an independent sitting position. Intermediate SMA—Type 2 (generally 7-18 months): Type 2 SMA describes those children who are never able to stand and walk, but who are able to maintain a sitting position at least some time in their life. The onset of weakness is usually recognized some time between 6 and 18 months. Juvenile SMA—Type 3 or Kugelberg-Welander disease (generally >18 months): SMA type 3 describes those who are able to walk at some time. Adult SMA—Type 4: Weakness usually begins in late adolescence in tongue, hands, or feet then progresses to other areas of the body. The course of disease is much slower and has little or no impact on life expectancy.

The SMN complex functions in the biogenesis of snRNPs (small nuclear ribonucleoproteins), the major components of the spliceosome. The spliceosome is the pre-mRNA splicing machinery that produces mRNAs from pre-mRNAs in eukaryotic cells.

### SUMMARY OF THE INVENTION

This invention provides, in one embodiment, a method of treating a spinal muscular atrophy in a subject, comprising administering to a subject a compound which inhibits SMN protein oxidation.

In another embodiment, the present invention provides a method of abrogating spinal muscular atrophy in a subject, comprising a subject a compound which inhibits SMN protein oxidation.

In another embodiment, the present invention provides a method of preventing a spinal muscular atrophy in a subject, comprising administering to a subject a compound which inhibits SMN protein oxidation.

In another embodiment, the present invention provides a method of protecting an SMN protein in a subject, comprising administering to a subject a compound which inhibits SMN protein oxidation.

In another embodiment, the present invention provides a method of protecting the generation of a spliceosome in a subject, comprising administering to a subject a compound which inhibits SMN protein oxidation.

In another embodiment, the present invention provides a method of protecting the generation of small nuclear ribonucleoproteins (snRNPs) in a subject, comprising administering to a subject a compound which inhibits SMN protein oxidation.

In another embodiment, the present invention provides a method of treating a disease mediated by a deficient spliceosome in a subject, comprising administering to a subject a compound which inhibits SMN protein oxidation.

In another embodiment, the present invention provides a method of protecting an SMN protein in a subject at risk of developing SMA, comprising the step of administering to said subject a compound which inhibits SMN protein oxidation.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 Depicts an experimental scheme of a high throughput SMN complex activity assay for the detection of in vitro-assembled snRNPs in a 384-well microplate format.

FIG. 2 shows high throughput screening for small molecule modulators of the activity of the SMN complex in snRNP assembly (A) Scatter plot of a representative 384-well microplate from a chemical library screening. Each dot represents the signal from one well containing a standard assembly reaction mixture in the presence of individual compounds at 20 μM final concentration. The green box indicates reactions lacking cell extracts, representing the non-specific background of the assay. The red circles indicate wells containing compounds that significantly decreased the activity of the SMN complex. (B) High throughput screening and selection of potential inhibitors of the activity of the SMN complex. ˜5,000 compounds were screened in triplicate at 20 μM final concentration. As shown, 22 compounds were selected as potential inhibitors based on the criteria that they inhibited the activity of the SMN complex by more than 3 times the standard deviation (>3SD) of the assay, as indicated by the red dotted line and shaded area. The compounds are labeled according to their designations in the library. The error bars represent SDs from triplicate samples. Validation of potential SMN complex inhibitors by gel mobility shift assay using [³²P]UTP-labeled U1 snRNA mixed with HeLa total cell extracts in the presence of 20 or 100 μM compound, or cycloheximide or DMSO controls. (C) represents a gel mobility shift assay for the confirmation of potential SMN complex inhibitors. Gel mobility shift assay [³²P]UTP-labeled U1 snRNA was mixed with HeLa total cell extracts in the presence of 20 or 100 M compound, or cycloheximide or DMSO controls for in vitro snRNP assembly and analysis by native polyacrylamide gel electrophoresis. FIG. 3C represents a bar graph showing the assessment of selectivity of potential SMN complex inhibitors by in vitro transcription and translation assay. Reactions were set up using luciferase DNA as a reporter in the presence of either 20 M compound or DMSO control. Luciferase activities of the in vitro produced proteins were measured and compared to that of DMSO control (100% activity). The error bars represent SDs from triplicate samples. (D) Assessment of selectivity of potential SMN complex inhibitors by in vitro transcription and translation assay. Reactions were set up using luciferase DNA as a reporter in the presence of either 20 μM compound or DMSO control. Luciferase activities of the in vitro produced proteins were measured and compared to that of DMSO control (100% activity). The SD of this assay is 3.5%. The error bars represent SDs from measurements of each compound in triplicate and the red dotted line indicates 3SD.

FIG. 3 shows that b-lapachone potently and selectively inhibits the SMN complex-mediated snRNP assembly in vitro and in cells (A) Chemical structure of β-lapachone. (B) Concentration-dependent inhibition of the activity of the SMN complex by b-lapachone in cells. HeLa cells were treated with various concentrations of β-lapachone or with DMSO (control) for 1 hour. The activity of the SMN complex in extracts from treated cells was measured using magnetic beads snRNP assembly assay and compared to that of DMSO-treated control cells (100% activity). IC₅₀ was calculated from the dose-response graph. The error bars represent SDs from 3 independent measurements. (C) β-lapachone selectively inhibits SMN complex-mediated Sm core assembly. Assembly reactions were performed using either cell extracts or purified native 39 snRNP proteins lacking the SMN complex (Sm proteins). Both samples were adjusted to contain a similar amount of Sm proteins. Magnetic beads snRNP assembly assay were carried out with U4 or control U4DSm snRNA in the presence of either 20 or 100 μM β-lapachone or DMSO control. Sm core assembly on U4 snRNA in the presence of DMSO was considered 100% activity. The error bars represent SDs from 3 independent measurements.

FIG. 4 shows SMN protein is oxidized to form intermolecular disulfide bonds upon β-lapachone treatment (A) Indirect immunofluorescence staining of SMN (2B1; green) and snRNPs (Y12; red) in HeLa PV cells treated for 3 hours with 5 μM β-lapachone or DMSO control. (B) HeLa total cell extracts prepared from cells treated for 3 hours with 5 μM β-lapachone or DMSO control were resolved by SDS-PAGE and analyzed by quantitative Western blotting, using JBP1 and Magoh as loading controls. The extracts were prepared and mixed with sample buffer without reducing agent. The membrane was cut into strips for the probing of each protein at the corresponding molecular mass. (C) β-lapachone causes intermolecular disulfide crosslinking of SMN. Total cell extracts from HeLa cells stably expressing Flag-Gemin2 (Yong et al., 2002) were used for in vitro assembly reactions in the presence of either 100 μM β-lapachone or DMSO control. The SMN complex was isolated by anti-Flag immunoprecipitation, mixed with sample buffer without (−DTT) or with (+DTT) 40 reducing agent and resolved by SDS-PAGE. Western blot analysis was performed on the entire membrane with anti-SMN antibody 62E7. The molecular mass markers in kDa are indicated on the left. “redSMN” indicates monomer SMN migrating at normal molecular mass and “oxSMN” indicates disulfide-crosslinked SMN upon oxidation.

FIG. 5 shows ROS reagents inhibit the activity of the SMN complex in vitro and in cells (A) Effect of H2O2 on the activity of the SMN complex in vitro. Magnetic beads snRNP assembly assay was carried out in the presence of increasing amounts of H2O2. IC50 was calculated from the dose-response curve. The error bars represent SDs from 3 independent measurements. (B) Effect of menadione on the activity of the SMN complex in vitro. The same experimental procedure was carried out as in (A), except that menadione was used instead. (C) Dose-dependent effect of menadione on the activity of the SMN complex in cells. HeLa cells were treated with menadione at the indicated concentrations or with DMSO control for 1 hour. The activity of the SMN complex in extracts from various treated cells was measured in comparison to DMSO control cell extract (100% activity) by magnetic beads snRNP assembly assay. The error bars represent SDs from 3 independent measurements. (D) Extracts from (C) mixed with sample buffer without (−DTT) or with (+DTT) reducing agent were resolved by SDS-PAGE and analyzed by Western blot of the entire membrane with anti-SMN antibody 62E7. The molecular mass markers in 41 kDa are indicated on the left. “redSMN” indicates monomer SMN migrating at normal molecular mass and “oxSMN” indicates disulfide-crosslinked SMN upon oxidation. (E) β-lapachone and menadione generate ROS in live cells. HeLa cells were incubated with ROS indicator dye H2DCFDA (10 μM) or without dye as a control for 30 minutes, and then treated with the compounds at the indicated concentrations or DMSO as a control. Fluorescence images were acquired 30 minutes after the treatment.

FIG. 6 shows DTT prevents the inhibition of the activity of the SMN complex by β-lapachone the inhibition of the activity of the SMN complex by β-lapachone is reversible by the reducing agent DTT and the extent of SMN oxidative crosslinking correlates with the decrease of SMN complex activity. (A) Cell extracts treated with 20 μM β-lapachone, or 20 μM β-lapachone together with 20 μM DTT, or DMSO control were analyzed by non-reducing Western blot. The relative levels of monomer SMN (“redSMN”) were calculated as the percentage of that in DMSO control and shown by the blue bar. Assembly activities of the SMN complex were measured by magnetic beads snRNP assembly assay using the same set of treated extracts and shown by the red bar. The error bar represents SDs from 3 independent experiments. (B) The sulfhydryl modifying reagent iodoacetamide inhibits the activity of the SMN complex. In vitro assembly assays were performed in the presence of various concentrations of iodoacetamide. The activity of the SMN complex in the absence of iodoacetamide was set as 100% activity, and the relative activity at each iodoacetamide concentration was calculated in comparison and graphed. The error bars represent SDs from triplicate samples.

FIG. 7. SMN shows that a protein is oxidized to form intermolecular disulfide bonds upon β-lapachone treatment. (A) Indirect immunofluorescence staining of SMN (2B1; gray) and snRNPs (Y12; bright spots in nuclei) in HeLa PV cells treated for 3 hours with 5 μM β-lapachone or DMSO (B) HeLa total cell extracts prepared from cells treated for 3 hours with 5 μM β-lapachone or DMSO control were resolved by SDS-PAGE and analyzed by quantitative Western blotting, using JBP1 and Magoh as loading controls. Prior to loading onto the gel, the extracts were mixed with sample buffer without reducing agent. The membrane was cut into strips for the probing of each protein at the corresponding molecular mass. (C) The signal intensity of the SMN protein bands in panel B was quantitated using a Li-Cor Odyssey infrared imaging system. The relative level of SMN protein in β-lapachone-treated cells was calculated as the percentage of that in DMSO treated control cells. The error bar represents SDs from 4 independent experiments. (D) β-lapachone causes intermolecular disulfide crosslinking of SMN. Total cell extracts from HeLa cells stably expressing Flag-Gemin2 were used for in vitro assembly reactions in the presence of either 100 μM β-lapachone or DMSO control. The SMN complex was isolated by anti-Flag immunoprecipitation, mixed with sample buffer without (−DTT) or with (+DTT) reducing agent and resolved by SDS-PAGE. Western blot analysis was performed on the entire membrane with anti-SMN antibody 62E7. The molecular mass markers in kDa are indicated on the left.

### DETAILED DESCRIPTION OF THE INVENTION

In one embodiment, the present invention provides a method of treating a spinal muscular atrophy in a subject, comprising administering to a subject a compound which inhibits SMN protein oxidation. In another embodiment, the present invention provides a method of abrogating spinal muscular atrophy in a subject, comprising a subject a compound which inhibits SMN protein oxidation. In another embodiment, the present invention provides a method of preventing a spinal muscular atrophy in a subject, comprising administering to a subject a compound which inhibits SMN protein oxidation.

In another embodiment, a compound which inhibits SMN protein oxidation is a compound of the present invention. In another embodiment, a compound which inhibits SMN protein oxidation is an antioxidant. In another embodiment, a compound which inhibits SMN protein oxidation is a compound which reverses SMN oxidation. In another embodiment, a compound which reverses SMN oxidation is a reducing agent.

In another embodiment, a compound which inhibits SMN protein oxidation is a compound which boosts subject's intrinsic antioxidant mechanisms. In another embodiment, subject's intrinsic antioxidant mechanisms are antioxidant enzymes. In another embodiment, a compound which inhibits SMN protein oxidation is a compound which boosts catalase enzymatic activity. In another embodiment, a compound which inhibits SMN protein oxidation is a compound which boosts glutathione enzymatic activity. In another embodiment, a compound which inhibits SMN protein oxidation is a compound which boosts peroxidase enzymatic activity. In another embodiment, a compound which inhibits SMN protein oxidation is a compound which boosts superoxide dismutase (SOD) enzymatic activity.

In one embodiment, the present invention provides a method of treating spinal muscular atrophy (SMA), comprising administering an antioxidant.

In another embodiment, the present invention provides a method of abrogating spinal muscular atrophy, comprising administering an antioxidant.

In another embodiment, the present invention provides a method of preventing spinal muscular atrophy, comprising administering an antioxidant.

In another embodiment, the present invention provides a method of protecting an SMN protein in a subject, comprising administering to a subject a compound which inhibits SMN protein oxidation. In another embodiment, the present invention provides a method of protecting an SMN protein, comprising administering an antioxidant. In another embodiment, the present invention provides a method of protecting an SMN protein from oxidative stress, comprising administering an antioxidant. In another embodiment, the present invention provides a method of protecting an SMN protein from ROS, comprising administering an antioxidant. In some embodiments, protecting an SMN protein from oxidative stress provides treatment for spinal muscular atrophy. In another embodiment, protecting an SMN protein from oxidative stress abrogates spinal muscular atrophy. In another embodiment, protecting an SMN protein from oxidative stress inhibits spinal muscular atrophy. In another embodiment, protecting an SMN protein from oxidative stress prevents spinal muscular atrophy. In another embodiment, protecting an SMN protein from oxidative stress is achieved by administering an antioxidant thus preventing oxidative damage.

In another embodiment, “oxidative stress” is co μM only used in reference to biological systems as a means to characterize the total burden of potentially harmful reactive oxygen species that are present in tissues as a consequence of routine cellular oxidative metabolism of both endogenous and exogenous compounds. In another embodiment, many of the chemical reactions that contribute to oxidative-stress are not oxidative in nature. In another embodiment, oxidative stress is the production of free radical species in vivo, though, a number of species associated with oxidative stresses either are not radicals or radical species that are not inherently deleterious.

In another embodiment, compounds that cause oxidation inactivate the SMN complex. In another embodiment, oxidants inactivate the SMN complex. In another embodiment, reactive oxygen species (ROS) generators inactivate the SMN complex. In another embodiment, compounds that cause oxidation inactivate the SMN complex in a dose-dependent manner both in vitro and in vivo. In another embodiment, oxidants cause the SMN protein to form intermolecular disulfide cross-links. In another embodiment, oxidants cause components of the SMN complex to form intermolecular disulfide cross-links.

In another embodiment, the present invention provides a compound that reverses inactivation of the SMN complex. In another embodiment, the present invention provides a compound that reverses inactivation of the SMN complex due to oxidative stress.

In another embodiment, a method of the present invention utilizes a compound that reverses oxidation of an SMN complex. In another embodiment, a method of the present invention utilizes a compound that converts an SMN complex from an oxidized state to a reduced state. In another embodiment, the present invention provides a compound that reverses oxidation of an SMN complex by enhancing recovery of oxidized SMN complex to the active reduced state. In another embodiment, the present invention provides a method of enhancing recovery of oxidized SMN complex to the active reduced state, comprising the step of administering a compound that induces protein disulfide isomerase (PDI). In another embodiment, PDI catalyzes the formation and breakage of disulfide bonds between cysteine residues within proteins as they fold. In another embodiment, PDI catalyzes the formation and breakage of disulfide bonds between cysteine residues within SMN complex proteins. In another embodiment, PDI allows SMN complex proteins to correct arrangement of disulfide bonds. In another embodiment, reduced (dithiol) form of PDI is able to catalyze a reduction of mispaired thiol residues of an SMN complex, acting as an isomerase. In another embodiment, PDI is capable of catalyzing the post-translational disulfide exchange. In another embodiment, exchange reactions occur intramolecularly, leading to the rearrangement of disulphide bonds in an SMN complex protein.

In another embodiment, a compound that reverses SMN complex from oxidized state to reduced state is a reducing agent.

In another embodiment, the present inventions provide methods of protecting SMN complex in a cell, comprising administration a compound of the present invention to a cell. In another embodiment, the present inventions provide methods of preventing SMN complex oxidation in a cell, comprising administration a compound of the present invention to a cell. In another embodiment, administration of a compound of the present invention to a cell comprises administering a compound of the present invention to a subject. In another embodiment, administration of a compound of the present invention to a cell comprises administering a compound of the present to a cell culture media comprising a cell.

In another embodiment, the present invention provides a method comprising prophylactic treatment of a disease characterized by oxidation and thus inactivation of the SMN complex. In another embodiment, the present invention provides a method comprising prophylactic treatment of a disease neurodegenerative disease characterized by oxidation and thus inactivation of the SMN complex. In another embodiment, the present invention provides a method comprising prophylactic treatment of SMA. In another embodiment, the present invention provides a method of treating a neurodegenerative disease, comprising the step of preventing oxidation of an SMN complex with a compound of the present invention. In another embodiment, the present invention provides a method of treating a neurodegenerative disease, comprising the step of preventing oxidation of an SMN complex with an antioxidant.

In another embodiment, a neurodegenerative disease of the present invention comprises SMA, SBMA, ALS, Alzheimer's disease, Parkinson's disease, or Huntington's disease.

In another embodiment, the present invention provides a method comprising prophylactic treatment of a disease characterized by oxidation and thus inactivation of the SMN complex comprising administering an antioxidant of the present invention. In another embodiment, the present invention provides a method comprising prophylactic treatment of a disease characterized by oxidation and thus inactivation of the SMN complex comprising administering a compound that boosts the subject's intrinsic antioxidant defense.

In another embodiment, a compound of the present invention boosts the activities of enzymes involved in the detoxification of reactive oxygen species. In another embodiment, the compound of the present invention boosts the activity of catalase. In another embodiment, the compound of the present invention boosts the activity of glutathione reductase. In another embodiment, the compound of the present invention boosts the activity of peroxidase. In another embodiment, the compound of the present invention boosts the activity of superoxide dismutase (SOD).

In another embodiment, the present invention provides that glutathione is the brain's master antioxidant and plays an important protective role in the brain. In another embodiment, a compound of the present invention protects glutathione metabolism. In another embodiment, a compound of the present protects glutathione metabolism. In another embodiment, a compound of the present invention protects the subject's intrinsic antioxidant defenses. In another embodiment, the compound of the present invention which protects the subject's intrinsic antioxidant defenses. In another embodiment, the compound of the present invention protects the SMN complex from oxidants that inactivate the SMN complex.

In another embodiment, compounds that cause toxicity to certain brain cells decrease cerebral glutathione (GSH), making the cells more vulnerable to reactive oxygen species (ROS). In another embodiment, over-expression of the glutathione peroxidase (GPX) enzyme potently decreases cell death from brain injury. In another embodiment, the present invention provides a method comprising administering a GPX activator and/or booster.

In another embodiment, the methods of the present invention comprise administering glutathione precursors. In another embodiment, the methods of the present invention comprise administering N-acetyl-cysteine (NAC) which is a precursor of glutathione. In another embodiment, the methods of the present invention comprise administering intravenous glutathione therapy. In another embodiment, the methods of the present invention comprise administering supplements effective in boosting intracellular levels of glutathione. In another embodiment, the methods of the present invention comprise administering a glutathione precursor in combination with a protocol that lowers homocysteine levels comprising B12 and folate. In another embodiment, the methods of the present invention comprise administering NAC in combination with a protocol that lowers homocysteine levels comprising B12 and folate.

In another embodiment, the methods of the present invention comprise administering curcumin (turmeric). In another embodiment, curcumin comprises neuroprotective effects. In another embodiment, curcumin induces the enzyme, hemeoxygenase-1 (HO-1), which protects neurons exposed to oxidant stress. In another embodiment, curcumin increases the expression of HO-1 protein. In another embodiment, curcumin increases the expression of glutathione S-transferase.

In another embodiment, the methods of the present invention comprise administering Ebselen. In another embodiment, Ebselen is a glutathione peroxidase mimic. In another embodiment, Ebselen is a potent synthetic antioxidant. In another embodiment, Ebselen is a neuroprotective agent. In another embodiment, Ebselen is an inhibitor of free-radical induced apoptosis.

In another embodiment, the methods of the present invention comprise administering undenatured Whey protein. In another embodiment, undenatured Whey protein provides glutathione precursors. In another embodiment, undenatured Whey protein raises intracellular glutathione levels.

In another embodiment, the methods of the present invention comprise administering anti-ROS compounds. In another embodiment, the methods of the present invention comprise administering ROS-scavenging compounds. In another embodiment, ROS comprises hydroxyl radicals, peroxynitrite, hypochlorous acid and hydrogen peroxide. In another embodiment, an antioxidant that scavenges, or reacts with, superoxide is a superoxide dismutase mimic (SOD-mimic), superoxide scavenger, or superoxide dismutase mimetic (SOD-mimetic).

In another embodiment, a ROS scavenger compound comprises an alkenyl group; aryl group; substituted aryl group, where the aryl group is substituted with, for example, —OH, —NH2, or —NHCHO; sulfhydryl (in a protected form) or dithiol in oxidized or reduced form; or a group that is, or is capable of being converted in vivo into, a sulfhydryl in its oxidized or reduced form. In another embodiment, the compound of the present invention is a bi-functional anti-inflammatory SOD-mimetic. In another embodiment, the SOD-mimetic compound comprises a nitroxide free radical group, or a dithiol structure in its oxidized form, such as lipoic acid analog. In another embodiment, the compounds as described herein may comprise more than one ROS scavenger component.

In another embodiment, the methods of the present invention comprise administering Lipoic acid (LA). In another embodiment, LAis an essential cofactor in mitochondrial-keto acid dehydrogenase complexes.

In another embodiment, SMN is in a complex with Gemins. In another embodiment, SMN-Gemins complex is essential for the biogenesis of small nuclear ribonucleoproteins (snRNPs). In another embodiment, snRNPs are the major constituents of the spliceosome.

In another embodiment, the present invention provides a method of protecting the generation of small nuclear ribonucleoproteins (snRNPs) in a subject, comprising administering to a subject a compound which inhibits SMN protein oxidation. In another embodiment, damaged SMN complex inhibits the generation of snRNPs. In another embodiment, SMN is damaged by oxidative damage. In another embodiment, SMN is damaged by ROS. In another embodiment, damaged SMN complex inhibits the generation of snRNPs which in turn inhibit the generation of the spliceosome.

In another embodiment, the present invention provides that the level of snRNP assembly can be estimated by autoradiography on a phosphorimager. In another embodiment, the present invention provides that while the ³²P-labeled RNA bands obtained after immunoprecipitation with anti-Sm antibodies are suitable to estimate assembled Sm cores. In another embodiment, the present invention provides a quantitative measurement of Sm core formation. In another embodiment, the present invention that snRNAs are prepared by in vitro transcription in the presence of biotin-UTP. In another embodiment, the present invention provides that following in vitro assembly reactions with the biotin-labeled RNAs, immunoprecipitations of the Sm cores are carried out under stringent conditions, including high salt (500 mM NaCl) and heparin (2 mg/ml). In another embodiment, the present invention provides that the immunoprecipitations are carried out with Y12 antibodies immobilized on magnetic beads in a multi-well plate format, which allows automatic cycles of washing and mixing of the beads on a robotic manifold. In another embodiment, the present invention provides that horseradish peroxidase-conjugated avidin, which binds tightly to biotin, is used to recognize the biotinylated RNAs in the Y12 immunoprecipitated Sm cores. In another embodiment, the present invention provides that this step serves to amplify the signals for the luminescence measurement of the horseradish peroxidase activity on an automatic plate reader.

In another embodiment, the present invention provides that the SMN complex is required for snRNP assembly. In another embodiment, the present invention provides that complete removal or inhibition of the SMN complex results in the inhibition of Sm core assembly in vitro. In another embodiment, the present invention provides that nearly complete removal or inhibition of the SMN complex results in the inhibition of Sm core assembly in vitro.

In another embodiment, the present invention provides that SMA results from a reduction in the amount of the full-length SMN protein. In another embodiment, the present invention provides that SMN expression is more reduced in the severe form (type I) than the mild form (type III) of the disease, demonstrating a direct correlation between the degree of reduction of SMN protein levels in SMA patients and the severity of their clinical phenotypes. In another embodiment, the present invention provides that snRNP assembly is impaired in cells of SMA patients. In another embodiment, the present invention provides that provides that impairment of snRNP assembly reduces capacity to assemble Sm cores.

In another embodiment, the present invention provides a method of protecting the generation of a spliceosome in a subject, comprising administering to a subject a compound which inhibits SMN protein oxidation. In another embodiment, the present invention provides a method of protecting the spliceosome, comprising administering an antioxidant. In another embodiment, the present invention provides a method of protecting the spliceosome from oxidative stress, comprising administering an antioxidant. In another embodiment, the present invention provides a method of protecting the spliceosome from ROS, comprising administering an antioxidant. In some embodiments, protecting the spliceosome from oxidative stress provides treatment for spinal muscular atrophy. In another embodiment, protecting the spliceosome from oxidative stress abrogates spinal muscular atrophy. In another embodiment, protecting the spliceosome from oxidative stress inhibits spinal muscular atrophy. In another embodiment, protecting the spliceosome from oxidative stress prevents spinal muscular atrophy. In another embodiment, protecting the spliceosome from oxidative stress is achieved by administering an antioxidant thus preventing oxidative damage.

In another embodiment, the present invention provides a method of protecting small nuclear ribonucleoproteins (snRNPs), comprising administering an antioxidant. In another embodiment, the present invention provides a method of protecting snRNPs from oxidative stress, comprising administering an antioxidant. In another embodiment, the present invention provides a method of protecting snRNPs from ROS, comprising administering an antioxidant. In some embodiments, protecting snRNPs from oxidative stress provides treatment for spinal muscular atrophy. In another embodiment, protecting snRNPs from oxidative stress abrogates spinal muscular atrophy. In another embodiment, protecting snRNPs from oxidative stress inhibits spinal muscular atrophy. In another embodiment, protecting snRNPs from oxidative stress prevents spinal muscular atrophy. In another embodiment, protecting snRNPs from oxidative stress is achieved by administering an antioxidant thus preventing oxidative damage.

In another embodiment, Spinal Muscular Atrophy (SMA) is a motor neuron disease. In another embodiment, motor neurons are nerve cells in the spinal cord which send out nerve fibers to muscles throughout the body. Each possibility represents a separate embodiment of the present invention.

In another embodiment, defective SMN causes nerve cells atrophy, shrink and die, resulting in muscle weakness. In another embodiment, SMA affects motor neurons affecting the voluntary muscles that are used for activities such as crawling, walking, head and neck control, and swallowing. In another embodiment, SMA affects muscles throughout the body. In another embodiment, SMA severely affects proximal muscles. In some embodiments, proximal muscles comprise those closest to the trunk of one's body—i.e. shoulders, hips, and back. In another embodiment, SMA affects feeding and swallowing. In another embodiment, the respiratory muscles are affected. In another embodiment, SMA causes increased tendency for pneumonia and other lung problems. Each possibility represents a separate embodiment of the present invention.

In another embodiment, SMA has a missing or mutated gene (SMN1, or survival motor neuron 1) that produces a protein in the body called Survival Motor Neuron (SMN) protein.

In another embodiment, SMA is Type I SMA or Werdnig-Hoffmann Disease. In another embodiment, diagnosis of children with this type is usually made before 6 months of age. In another embodiment, diagnosis of children with this type is usually made before 3 months of age. In another embodiment, a child with Type I is never able to lift his/her head or accomplish the normal motor skills expected early on in infancy. In another embodiment, a child with Type I does not have the ability to sit up unsupported. In another embodiment, a child with Type I will have difficulties in swallowing and feeding. In another embodiment, Type I causes tongue atrophy, and rippling movements or fine tremors, also called fasciculations. In another embodiment, a child with Type I will suffer from weakness of the intercostal muscles, and the chest is often smaller than usual. In another embodiment, Type I causes the chest to concave. In another embodiment, the lungs may not fully develop in a child with Type I.

In another embodiment, SMA is Type II SMA. In another embodiment, diagnosis of children with type II SMA is usually made before 2 years of age. In another embodiment, diagnosis of children with type II SMA is usually made before 15 months of age. In another embodiment, children with this type may sit unsupported when placed in a seated position, although they are often unable to come to a sitting position without assistance. In another embodiment, children with this type may have difficulty eating enough food by mouth to maintain their weight and grow, and a feeding tube may become necessary. In another embodiment, children with this type frequently have tongue fasciculations and manifest a fine tremor in the outstretched fingers. In another embodiment, children with this type frequently have weak intercostals muscles and are diaphragmatic breathers. In another embodiment, children with this type frequently have difficulty coughing and may have difficulty taking deep enough breaths while they sleep to maintain normal oxygen levels and carbon dioxide levels. In another embodiment, children with this type frequently have scoliosis is almost uniformly present as these children grow, resulting in need for spinal surgery or bracing at some point in their clinical course. In another embodiment, children with this type frequently have decreased bone density can result in an increased susceptibility to fractures.

In another embodiment, SMA is Type III SMA or Kugelberg-Welander or Juvenile Spinal Muscular Atrophy Disease. In another embodiment, diagnosis of children with type II SMA is usually made from around a year of age or even as late as adolescence, although diagnosis prior to age 3 years is typical. In another embodiment, patients with this type can stand alone and walk, but may show difficulty with walking at some point in their clinical course. In another embodiment, patients with this type can walk but may fall more frequently, have difficulty in getting up from sitting on the floor or a bent over position, and may be unable to run. In another embodiment, With Type III, a fine tremor can be seen in the outstretched fingers but tongue fasciculations are seldom seen. In another embodiment, with Type III, Feeding or swallowing difficulties in childhood are very uncommon. In another embodiment, Type III individuals can sometimes lose the ability to walk later in childhood, adolescence, or even adulthood, often in association with growth spurts or illness.

In another embodiment, SMA is Type IV SMA or adult onset. In another embodiment, Type IV symptoms typically begin after age 35. In another embodiment, Type IV is typically characterized by insidious onset and very slow progression. In another embodiment, patients with SMA typically lose function over time.

In another embodiment, SMA is diagnosed through a blood test. In another embodiment, a blood test is directed to presence or absence of the SMN1 gene, in conjunction with a suggestive history and physical examination. In another embodiment, SMN1 protein is missing. In another embodiment, SMN1 protein is mutated. In another embodiment, the numbers of copies of SMN2, a near identical backup copy of the SMN1 gene, are related to the severity of the disease, but do not reliably predict a specific SMA type in a given individual. In some embodiments, SMA type is generally determined from the clinical examination evaluating the child's degree of weakness and ability to achieve major motor milestones such as sitting independently or walking. In another embodiment, SMA is further diagnosed through muscle biopsy or EMG (electromyography) testing.

In another embodiment, SMN1 gene encodes the SMN Protein. In another embodiment, the absence of this SMN1 gene that causes Spinal Muscular Atrophy. In another embodiment, SMN1 gene encodes the SMN Protein. In another embodiment, the defect of this SMN1 gene that causes Spinal Muscular Atrophy. In another embodiment, another form of this gene is called SMN2. In another embodiment, SMN2 gene is similar to SMN1, but does not produce as much protein, or the right kind of protein, as the SMN1 gene. In another embodiment, determination of prognosis is the number of copies of the SMN2 gene. In another embodiment, the greater the number of SMN2 copies, the more SMN protein is produced and the greater likelihood that more motor neurons remain healthy and productive. In another embodiment, individuals with only 1 or 2 copies of the SMN2 gene will typically have the most severe expressions of SMA. In another embodiment, three or more copies of the SMN2 gene will typically mean a less severe expression.

Antioxidants

In another embodiment, the antioxidant of the present invention is a vitamin. In another embodiment, the vitamin is a retinoid. In another embodiment, the retinoid is vitamin A. In another embodiment, vitamin A is a yellow fat-soluble, antioxidant vitamin. In another embodiment, vitamin A is obtained from animal sources. In another embodiment, vitamin A is obtained from milk. In another embodiment, vitamin A is obtained from eggs.

In another embodiment, vitamin A is pro-vitamin A-carotenoids. In another embodiment, vitamin A is obtained from plants. In another embodiment, pro-vitamin “A” carotenoids can be cleaved to produce retinal. In another embodiment, retinal is reversibly reduced to produce retinol or it can be irreversibly oxidized to produce retinoic acid. In some embodiments, active retinoid metabolites comprise 11-cis-retinal and the all-trans and 9-cis-isomers of retinoic acid.

In another embodiment, vitamin A comprises geometric isomers of retinol, retinal and retinoic acid. In another embodiment, vitamin A comprises isotretinoin. In another embodiment, vitamin A comprises all-trans retinoic acid (ATRA).

In another embodiment, amounts of vitamin A are measured in Retinal Equivalents (RE). In another embodiment, 1 RE is equivalent to 0.001 mg of retinal, or 0.006 mg of beta-carotene, or 3.3 International Units of vitamin A.

In another embodiment, vitamin A of the present invention is extracted from sweet potatoes. In another embodiment, vitamin A of the present invention is extracted from carrots. In another embodiment, vitamin A of the present invention is extracted from collard greens. In another embodiment, vitamin A of the present invention is extracted from kale. In another embodiment, vitamin A of the present invention is extracted from pumpkin. In another embodiment, vitamin A of the present invention is extracted from spinach. In another embodiment, vitamin A of the present invention is extracted from squash. In another embodiment, vitamin A of the present invention is extracted from apricots. In another embodiment, vitamin A of the present invention is extracted from cantaloupe melon. In another embodiment, vitamin A of the present invention is extracted from mango. In another embodiment, vitamin A of the present invention is extracted from broccoli. In another embodiment, vitamin A of the present invention is extracted from beef liver. In another embodiment, vitamin A of the present invention is extracted from pork liver. In another embodiment, vitamin A of the present invention is extracted from chicken liver. In another embodiment, vitamin A of the present invention is extracted from turkey liver. In another embodiment, vitamin A of the present invention is extracted chicken eggs. Each possibility represents a separate embodiment of the present invention.

In another embodiment vitamin A of the present invention is synthetic retinal. In another embodiment, the synthetic paths of retinal are known to a person of skill in the art. In another embodiment, Synthetic vitamin A concentrate consists of an ester or mixture of esters of retinol (the acetate, propionate or palmitate. In another embodiment, synthetic vitamin A concentrate is in an oily form. In another embodiment, synthetic vitamin A is diluted with a suitable vegetable oil. In another embodiment, synthetic vitamin A contains in 1 g not less than 500000 units of vitamin A and not less than 95.0% and not more than 110.0% of the number of units of vitamin A stated on the label. In another embodiment, synthetic vitamin A contains suitable stabilizing agents. In some embodiments, stabilizing agents comprise antioxidants. Each possibility represents a separate embodiment of the present invention.

In another embodiment, synthetic vitamin A concentrate (powder form) consists of an ester or mixture of esters of retinol (the acetate, propionate or palmitate) prepared by synthesis. In another embodiment, synthetic vitamin A is in a powder form. In another embodiment, synthetic vitamin A is dispersed in a matrix of gelatin. In another embodiment, synthetic vitamin A is dispersed in acacia. In another embodiment, synthetic vitamin A is dispersed in other suitable materials. In another embodiment, 1 g of synthetic vitamin A in a powder form contains not less than 250000 units of vitamin A and not less than 95.0% and not more than 115.0% of the number of units of vitamin A stated on the label. In another embodiment, powder form synthetic vitamin A contains suitable stabilizing agents. In some embodiments, stabilizing agents comprise antioxidants. Each possibility represents a separate embodiment of the present invention.

In some embodiments, the synthetic vitamin A concentrate is in a water-dispersible form. In some embodiments, vitamin A concentrate in a water-dispersible form comprises suitable solubilizers. In another embodiment, 1 g of vitamin A concentrate in a water-dispersible form contains not less than 100000 units of vitamin A and not less than 95.0% and not more than 115.0% of the number of units of vitamin A stated on the label. In another embodiment, water-dispersible form synthetic vitamin A contains suitable stabilizing agents. In some embodiments, stabilizing agents comprise antioxidants and antimicrobial preservatives.

In another embodiment, effective dose of vitamin A is measured in international units (IU). In another embodiment, 1 IU refers to biological activity and therefore is unique to each individual compound. In another embodiment, 1 IU of retinol is equivalent to approximately 0.3 micrograms (300 nanograms). In another embodiment, the upper limit dose of vitamin A is 4,000 μg/day. In another embodiment, the upper limit dose of vitamin A is 3,000 μg/day. In another embodiment, the upper limit dose of vitamin A is 2,300 μg/day.

In another embodiment, vitamin A dosage according to the present invention is 0.1-4000 μg/day. In another embodiment, vitamin A dosage according to the present invention is 100-4000 μg/day. In another embodiment, vitamin A dosage according to the present invention is 200-3000 μg/day. In another embodiment, vitamin A dosage according to the present invention is 400-2500 μg/day. In another embodiment, vitamin A dosage according to the present invention is 500-2000 μg/day. In another embodiment, vitamin A dosage according to the present invention is 700-1800 μg/day. In another embodiment, vitamin A dosage according to the present invention is 700-1300 μg/day. Each possibility represents a separate embodiment of the present invention.

In another embodiment, the vitamin of the present invention is vitamin C or the L-enantiomer of ascorbate. In some embodiments, vitamin C of the present invention derives from meat. In another embodiment, vitamin C of the present invention derives from liver. In some embodiments, vitamin C of the present invention derives from fruits or vegetables. In another embodiment, vitamin C of the present invention derives from camu camu fruit. In another embodiment, vitamin C of the present invention derives from billygoat plum. In another embodiment, vitamin C of the present invention derives from Wolfberry. In another embodiment, vitamin C of the present invention derives from Rose hip. In another embodiment, vitamin C of the present invention derives from acerola. In another embodiment, vitamin C of the present invention derives from amla. In another embodiment, vitamin C of the present invention derives from jujube. In another embodiment, vitamin C of the present invention derives from baobab. In another embodiment, vitamin C of the present invention derives from blackcurrant. In another embodiment, vitamin C of the present invention derives from red pepper. In another embodiment, vitamin C of the present invention derives from parsley. In another embodiment, vitamin C of the present invention derives from seabuckthorn. In another embodiment, vitamin C of the present invention derives from guava. In another embodiment, vitamin C of the present invention derives from kiwi. In another embodiment, vitamin C of the present invention derives from broccoli. In another embodiment, vitamin C of the present invention derives from loganberry. Each possibility represents a separate embodiment of the present invention.

In another embodiment, vitamin C as ascorbic acid is in the form of crystals. In another embodiment, vitamin C is in the form of various mineral ascorbates.

In another embodiment, vitamin C of the present invention is produced from glucose by two main routes. In another embodiment, the Reichstein process is used. In some embodiments, a two-step fermentation process is used. In another embodiment, the processes at least 40% vitamin C from the glucose feed. In another embodiment, the processes at least 50% vitamin C from the glucose feed. In another embodiment, the processes at least 60% vitamin C from the glucose feed. In another embodiment, the processes at least 70% vitamin C from the glucose feed. Each possibility represents a separate embodiment of the present invention.

In another embodiment, vitamin C of the present invention is administered at a dosage of 20-200000 mg/day. In another embodiment, vitamin C of the present invention is administered at a dosage of 20-100000 mg/day. In another embodiment, vitamin C of the present invention is administered at a dosage of 20-50000 mg/day. In another embodiment, vitamin C of the present invention is administered at a dosage of 20-50000 mg/day. In another embodiment, vitamin C of the present invention is administered at a dosage of 20-25000 mg/day. In another embodiment, vitamin C of the present invention is administered at a dosage of 20-20000 mg/day. In another embodiment, vitamin C of the present invention is administered at a dosage of 40-20000 mg/day. In another embodiment, vitamin C of the present invention is administered at a dosage of 50-20000 mg/day. In another embodiment, vitamin C of the present invention is administered at a dosage of 50-10000 mg/day. In another embodiment, vitamin C of the present invention is administered at a dosage of 100-10000 mg/day. In another embodiment, vitamin C of the present invention is administered at a dosage of 100-5000 mg/day. In another embodiment, vitamin C of the present invention is administered at a dosage of 100-4000 mg/day. In another embodiment, vitamin C of the present invention is administered at a dosage of 100-3000 mg/day. In another embodiment, the highest dose for a given subject (thousands of milligrams) may result in diarrhea which indicates the body's true vitamin C requirement. Each possibility represents a separate embodiment of the present invention.

In another embodiment, the vitamin of the present invention is vitamin E. In another embodiment, vitamin E of the present invention is obtained from vegetable oils. In another embodiment, vitamin E of the present invention is obtained from palm oil. In another embodiment, vitamin E of the present invention is obtained from sunflower oil. In another embodiment, vitamin E of the present invention is obtained from soybean oil. In another embodiment, vitamin E of the present invention is obtained from corn oil. In another embodiment, vitamin E of the present invention is obtained from olive oil. In another embodiment, vitamin E of the present invention is obtained from nuts. In another embodiment, vitamin E of the present invention is obtained from sunflower seeds. In another embodiment, vitamin E of the present invention is obtained from kiwi. In another embodiment, vitamin E of the present invention is obtained from wheat germ. In another embodiment, vitamin E of the present invention is obtained from seabuckthorn berries. In another embodiment, vitamin E of the present invention is obtained from fish. In another embodiment, vitamin E of the present invention is obtained from green leafy vegetables. Each possibility represents a separate embodiment of the present invention.

In another embodiment, vitamin E of the present invention is alpha-tocopherol. In another embodiment, vitamin E is measured in international units (IU). In another embodiment, 1 IU of vitamin E is defined as the biological equivalent of 0.667 milligrams of RRR-alpha-tocopherol (formerly named d-alpha-tocopherol, or of 1 milligram of all-rac-alpha-tocopheryl acetate commercially called dl-alpha-tocopheryl acetate.

In another embodiment, vitamin E of the present invention is a tocotrienol. In another embodiment, tocotrienols have structures corresponding to the four tocopherols, except with an unsaturated bond in each of the three isoprene units that form the hydrocarbon tail.

In another embodiment, fully synthetic vitamin E, is “d, 1-alpha-tocopherol”. In another embodiment, d, 1-alpha-tocopherol is the acetate ester. In another embodiment, semi-synthetic vitamin E esters, is highly fractionated natural d-alpha tocopherol, less fractionated “natural mixed tocopherols” and high gamma-tocopherol fraction supplements. In another embodiment, synthetic vitamin E of the present invention is manufactured as all-racemic alpha tocopheryl acetate with three chiral centers, with only one alpha tocopherol molecule (moiety) in 8 molecules as actual R, R,R-alpha tocopherol. In another embodiment, synthetic all-rac vitamin E is 50% d-alpha tocopherol moiety and 50% 1-alpha-tocopherol moiety, as synthesized by an earlier process with only one chiral center.

In another embodiment, semi-synthetic vitamin E is synthesized by converting the co μM on natural beta, gamma and delta tocopherol isomers into esters using acetic acid. In another embodiment, semi-synthetic vitamin E is synthesized by converting the co μM on natural beta, gamma and delta tocopherol isomers into esters using succinic acid. In some embodiments, methyl groups are added to yield d-alpha tocopheryl esters such as d-alpha tocopheryl acetate or d-alpha tocopheryl succinate.

In another embodiment, vitamin E of the present invention is in the form of mixed tocopherols. In another embodiment, mixed tocopherols comprise at least 10% w/w other natural R, R,R-tocopherols, i.e. R, R,R-alpha-tocopherol content plus at least 15% R, R,R-beta-, R, R,R-gamma-, R, R,R-delta-tocopherols. In another embodiment, mixed tocopherols comprise at least 15% w/w other natural R, R,R-tocopherols, i.e. R, R,R-alpha-tocopherol content plus at least 20% R, R,R-beta-, R, R,R-gamma-, R, R,R-delta-tocopherols. In another embodiment, mixed tocopherols comprise at least 20% w/w other natural R, R,R-tocopherols, i.e. R, R,R-alpha-tocopherol content plus at least 25% R, R,R-beta-, R, R,R-gamma-, R, R,R-delta-tocopherols. In another embodiment, mixed tocopherols comprise at least 25% w/w other natural R, R,R-tocopherols, i.e. R, R,R-alpha-tocopherol content plus at least 30% R, R,R-beta-, R, R,R-gamma-, R, R,R-delta-tocopherols. In another embodiment, mixed tocopherols comprise at least 30% w/w other natural R, R,R-tocopherols, i.e. R, R,R-alpha-tocopherol content plus at least 35% R, R,R-beta-, R, R,R-gamma-, R, R,R-delta-tocopherols. In another embodiment, mixed tocopherols comprise at least 40% w/w other natural R, R,R-tocopherols, i.e. R, R,R-alpha-tocopherol content plus at least 40% R, R,R-beta-, R, R,R-gamma-, R, R,R-delta-tocopherols. In another embodiment, mixed tocopherols comprise at least 50% w/w other natural R, R,R-tocopherols, i.e. R, R,R-alpha-tocopherol content plus at least 50% R, R,R-beta-, R, R,R-gamma-, R, R,R-delta-tocopherols. In another embodiment, mixed tocopherols comprise 200% w/w or more of the other tocopherols and measurable tocotrienols. In another embodiment, mixed tocopherols comprise higher gamma-tocopherol content. Each possibility represents a separate embodiment of the present invention.

In another embodiment, vitamin E of the present invention is administered at a dosage 1-150 mg/day. In another embodiment, vitamin E of the present invention is administered at a dosage of 5-150 mg/day based on the alpha-tocopherol form. In another embodiment, vitamin E of the present invention is administered at a dosage of 7-100 mg/day based on the alpha-tocopherol form. In another embodiment, vitamin E of the present invention is administered at a dosage of 10-80 mg/day based on the alpha-tocopherol form. In another embodiment, vitamin E of the present invention is administered at a dosage of 20-60 mg/day based on the alpha-tocopherol form. Each possibility represents a separate embodiment of the present invention.

In another embodiment, the antioxidant of the present invention is Coenzyme Q10 (also known as ubiquinone, ubidecarenone, vitamin Q10 or, CoQ10). In another embodiment, coenzyme Q10 is synthesized in yeast strains.

In another embodiment, Coenzyme Q10 of the present invention is administered at a dosage of 10-400 mg/day. In another embodiment, Coenzyme Q10 of the present invention is administered at a dosage of 10-300 mg/day. In another embodiment, Coenzyme Q10 of the present invention is administered at a dosage of 20-300 mg/day. In another embodiment, Coenzyme Q10 of the present invention is administered at a dosage of 30-300 mg/day. In another embodiment, Coenzyme Q10 of the present invention is administered at a dosage of 50-300 mg/day. In another embodiment, Coenzyme Q10 of the present invention is administered at a dosage of 100-400 mg/day. In another embodiment, Coenzyme Q10 of the present invention is administered at a dosage of 100-300 mg/day. In another embodiment, Coenzyme Q10 of the present invention is administered at a dosage of 150-300 mg/day. Each possibility represents a separate embodiment of the present invention.

In another embodiment, the antioxidant of the present invention is manganese. In another embodiment, manganese of the present invention is obtained from dietary sources. In another embodiment, the dietary source is nuts. In another embodiment, the dietary source is seeds. In another embodiment, the dietary source is wheat germ. In another embodiment, the dietary source is whole grains. In another embodiment, the dietary source is legumes. In another embodiment, the dietary source is pineapples. Each possibility represents a separate embodiment of the present invention.

In another embodiment, manganese of the present invention is a manganese salt. In another embodiment, manganese salt of the present invention is sulfate. In another embodiment, manganese salt of the present invention is gluconate.

In another embodiment, manganese of the present invention is manganese chelate. In another embodiment, manganese chelate of the present invention is aspartate. In another embodiment, manganese chelate of the present invention is picolinate. In another embodiment, manganese chelate of the present invention is fumarate. In another embodiment, manganese chelate of the present invention is malate. In another embodiment, manganese chelate of the present invention is succinate. In another embodiment, manganese chelate of the present invention is citrate. In another embodiment, manganese chelate of the present invention is an amino acid chelate. Each possibility represents a separate embodiment of the present invention.

In some embodiments, manganese of the present invention is formulated as tablets. In some embodiments, manganese of the present invention is formulated as capsules. In some embodiments, manganese dosage of the present invention ranges from 0.1-25 mg/day. In another embodiment, manganese dosage of the present invention ranges from 0.1-20 mg/day. In another embodiment, manganese dosage of the present invention ranges from 0.3-20 mg/day. In another embodiment, manganese dosage of the present invention ranges from 0.6-25 mg/day. In another embodiment, manganese dosage of the present invention ranges from 1.2-25 mg/day. In another embodiment, manganese dosage of the present invention ranges from 1.5-15 mg/day. In another embodiment, manganese dosage of the present invention ranges from 1.5-12 mg/day. In another embodiment, manganese dosage of the present invention ranges from 1.5-10 mg/day. In another embodiment, manganese dosage of the present invention ranges from 1.5-6 mg/day. Each possibility represents a separate embodiment of the present invention.

In another embodiment, the antioxidant of the present invention is a hormone. In another embodiment, the hormone is melatonin. In some embodiments, melatonin of the present invention is formulated as tablets. In some embodiments, melatonin of the present invention is formulated as capsules. In another embodiment, melatonin of the present invention is formulated as a quick-release tablet. In another embodiment, melatonin of the present invention is formulated as a quick-release capsule. In another embodiment, melatonin of the present invention is formulated as a sustained-release tablet. In another embodiment, melatonin of the present invention is formulated as a sustained-release capsule. In another embodiment, melatonin if formulated as an injectable. In another embodiment, melatonin of the present invention is injected intramuscularly. In another embodiment, melatonin of the present invention is formulated as a solution. In another embodiment, melatonin of the present invention is formulated as an intranasal solution. Each possibility represents a separate embodiment of the present invention.

In some embodiments, melatonin of the present invention is natural melatonin. In another embodiment, natural melatonin is derived from actual extracts of the pineal gland of an animal. In another embodiment, the actual extracts of the pineal gland are derived from bovine. In another embodiment, synthetic melatonin of the present invention is produced from pharmaceutical grade ingredients. Each possibility represents a separate embodiment of the present invention.

In some embodiments, melatonin dosage of the present invention ranges from 0.1-50 mg/day. In another embodiment, melatonin dosage of the present invention ranges from 0.1-40 mg/day. In another embodiment, melatonin dosage of the present invention ranges from 0.3-40 mg/day. In another embodiment, melatonin dosage of the present invention ranges from 0.5-40 mg/day. In another embodiment, melatonin dosage of the present invention ranges from 0.5-35 mg/day. In another embodiment, melatonin dosage of the present invention ranges from 1-35 mg/day. In another embodiment, melatonin dosage of the present invention ranges from 1.5-35 mg/day. In another embodiment, melatonin dosage of the present invention ranges from 2-30 mg/day. In another embodiment, melatonin dosage of the present invention ranges from 2.5-30 mg/day. Each possibility represents a separate embodiment of the present invention.

In some embodiments, the antioxidant of the present invention is a carotenoid. In another embodiment, the carotenoid is beta-carotene. In another embodiment, beta-carotene of the present invention is formulated in an oil matrix gelatin capsule. In another embodiment, beta-carotene of the present invention is formulated in a water-miscible form. In another embodiment, water-miscible forms comprise water-miscible beta-carotene beadlets. In another embodiment, beta-carotene of the present invention is formulated as tablets. In another embodiment, beta-carotene of the present invention is formulated as chewable tablets. Each possibility represents a separate embodiment of the present invention.

In some embodiments, beta-carotene of the present invention is obtained from fruit and vegetables. In some embodiments, beta-carotene of the present invention is synthetic beta-carotene. In some embodiments, beta-carotene of the present invention is synthetic all-trans beta-carotene. In some embodiments, beta-carotene of the present invention is beta- and alpha-carotene from the algae. In some embodiments, beta-carotene of the present invention is beta- and alpha-carotene from Dunaliella. In some embodiments, beta-carotene of the present invention is mixed carotenes from palm oil. Each possibility represents a separate embodiment of the present invention.

In some embodiments, beta-carotene dosage of the present invention ranges from 5-350 mg/day. In another embodiment, beta-carotene dosage of the present invention ranges from 10-350 mg/day. In another embodiment, beta-carotene dosage of the present invention ranges from 10-300 mg/day. In another embodiment, beta-carotene dosage of the present invention ranges from 15-300 mg/day. In another embodiment, beta-carotene dosage of the present invention ranges from 15-280 mg/day. In another embodiment, beta-carotene dosage of the present invention ranges from 20-280 mg/day. In another embodiment, beta-carotene dosage of the present invention ranges from 20-270 mg/day. In another embodiment, beta-carotene dosage of the present invention ranges from 30-250 mg/day. In another embodiment, beta-carotene dosage of the present invention ranges from 40-200 mg/day. Each possibility represents a separate embodiment of the present invention.

In some embodiments, the antioxidant of the present invention is a carotenoid. In another embodiment, the carotenoid of the present invention is alpha-carotene. In some embodiments, alpha-carotene. In another embodiment, alpha-carotene of the present invention is formulated in a capsule. In another embodiment, alpha-carotene of the present invention is formulated as tablets. In another embodiment, alpha-carotene of the present invention is formulated as chewable tablets. Each possibility represents a separate embodiment of the present invention.

In some embodiments, alpha-carotene of the present invention is obtained from orange- and red-colored fruits and vegetables. In another embodiment, alpha-carotene of the present invention is obtained from carrots. In another embodiment, alpha-carotene of the present invention is obtained from sweet potatoes. In another embodiment, alpha-carotene of the present invention is obtained from squash. In another embodiment, alpha-carotene of the present invention is obtained from broccoli. In another embodiment, alpha-carotene of the present invention is obtained from kale. In another embodiment, alpha-carotene of the present invention is obtained from cantaloupe. In another embodiment, alpha-carotene of the present invention is obtained from Brussels sprouts. In another embodiment, alpha-carotene of the present invention is obtained from kiwi. In another embodiment, alpha-carotene of the present invention is obtained from spinach. In another embodiment, alpha-carotene of the present invention is obtained from mangos. Each possibility represents a separate embodiment of the present invention.

In some embodiments, lycopene dosage of the present invention ranges from 1-500 mg/day. In another embodiment, lycopene dosage of the present invention ranges from 1-400 mg/day. In another embodiment, lycopene dosage of the present invention ranges from 5-400 mg/day. In another embodiment, lycopene dosage of the present invention ranges from 5-350 mg/day. In another embodiment, lycopene dosage of the present invention ranges from 10-300 mg/day. In another embodiment, lycopene dosage of the present invention ranges from 10-280 mg/day. In another embodiment, lycopene dosage of the present invention ranges from 10-250 mg/day. In another embodiment, lycopene dosage of the present invention ranges from 20-200 mg/day. Each possibility represents a separate embodiment of the present invention.

In some embodiments, the antioxidant of the present invention is a carotenoid. In another embodiment, the carotenoid of the present invention is lycopene. In some embodiments, lycopene of the present invention is in an oleoresin preparation. In some embodiments, lycopene of the present invention is in a phospholipid preparation. In some embodiments, lycopene of the present invention is in an oil preparation such as medium chain triglycerides. In another embodiment, lycopene of the present invention is formulated in a gelatin capsule. In another embodiment, lycopene of the present invention is formulated as tablets. In another embodiment, lycopene of the present invention is formulated as chewable tablets. Each possibility represents a separate embodiment of the present invention.

In some embodiments, lycopene of the present invention is obtained from fruit and vegetables. In some embodiments, lycopene of the present invention is obtained from tomatoes. In another embodiment, lycopene of the present invention is obtained from pink guava. In another embodiment, lycopene of the present invention is obtained from watermelon. In another embodiment, lycopene of the present invention is obtained from pink grapefruit. In another embodiment, lycopene of the present invention is obtained from papaya. Each possibility represents a separate embodiment of the present invention.

In some embodiments, lycopene dosage of the present invention ranges from 1-150 mg/day. In another embodiment, lycopene dosage of the present invention ranges from 1-120 mg/day. In another embodiment, lycopene dosage of the present invention ranges from 1-100 mg/day. In another embodiment, lycopene dosage of the present invention ranges from 5-100 mg/day. In another embodiment, lycopene dosage of the present invention ranges from 5-80 mg/day. In another embodiment, lycopene dosage of the present invention ranges from 10-70 mg/day. In another embodiment, lycopene dosage of the present invention ranges from 10-50 mg/day. Each possibility represents a separate embodiment of the present invention.

In some embodiments, the antioxidant of the present invention is lutein. In some embodiments, lutein of the present invention is lutein diester. In another embodiment, lutein of the present invention is unesterified lutein. In some embodiments, lutein of the present invention is purified crystalline lutein. In another embodiment, lutein of the present invention is formulated in a gelatin capsule. In another embodiment, lutein of the present invention is formulated as tablets. In another embodiment, lutein of the present invention is formulated as chewable tablets. Each possibility represents a separate embodiment of the present invention.

In some embodiments, lutein of the present invention is obtained from kale. In another embodiment, lutein of the present invention is obtained from spinach. In another embodiment, lutein of the present invention is obtained from collards. In another embodiment, lutein of the present invention is obtained from turnip greens. In another embodiment, lutein of the present invention is obtained from lettuce. Each possibility represents a separate embodiment of the present invention.

In some embodiments, lutein dosage of the present invention ranges from 0.1-90 mg/day. In another embodiment, lutein dosage of the present invention ranges from 0.2-70 mg/day. In another embodiment, lutein dosage of the present invention ranges from 0.5-50 mg/day. In another embodiment, lutein dosage of the present invention ranges from 1-50 mg/day. In another embodiment, lutein dosage of the present invention ranges from 2-50 mg/day. In another embodiment, lutein dosage of the present invention ranges from 5-40 mg/day. In another embodiment, lutein dosage of the present invention ranges from 5-30 mg/day. Each possibility represents a separate embodiment of the present invention.

In some embodiments, the antioxidant of the present invention is zeaxanthin. In another embodiment, zeaxanthin of the present invention is formulated in a gelatin capsule. In another embodiment, zeaxanthin of the present invention is formulated as tablets. In another embodiment, zeaxanthin of the present invention is formulated as chewable tablets. Each possibility represents a separate embodiment of the present invention.

In some embodiments, zeaxanthin of the present invention is obtained from corn. In another embodiment, zeaxanthin of the present invention is obtained from spinach. In another embodiment, zeaxanthin of the present invention is obtained from collards. In another embodiment, zeaxanthin of the present invention is obtained from oranges. In another embodiment, zeaxanthin of the present invention is obtained from lettuce. Each possibility represents a separate embodiment of the present invention.

In some embodiments, zeaxanthin dosage of the present invention ranges from 0.1-250 mg/day. In another embodiment, zeaxanthin dosage of the present invention ranges from 0.5-200 mg/day. In another embodiment, zeaxanthin dosage of the present invention ranges from 1-150 mg/day. In another embodiment, zeaxanthin dosage of the present invention ranges from 1-100 mg/day. In another embodiment, zeaxanthin dosage of the present invention ranges from 2-100 mg/day. In another embodiment, zeaxanthin dosage of the present invention ranges from 5-80 mg/day. In another embodiment, zeaxanthin dosage of the present invention ranges from 15-80 mg/day. Each possibility represents a separate embodiment of the present invention.

In some embodiments, the antioxidant of the present invention is astaxanthin. In another embodiment, astaxanthin of the present invention is formulated in a gelatin capsule. In another embodiment, astaxanthin of the present invention is formulated as tablets. In another embodiment, astaxanthin of the present invention is formulated as chewable tablets. Each possibility represents a separate embodiment of the present invention.

In some embodiments, astaxanthin of the present invention is obtained from microscopic small plants. In another embodiment, the plant is the micro-alga Haematococcus pluvialis. In some embodiments, astaxanthin is synthetic astaxanthin. Each possibility represents a separate embodiment of the present invention.

In some embodiments, astaxanthin dosage of the present invention ranges from 0.1-100 mg/day. In another embodiment, astaxanthin dosage of the present invention ranges from 0.5-100 mg/day. In another embodiment, astaxanthin dosage of the present invention ranges from 1-80 mg/day. In another embodiment, astaxanthin dosage of the present invention ranges from 2-50 mg/day. In another embodiment, astaxanthin dosage of the present invention ranges from 2-40 mg/day. In another embodiment, astaxanthin dosage of the present invention ranges from 10-60 mg/day. In another embodiment, astaxanthin dosage of the present invention ranges from 15-30 mg/day. Each possibility represents a separate embodiment of the present invention.

In some embodiments, the antioxidant of the present invention is canthaxanthin. In another embodiment, canthaxanthin of the present invention is formulated in a gelatin capsule. In another embodiment, canthaxanthin of the present invention is formulated as tablets. In another embodiment, canthaxanthin of the present invention is formulated as chewable tablets. Each possibility represents a separate embodiment of the present invention.

In some embodiments, canthaxanthin of the present invention is obtained from mushrooms. In another embodiment, canthaxanthin is synthetic canthaxanthin. Each possibility represents a separate embodiment of the present invention.

In some embodiments, canthaxanthin dosage of the present invention ranges from 0.1-200 mg/day. In another embodiment, canthaxanthin dosage of the present invention ranges from 1-1180 mg/day. In another embodiment, canthaxanthin dosage of the present invention ranges from 1-150 mg/day. In another embodiment, canthaxanthin dosage of the present invention ranges from 1-100 mg/day. In another embodiment, canthaxanthin dosage of the present invention ranges from 1-50 mg/day. In another embodiment, canthaxanthin dosage of the present invention ranges from 2-50 mg/day. In another embodiment, canthaxanthin dosage of the present invention ranges from 5-50 mg/day. In another embodiment, canthaxanthin dosage of the present invention ranges from 5-40 mg/day. In another embodiment, canthaxanthin dosage of the present invention ranges from 5-30 mg/day. Each possibility represents a separate embodiment of the present invention.

In some embodiments, the antioxidant of the present invention is a flavone. In another embodiment, the flavone of the present invention is luteolin. In another embodiment, luteolin of the present invention is formulated in a gelatin capsule. In another embodiment, luteolin of the present invention is formulated as tablets. In another embodiment, luteolin of the present invention is formulated as chewable tablets. Each possibility represents a separate embodiment of the present invention.

In another embodiment, luteolin of the present invention is obtained from olive oil. In another embodiment, luteolin of the present invention is obtained from green pepper. In another embodiment, luteolin of the present invention is obtained from perilla plant. In another embodiment, luteolin of the present invention is synthetic luteolin. In another embodiment, luteolin of the present invention is semi-synthetic luteolin. Each possibility represents a separate embodiment of the present invention.

In some embodiments, luteolin dosage of the present invention ranges from 0.1-250 mg/day. In another embodiment, luteolin dosage of the present invention ranges from 0.5-150 mg/day. In another embodiment, luteolin dosage of the present invention ranges from 1-120 mg/day. In another embodiment, luteolin dosage of the present invention ranges from 2-80 mg/day. In another embodiment, luteolin dosage of the present invention ranges from 2-60 mg/day. In another embodiment, luteolin dosage of the present invention ranges from 2-50 mg/day. In another embodiment, luteolin dosage of the present invention ranges from 4-60 mg/day. In another embodiment, luteolin dosage of the present invention ranges from 5-40 mg/day. Each possibility represents a separate embodiment of the present invention.

In another embodiment, the flavone of the present invention is apigenin. In another embodiment, apigenin of the present invention is formulated in a gelatin capsule. In another embodiment, apigenin of the present invention is formulated as tablets. In another embodiment, apigenin of the present invention is formulated as chewable tablets. Each possibility represents a separate embodiment of the present invention.

In another embodiment, apigenin of the present invention is obtained leaves, seeds and fruits of flowering plants. In another embodiment, apigenin of the present invention are obtained from tea leaves. Each possibility represents a separate embodiment of the present invention.

In some embodiments, apigenin dosage of the present invention ranges from 0.1-150 mg/day. In another embodiment, apigenin dosage of the present invention ranges from 0.5-150 mg/day. In another embodiment, apigenin dosage of the present invention ranges from 1-120 mg/day. In another embodiment, apigenin dosage of the present invention ranges from 2-100 mg/day. In another embodiment, apigenin dosage of the present invention ranges from 2-80 mg/day. In another embodiment, apigenin dosage of the present invention ranges from 2-60 mg/day. In another embodiment, apigenin dosage of the present invention ranges from 2-50 mg/day. In another embodiment, apigenin dosage of the present invention ranges from 4-60 mg/day. Each possibility represents a separate embodiment of the present invention.

In another embodiment, the flavone of the present invention is tangeritin. In another embodiment, tangeritin of the present invention is formulated in a gelatin capsule. In another embodiment, tangeritin of the present invention is formulated as tablets. In another embodiment, tangeritin of the present invention is formulated as chewable tablets. Each possibility represents a separate embodiment of the present invention.

In another embodiment, tangeritin of the present invention is obtained from citrus fruits. In another embodiment, tangeritin of the present invention is synthetic tangeritin.

In some embodiments, tangeritin dosage of the present invention ranges from 0.1-150 mg/day. In another embodiment, tangeritin dosage of the present invention ranges from 0.5-150 mg/day. In another embodiment, tangeritin dosage of the present invention ranges from 1-120 mg/day. In another embodiment, tangeritin dosage of the present invention ranges from 2-100 mg/day. In another embodiment, tangeritin dosage of the present invention ranges from 2-80 mg/day. In another embodiment, tangeritin dosage of the present invention ranges from 4-80 mg/day. In another embodiment, tangeritin dosage of the present invention ranges from 2-60 mg/day. In another embodiment, tangeritin dosage of the present invention ranges from 4-50 mg/day. In another embodiment, tangeritin dosage of the present invention ranges from 6-100 mg/day. Each possibility represents a separate embodiment of the present invention.

In some embodiments, the antioxidant of the present invention is a flavonol. In another embodiment, the flavonol of the present invention is quercetin. In another embodiment, the flavonol of the present invention is kaempferol. In another embodiment, the flavonol of the present invention is myricetin. In another embodiment, the flavonol of the present invention is fiestin. In another embodiment, the flavonol of the present invention is a flavanols polymer. Each possibility represents a separate embodiment of the present invention.

In another embodiment, a flavonol of the present invention is formulated in a gelatin capsule. In another embodiment, a flavonol of the present invention is formulated as tablets. In another embodiment, a flavonol of the present invention is formulated as chewable tablets.

In another embodiment, a flavonol of the present invention is obtained from walnuts. In another embodiment, a flavonol of the present invention is obtained from onions. In another embodiment, a flavonol of the present invention is obtained Lollo Rosso lettuce. In another embodiment, a flavonol of the present invention is synthetic flavonol. Each possibility represents a separate embodiment of the present invention.

In some embodiments, flavonol dosage of the present invention ranges from 0.1-150 mg/day. In another embodiment, flavonol dosage of the present invention ranges from 0.5-150 mg/day. In another embodiment, flavonol dosage of the present invention ranges from 1-125 mg/day. In another embodiment, flavonol dosage of the present invention ranges from 2-100 mg/day. In another embodiment, flavonol dosage of the present invention ranges from 2-80 mg/day. In another embodiment, flavonol dosage of the present invention ranges from 4-80 mg/day. In another embodiment, flavonol dosage of the present invention ranges from 5-100 mg/day. In another embodiment, flavonol dosage of the present invention ranges from 10-80 mg/day. Each possibility represents a separate embodiment of the present invention.

In some embodiments, the antioxidant of the present invention is a flavanone. In another embodiment, the flavanone of the present invention is hesperetin. In another embodiment, the flavanone of the present invention is naringenin. In another embodiment, the flavanone of the present invention is eriodictyol. In another embodiment, a flavanone of the present invention is formulated in a gelatin capsule. In another embodiment, a flavanone of the present invention is formulated as tablets. In another embodiment, a flavanone of the present invention is formulated as chewable tablets. Each possibility represents a separate embodiment of the present invention.

In another embodiment, a flavanone of the present invention is obtained from citrus fruits. In another embodiment, a flavanone of the present invention is obtained from oranges. In another embodiment, a flavanone of the present invention is obtained from grapefruits. In another embodiment, a flavanone of the present invention is obtained from lemons. In another embodiment, a flavanone of the present invention is synthetic flavanone. Each possibility represents a separate embodiment of the present invention.

In some embodiments, flavanone dosage of the present invention ranges from 5-1500 mg/day. In another embodiment, flavanone dosage of the present invention ranges from 10-1500 mg/day. In another embodiment, flavanone dosage of the present invention ranges from 10-1200 mg/day. In another embodiment, flavanone dosage of the present invention ranges from 50-1000 mg/day. In another embodiment, flavanone dosage of the present invention ranges from 70-1000 mg/day. In another embodiment, flavanone dosage of the present invention ranges from 70-800 mg/day. In another embodiment, flavanone dosage of the present invention ranges from 70-700 mg/day. Each possibility represents a separate embodiment of the present invention.

In some embodiments, the antioxidant of the present invention is an isoflavone. In another embodiment, the isoflavone of the present invention is daidzein. In another embodiment, the isoflavone of the present invention is, genistein. In another embodiment, the isoflavone of the present invention is glycitein. In another embodiment, an isoflavone of the present invention is formulated in a gelatin capsule. In another embodiment, an isoflavone of the present invention is formulated as tablets. In another embodiment, an isoflavone of the present invention is formulated as chewable tablets. Each possibility represents a separate embodiment of the present invention.

In another embodiment, an isoflavone of the present invention is obtained from a soy source. In another embodiment, an isoflavone of the present invention is obtained from soy cheese. In another embodiment, an isoflavone of the present invention is obtained from soy flower. In another embodiment, an isoflavone of the present invention is obtained from tofu. In another embodiment, an isoflavone of the present invention is synthetic isoflavone. Each possibility represents a separate embodiment of the present invention.

In some embodiments, isoflavone dosage of the present invention ranges from 5-1800 mg/day. In another embodiment, isoflavone dosage of the present invention ranges from 10-1600 mg/day. In another embodiment, isoflavone dosage of the present invention ranges from 10-1200 mg/day. In another embodiment, isoflavone dosage of the present invention ranges from 50-1000 mg/day. In another embodiment, isoflavone dosage of the present invention ranges from 70-1000 mg/day. In another embodiment, isoflavone dosage of the present invention ranges from 70-800 mg/day. In another embodiment, isoflavone dosage of the present invention ranges from 80-800 mg/day. In another embodiment, isoflavone dosage of the present invention ranges from 100-1000 mg/day. Each possibility represents a separate embodiment of the present invention.

In some embodiments, the antioxidant of the present invention is a stilbenoid. In another embodiment, the stilbenoid of the present invention is reservatrol. In another embodiment, a stilbenoid of the present invention is formulated in a gelatin capsule. In another embodiment, a stilbenoid of the present invention is formulated as tablets. In another embodiment, a stilbenoid of the present invention is formulated as chewable tablets. Each possibility represents a separate embodiment of the present invention.

In another embodiment, a stilbenoid of the present invention is obtained from grapes. In another embodiment, a stilbenoid of the present invention is synthetic stilbenoid.

In some embodiments, stilbenoid dosage of the present invention ranges from 1-3000 mg/day. In another embodiment, stilbenoid dosage of the present invention ranges from 10-3000 mg/day. In another embodiment, stilbenoid dosage of the present invention ranges from 10-2500 mg/day. In another embodiment, stilbenoid dosage of the present invention ranges from 50-2000 mg/day. In another embodiment, stilbenoid dosage of the present invention ranges from 50-1500 mg/day. In another embodiment, stilbenoid dosage of the present invention ranges from 80-1500 mg/day. In another embodiment, stilbenoid dosage of the present invention ranges from 100-1500 mg/day. In another embodiment, stilbenoid dosage of the present invention ranges from 150-1500 mg/day. In another embodiment, stilbenoid dosage of the present invention ranges from 150-1000 mg/day. In another embodiment, stilbenoid dosage of the present invention ranges from 150-600 mg/day. Each possibility represents a separate embodiment of the present invention.

In another embodiment, the antioxidant of the present invention is a polyphenol antioxidant. In another embodiment, the polyphenol antioxidant is resveratrol. In another embodiment, the polyphenol antioxidant is ellagic acid. In another embodiment, the polyphenol antioxidant is gallic acid. In another embodiment, the polyphenol antioxidant is salicylic acid. In another embodiment, the polyphenol antioxidant is rosmarinic acid. In another embodiment, the polyphenol antioxidant is cinnamic acid. In another embodiment, the polyphenol antioxidant is chlorogenic acid. In another embodiment, the polyphenol antioxidant is chicoric acid. In another embodiment, the polyphenol antioxidant is gallotannin. In another embodiment, the polyphenol antioxidant is ellagitannin. In another embodiment, the polyphenol antioxidant is emblicanin. Each possibility represents a separate embodiment of the present invention.

In another embodiment, a polyphenol antioxidant of the present invention is formulated in a gelatin capsule. In another embodiment, a polyphenol antioxidant of the present invention is formulated as tablets. In another embodiment, a polyphenol antioxidant of the present invention is formulated as chewable tablets. Each possibility represents a separate embodiment of the present invention.

In another embodiment, a polyphenol antioxidant of the present invention is obtained from blackberries. In another embodiment, a polyphenol antioxidant of the present invention is obtained from legumes. In another embodiment, a polyphenol antioxidant of the present invention is obtained from apples. In another embodiment, a polyphenol antioxidant of the present invention is obtained from cantaloupe. In another embodiment, a polyphenol antioxidant of the present invention is obtained from pears. In another embodiment, a polyphenol antioxidant of the present invention is obtained from cherries. In another embodiment, a polyphenol antioxidant of the present invention is obtained from cranberries. In another embodiment, a polyphenol antioxidant of the present invention is obtained from grapes. In another embodiment, a polyphenol antioxidant of the present invention is obtained from plums. In another embodiment, a polyphenol antioxidant of the present invention is obtained from raspberries. In another embodiment, a polyphenol antioxidant of the present invention is obtained from strawberries. In another embodiment, a polyphenol antioxidant of the present invention is obtained from broccoli. In another embodiment, a polyphenol antioxidant of the present invention is obtained from cabbage. In another embodiment, a polyphenol antioxidant of the present invention is obtained from celery. In another embodiment, a polyphenol antioxidant of the present invention is obtained from onions. In another embodiment, a polyphenol antioxidant of the present invention is obtained from olive oil. In another embodiment, a polyphenol antioxidant of the present invention is obtained from chocolate. In another embodiment, a polyphenol antioxidant of the present invention is obtained from grains. In another embodiment, a polyphenol antioxidant of the present invention is synthetic polyphenol antioxidant. Each possibility represents a separate embodiment of the present invention.

In some embodiments, polyphenol antioxidant dosage of the present invention ranges from 1-3000 mg/day. In another embodiment, polyphenol antioxidant dosage of the present invention ranges from 10-3000 mg/day. In another embodiment, polyphenol antioxidant dosage of the present invention ranges from 10-2500 mg/day. In another embodiment, polyphenol antioxidant dosage of the present invention ranges from 50-2000 mg/day. In another embodiment, polyphenol antioxidant dosage of the present invention ranges from 50-1500 mg/day. In another embodiment, polyphenol antioxidant dosage of the present invention ranges from 80-1200 mg/day. In another embodiment, polyphenol antioxidant dosage of the present invention ranges from 100-1500 mg/day. In another embodiment, polyphenol antioxidant dosage of the present invention ranges from 100-1000 mg/day. In another embodiment, polyphenol antioxidant dosage of the present invention ranges from 100-800 mg/day. In another embodiment, polyphenol antioxidant dosage of the present invention ranges from 150-600 mg/day. Each possibility represents a separate embodiment of the present invention.

In another embodiment, the antioxidant of the present invention is a nonflavonoid phenolic antioxidant. In another embodiment, the nonflavonoid phenolic antioxidant is curcumin. In another embodiment, the nonflavonoid phenolic antioxidant is xanthone. In another embodiment, the nonflavonoid phenolic antioxidant is silymarin. In another embodiment, the nonflavonoid phenolic antioxidant is eugenol. Each possibility represents a separate embodiment of the present invention.

In another embodiment, a nonflavonoid phenolic antioxidant of the present invention is formulated in a gelatin capsule. In another embodiment, a nonflavonoid phenolic antioxidant of the present invention is formulated as tablets. In another embodiment, a nonflavonoid phenolic antioxidant of the present invention is formulated as chewable tablets. Each possibility represents a separate embodiment of the present invention.

In another embodiment, curcumin of the present invention is obtained from curry spice. In another embodiment, curcumin of the present invention is synthetic curcumin. In another embodiment, xanthone of the present invention is obtained by heating phenyl salicylate. In another embodiment, silymarin of the present invention is obtained from Silybum marianum. In another embodiment, silymarin of the present invention is synthetic silymarin. In another embodiment, silymarin of the present invention is a complex of silymarin and phosphatidylcholine (lecithin). In another embodiment, eugenol of the present invention is obtained from clove. In another embodiment, eugenol of the present invention is synthetic eugenol. Each possibility represents a separate embodiment of the present invention.

In some embodiments, nonflavonoid phenolic antioxidant dosage of the present invention ranges from 1-3000 mg/day. In another embodiment, nonflavonoid phenolic antioxidant dosage of the present invention ranges from 10-3000 mg/day. In another embodiment, nonflavonoid phenolic antioxidant dosage of the present invention ranges from 10-2500 mg/day. In another embodiment, nonflavonoid phenolic antioxidant dosage of the present invention ranges from 100-2500 mg/day. In another embodiment, nonflavonoid phenolic antioxidant dosage of the present invention ranges from 100-2000 mg/day. In another embodiment, nonflavonoid phenolic antioxidant dosage of the present invention ranges from 150-2000 mg/day. In another embodiment, nonflavonoid phenolic antioxidant dosage of the present invention ranges from 200-2000 mg/day. In another embodiment, nonflavonoid phenolic antioxidant dosage of the present invention ranges from 200-1500 mg/day. In another embodiment, nonflavonoid phenolic antioxidant dosage of the present invention ranges from 400-1500 mg/day. Each possibility represents a separate embodiment of the present invention.

In another embodiment, the antioxidant of the present invention is citric acid. In another embodiment, citric acid of the present invention is formulated in a gelatin capsule. In another embodiment, citric acid of the present invention is formulated as tablets. In another embodiment, citric acid of the present invention is formulated as chewable tablets. Each possibility represents a separate embodiment of the present invention.

In another embodiment, citric acid of the present invention is obtained from lemons. In another embodiment, citric acid of the present invention is obtained from limes. In another embodiment, citric acid is produced using cultures of Aspergillus Niger fed on sucrose to produce citric acid. In another embodiment, citric acid is isolated by precipitating it with calcium hydroxide to yield calcium citrate salt. In another embodiment, citric acid is regenerated by treatment with sulfuric acid. In another embodiment, citric acid is isolated from the fermentation broth by extraction with a hydrocarbon solution of the organic base trilaurylamine followed by re-extraction from the organic solution by water. Each possibility represents a separate embodiment of the present invention.

In some embodiments, citric acid dosage of the present invention ranges from 1-8000 mg/day. In another embodiment, citric acid dosage of the present invention ranges from 10-6000 mg/day. In another embodiment, citric acid dosage of the present invention ranges from 20-6000 mg/day. In another embodiment, citric acid dosage of the present invention ranges from 50-5000 mg/day. In another embodiment, citric acid dosage of the present invention ranges from 100-5000 mg/day. In another embodiment, citric acid dosage of the present invention ranges from 150-6000 mg/day. In another embodiment, citric acid dosage of the present invention ranges from 200-6000 mg/day. In another embodiment, citric acid dosage of the present invention ranges from 300-8000 mg/day. In another embodiment, citric acid dosage of the present invention ranges from 250-4000 mg/day. In another embodiment, citric acid dosage of the present invention ranges from 250-3000 mg/day. In another embodiment, citric acid dosage of the present invention ranges from 300-4000 mg/day. In another embodiment, citric acid dosage of the present invention ranges from 400-3000 mg/day. Each possibility represents a separate embodiment of the present invention.

In another embodiment, the antioxidant of the present invention is oxalic acid. In another embodiment, oxalic acid of the present invention is formulated in a gelatin capsule. In another embodiment, oxalic acid of the present invention is formulated as tablets. In another embodiment, oxalic acid of the present invention is formulated as chewable tablets. Each possibility represents a separate embodiment of the present invention.

In another embodiment, oxalic acid of the present invention is obtained from fat hen. In another embodiment, oxalic acid of the present invention is obtained from sorrels. In another embodiment, oxalic acid of the present invention is obtained from leaves of tea. In another embodiment, oxalic acid of the present invention is obtained from rhubarbs. In another embodiment, oxalic acid of the present invention is obtained from buckwheats. In another embodiment, oxalic acid of the present invention is obtained from star fruit. In another embodiment, oxalic acid of the present invention is obtained from black pepper. In another embodiment, oxalic acid of the present invention is obtained from parsley. In another embodiment, oxalic acid of the present invention is obtained from poppy seed. In another embodiment, oxalic acid of the present invention is obtained from amaranth. In another embodiment, oxalic acid of the present invention is obtained from spinach. In another embodiment, oxalic acid of the present invention is obtained from chard. In another embodiment, oxalic acid of the present invention is obtained from cocoa. In another embodiment, oxalic acid of the present invention is obtained from berries. In another embodiment, oxalic acid of the present invention is obtained from beans. In another embodiment, oxalic acid is prepared by oxidizing sucrose using nitric acid as the oxidizer and a small amount of vanadium pentoxide as a catalyst. In another embodiment, sodium oxalate is manufactured by absorbing carbon monoxide under pressure in hot sodium hydroxide. Each possibility represents a separate embodiment of the present invention.

In some embodiments, oxalic acid dosage of the present invention ranges from 1-9000 mg/day. In another embodiment, oxalic acid dosage of the present invention ranges from 10-8000 mg/day. In another embodiment, oxalic acid dosage of the present invention ranges from 20-8000 mg/day. In another embodiment, oxalic acid dosage of the present invention ranges from 50-6000 mg/day. In another embodiment, oxalic acid dosage of the present invention ranges from 100-5000 mg/day. In another embodiment, oxalic acid dosage of the present invention ranges from 150-6000 mg/day. In another embodiment, oxalic acid dosage of the present invention ranges from 150-5000 mg/day. In another embodiment, oxalic acid dosage of the present invention ranges from 200-7000 mg/day. In another embodiment, oxalic acid dosage of the present invention ranges from 200-4000 mg/day. In another embodiment, oxalic acid dosage of the present invention ranges from 400-2000 mg/day. In another embodiment, oxalic acid dosage of the present invention ranges from 200-1500 mg/day. Each possibility represents a separate embodiment of the present invention.

In another embodiment, the antioxidant of the present invention is phytic acid. In another embodiment, phytic acid of the present invention is formulated in a gelatin capsule. In another embodiment, phytic acid of the present invention is formulated as tablets. In another embodiment, phytic acid of the present invention is formulated as chewable tablets.

In another embodiment, phytic acid of the present invention is obtained from hulls. In another embodiment, phytic acid of the present invention is obtained from of nuts. In another embodiment, phytic acid of the present invention is obtained from seeds. In another embodiment, phytic acid of the present invention is obtained from grains. In another embodiment, phytic acid of the present invention is synthetic phytic acid. Each possibility represents a separate embodiment of the present invention.

In some embodiments, phytic acid dosage of the present invention ranges from 1-8000 mg/day. In another embodiment, phytic acid dosage of the present invention ranges from 100-6000 mg/day. In another embodiment, phytic acid dosage of the present invention ranges from 100-6000 mg/day. In another embodiment, phytic acid dosage of the present invention ranges from 200-6000 mg/day. In another embodiment, phytic acid dosage of the present invention ranges from 500-6000 mg/day. In another embodiment, phytic acid dosage of the present invention ranges from 800-6000 mg/day. In another embodiment, phytic acid dosage of the present invention ranges from 800-3000 mg/day. In another embodiment, phytic acid dosage of the present invention ranges from 1000-6000 mg/day. In another embodiment, phytic acid dosage of the present invention ranges from 1500-4000 mg/day. Each possibility represents a separate embodiment of the present invention.

In another embodiment, the antioxidant of the present invention is lignan. In another embodiment, lignan of the present invention is formulated in a gelatin capsule. In another embodiment, lignan of the present invention is formulated as tablets. In another embodiment, lignan of the present invention is formulated as chewable tablets. Each possibility represents a separate embodiment of the present invention.

In another embodiment, lignan of the present invention is obtained from flax seed. In another embodiment, lignan of the present invention is obtained from of pumpkin seeds. In another embodiment, lignan of the present invention is obtained from sesame seeds. In another embodiment, lignan of the present invention is obtained from rye. In another embodiment, lignan of the present invention is obtained from soybeans. In another embodiment, lignan of the present invention is obtained from broccoli. In another embodiment, lignan of the present invention is obtained from beans. In another embodiment, lignan of the present invention is obtained from berries. In another embodiment, lignan of the present invention is synthetic lignan. Each possibility represents a separate embodiment of the present invention.

In some embodiments, lignan dosage of the present invention ranges from 1-10000 mg/day. In another embodiment, lignan dosage of the present invention ranges from 100-10000 mg/day. In another embodiment, lignan dosage of the present invention ranges from 100-8000 mg/day. In another embodiment, lignan dosage of the present invention ranges from 250-8000 mg/day. In another embodiment, lignan dosage of the present invention ranges from 1000-8000 mg/day. In another embodiment, lignan dosage of the present invention ranges from 1000-4000 mg/day. In another embodiment, lignan dosage of the present invention ranges from 1200-6000 mg/day. In another embodiment, lignan dosage of the present invention ranges from 200-5000 mg/day. In another embodiment, lignan dosage of the present invention ranges from 2000-5000 mg/day. In another embodiment, lignan dosage of the present invention ranges from 1000-3000 mg/day. Each possibility represents a separate embodiment of the present invention.

In another embodiment, the antioxidant of the present invention is bilirubin. In another embodiment, bilirubin of the present invention is formulated in a gelatin capsule. In another embodiment, bilirubin of the present invention is formulated as tablets. In another embodiment, bilirubin of the present invention is formulated as chewable tablets. In another embodiment, bilirubin of the present invention is formulated in a solution.

In another embodiment, bilirubin of the present invention is obtained from an animal source. In another embodiment, bilirubin of the present invention is obtained from a ma μM al. In another embodiment, bilirubin of the present invention is synthetic bilirubin. Each possibility represents a separate embodiment of the present invention.

In some embodiments, bilirubin dosage of the present invention ranges from 1-5000 mg/day. In another embodiment, bilirubin dosage of the present invention ranges from 10-3000 mg/day. In another embodiment, bilirubin dosage of the present invention ranges from 10-2000 mg/day. In another embodiment, bilirubin dosage of the present invention ranges from 50-2000 mg/day. In another embodiment, bilirubin dosage of the present invention ranges from 100-2000 mg/day. In another embodiment, bilirubin dosage of the present invention ranges from 250-2000 mg/day. In another embodiment, bilirubin dosage of the present invention ranges from 500-2000 mg/day. In another embodiment, bilirubin dosage of the present invention ranges from 1000-2000 mg/day. In another embodiment, bilirubin dosage of the present invention ranges from 700-1500 mg/day. In another embodiment, bilirubin dosage of the present invention ranges from 800-1200 mg/day. In another embodiment, bilirubin dosage of the present invention ranges from 800-1800 mg/day. Each possibility represents a separate embodiment of the present invention.

In another embodiment, the antioxidant of the present invention is uric acid. In another embodiment, uric acid of the present invention is formulated in a gelatin capsule. In another embodiment, uric acid of the present invention is formulated as tablets. In another embodiment, uric acid of the present invention is formulated as chewable tablets. In another embodiment, uric acid of the present invention is formulated in an oily solution. Each possibility represents a separate embodiment of the present invention.

In another embodiment, uric acid of the present invention is obtained from an animal source. In another embodiment, uric acid of the present invention is obtained from a ma μM al. In another embodiment, uric acid of the present invention is synthetic uric acid. Each possibility represents a separate embodiment of the present invention.

In some embodiments, uric acid dosage of the present invention ranges from 1-8000 mg/day. In another embodiment, uric acid dosage of the present invention ranges from 10-8000 mg/day. In another embodiment, uric acid dosage of the present invention ranges from 100-8000 mg/day. In another embodiment, uric acid dosage of the present invention ranges from 500-8000 mg/day. In another embodiment, uric acid dosage of the present invention ranges from 700-8000 mg/day. In another embodiment, uric acid dosage of the present invention ranges from 1000-8000 mg/day. In another embodiment, uric acid dosage of the present invention ranges from 1000-6000 mg/day. In another embodiment, uric acid dosage of the present invention ranges from 1000-4000 mg/day. In another embodiment, uric acid dosage of the present invention ranges from 500-3000 mg/day. In another embodiment, uric acid dosage of the present invention ranges from 1200-6000 mg/day. In another embodiment, uric acid dosage of the present invention ranges from 2000-4000 mg/day. In another embodiment, uric acid dosage of the present invention ranges from 2000-3000 mg/day. Each possibility represents a separate embodiment of the present invention.

In another embodiment, the antioxidant of the present invention is lipoic acid. In another embodiment, lipoic acid of the present invention is α-Lipoic acid. In another embodiment, α-Lipoic acid of the present invention is R-α-Lipoic acid. In another embodiment, lipoic acid of the present invention is formulated in a gelatin capsule. In another embodiment, lipoic acid of the present invention is formulated as tablets. In another embodiment, lipoic acid of the present invention is formulated as chewable tablets. In another embodiment, lipoic acid of the present invention is formulated in a solution. Each possibility represents a separate embodiment of the present invention.

In another embodiment, lipoic acid of the present invention is obtained from an animal source. In another embodiment, lipoic acid of the present invention is obtained from a ma μM al. In another embodiment, lipoic acid of the present invention is obtained from a kidney. In another embodiment, lipoic acid of the present invention is obtained from a heart. In another embodiment, lipoic acid of the present invention is obtained from a liver. In another embodiment, lipoic acid of the present invention is obtained from spinach. In another embodiment, lipoic acid of the present invention is obtained from broccoli. In another embodiment, lipoic acid of the present invention is obtained from potatoes. In another embodiment, lipoic acid of the present invention is synthetic lipoic acid. Each possibility represents a separate embodiment of the present invention.

In some embodiments, lipoic acid dosage of the present invention ranges from 1-6000 mg/day. In another embodiment, lipoic acid dosage of the present invention ranges from 10-6000 mg/day. In another embodiment, lipoic acid dosage of the present invention ranges from 100-6000 mg/day. In another embodiment, lipoic acid dosage of the present invention ranges from 250-6000 mg/day. In another embodiment, lipoic acid dosage of the present invention ranges from 500-6000 mg/day. In another embodiment, lipoic acid dosage of the present invention ranges from 750-6000 mg/day. In another embodiment, lipoic acid dosage of the present invention ranges from 1000-6000 mg/day. In another embodiment, lipoic acid dosage of the present invention ranges from 1000-4000 mg/day. In another embodiment, lipoic acid dosage of the present invention ranges from 1200-4000 mg/day. In another embodiment, lipoic acid dosage of the present invention ranges from 1500-3000 mg/day. In another embodiment, lipoic acid dosage of the present invention ranges from 2000-4000 mg/day. In another embodiment, lipoic acid dosage of the present invention ranges from 800-2500 mg/day. Each possibility represents a separate embodiment of the present invention.

In another embodiment, the antioxidant of the present invention is N-Acetylcysteine. In another embodiment, N-Acetylcysteine of the present invention is formulated in a gelatin capsule. In another embodiment, N-Acetylcysteine of the present invention is formulated as tablets. In another embodiment, N-Acetylcysteine of the present invention is formulated as chewable tablets. In another embodiment, N-Acetylcysteine of the present invention is formulated in a solution. Each possibility represents a separate embodiment of the present invention.

In another embodiment, N-Acetylcysteine of the present invention is derived from the amino acid Cysteine.

In some embodiments, N-Acetylcysteine of the present invention ranges from 1-25000 mg/day. In another embodiment, N-Acetylcysteine dosage of the present invention ranges from 500-10000 mg/day. In another embodiment, N-Acetylcysteine dosage of the present invention ranges from 1000-20000 mg/day. In another embodiment, N-Acetylcysteine dosage of the present invention ranges from 1000-20000 mg/day. In another embodiment, N-Acetylcysteine dosage of the present invention ranges from 1000-15000 mg/day. In another embodiment, N-Acetylcysteine of the present invention ranges from 1500-9000 mg/day. In another embodiment, N-Acetylcysteine dosage of the present invention ranges from 1500-8000 mg/day. In another embodiment, N-Acetylcysteine dosage of the present invention ranges from 1500-6000 mg/day. In another embodiment, N-Acetylcysteine dosage of the present invention ranges from 1000-10000 mg/day. In another embodiment, N-Acetylcysteine dosage of the present invention ranges from 2000-8000 mg/day. In another embodiment, N-Acetylcysteine dosage of the present invention ranges from 2500-6500 mg/day. In another embodiment, N-Acetylcysteine dosage of the present invention ranges from 1500-3000 mg/day. Each possibility represents a separate embodiment of the present invention.

In another embodiment, the antioxidant of the present invention is ebselen [2-phenyl-1,2-benzisoselenazol-3(2H)-one], a lipid-soluble seleno-organic compound. In another embodiment, ebselen of the present invention is formulated in a gelatin capsule. In another embodiment, ebselen of the present invention is formulated as tablets. In another embodiment, ebselen of the present invention is formulated as chewable tablets. In another embodiment, ebselen of the present invention is formulated in a solution.

In some embodiments, ebselen daily dosage of the present invention ranges from 100-15000 mg/day. In another embodiment, ebselen daily dosage of the present invention ranges from 500-15000 mg/day. In another embodiment, ebselen daily dosage of the present invention ranges from 1000-15000 mg/day. In another embodiment, ebselen daily dosage of the present invention ranges from 1000-12000 mg/day. In another embodiment, ebselen daily dosage of the present invention ranges from 1000-10000 mg/day. In another embodiment, ebselen daily dosage of the present invention ranges from 1500-8000 mg/day. In another embodiment, ebselen daily dosage of the present invention ranges from 2000-8000 mg/day. In another embodiment, ebselen daily dosage of the present invention ranges from 1500-6000 mg/day. In another embodiment, ebselen daily dosage of the present invention ranges from 1500-3000 mg/day. In another embodiment, ebselen daily dosage of the present invention ranges from 2000-6000 mg/day. In another embodiment, ebselen daily dosage of the present invention ranges from 500-2500 mg/day. Each possibility represents a separate embodiment of the present invention.

In another embodiment, the antioxidant of the present invention is idebenone [Cas no.: 58186-27-9]. In another embodiment, idebenone of the present invention is in a crystalline form. In another embodiment, idebenone of the present invention is in a powder form. In another embodiment, idebenone of the present invention is formulated in a gelatin capsule. In another embodiment, idebenone of the present invention is formulated as tablets. In another embodiment, idebenone of the present invention is formulated as chewable tablets. In another embodiment, idebenone of the present invention is formulated in a solution. Each possibility represents a separate embodiment of the present invention.

In some embodiments, idebenone daily dosage of the present invention ranges from 10-3000 mg/day. In another embodiment, idebenone daily dosage of the present invention ranges from 10-3000 mg/day. In another embodiment, idebenone daily dosage of the present invention ranges from 30-3000 mg/day. In another embodiment, idebenone daily dosage of the present invention ranges from 30-2000 mg/day. In another embodiment, idebenone daily dosage of the present invention ranges from 30-1000 mg/day. In another embodiment, idebenone daily dosage of the present invention ranges from 30-800 mg/day. In another embodiment, idebenone daily dosage of the present invention ranges from 30-600 mg/day. In another embodiment, idebenone daily dosage of the present invention ranges from 100-400 mg/day. In another embodiment, idebenone daily dosage of the present invention ranges from 30-200 mg/day. In another embodiment, idebenone daily dosage of the present invention ranges from 100-300 mg/day. In another embodiment, idebenone daily dosage of the present invention ranges from 200-600 mg/day. In another embodiment, idebenone daily dosage of the present invention ranges from 500-1000 mg/day. Each possibility represents a separate embodiment of the present invention.

In another embodiment, the present invention provides a kit comprising a compound or composition utilized in performing a method of the present invention. In another embodiment, the present invention provides a kit comprising a composition, tool, or instrument of the present invention. Each possibility represents a separate embodiment of the present invention. Each possibility represents a separate embodiment of the present invention.

In another embodiment, additional methods of administering the antioxidant compounds of the present invention comprise injectable dosage forms. In another embodiment, the injectable is administered intraperitonealy. In another embodiment, the injectable is administered intramuscularly. In another embodiment, the injectable is administered intradermally. In another embodiment, the injectable is administered intravenously. Each possibility represents a separate embodiment of the present invention.

In another embodiment, additional methods of administering the antioxidant compounds of the present invention comprise solutions. In another embodiment, the solution is administered orally. In another embodiment, the solution is administered by infusion. In another embodiment, the solution is a solution for inhalation. Each possibility represents a separate embodiment of the present invention.

In another embodiment, the antioxidant compounds of the present invention are administered throughout the course of the disease. In another embodiment, the compound is administered during symptomatic stages of the disease. In another embodiment, the compound is administered as a pre-treatment for prevention of the disease. In another embodiment, the compound is administered as a post-treatment for preventing relapse of the disease. Each possibility represents a separate embodiment of the present invention. Each possibility represents a separate embodiment of the present invention.

Combinations

In some embodiments, the methods of the present invention comprise compositions that comprise more than a compound of the invention. In another embodiment, the compositions of the present invention are formulated as multi-antioxidant dosage forms. In another embodiment, the compositions of the present invention are formulated as multi-vitamin dosage forms. In some embodiments, the compositions of the present invention will comprise at lease one antioxidant of the invention, in any form or embodiment as described herein. In some embodiments, the term “comprise” refers to the inclusion of the indicated antioxidant compound, as well as inclusion of other active agents, and pharmaceutically acceptable carriers, excipients, emollients, stabilizers, etc., as are known in the pharmaceutical industry. Each possibility represents a separate embodiment of the present invention.

In another embodiment, the present invention provides combined preparations. In another embodiment, the term “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In another embodiment, the combined preparation can be varied, e.g., in order to cope with the needs of a patient subpopulation to be treated or the needs of the single patient which different needs can be due to the severity of the disease, age, sex, or body weight as can be readily made by a person skilled in the art. Each possibility represents a separate embodiment of the present invention.

Various embodiments of dosage ranges are contemplated by this invention. The dosage of the multi-antioxidant formulations of the present invention may be in the range of 0.1-10000 mg/day. In another embodiment, the dosage is in the range of 1-10000 mg/day. In another embodiment, the dosage is in the range of 50-10000 mg/day. In another embodiment, the dosage is in the range of 100-10000 mg/day. In another embodiment, the dosage is in the range of 500-10000 mg/day. In another embodiment, the dosage is in the range of 500-8000 mg/day. In another embodiment, the dosage is in the range of 300-6000 mg/day. In another embodiment, the dosage is in the range of 300-5000 mg/day. In another embodiment, the dosage is in the range of 300-4500 mg/day. In another embodiment, the dosage is in the range of 350-4500 mg/day. In another embodiment, the dosage is in the range of 400-4500 mg/day. In another embodiment, the dosage is in the range of 400-4000 mg/day. In another embodiment, the dosage is in a range of 450-4000 mg/day. In another embodiment, the dosage is in the range of 500-4000 mg/day. In another embodiment, the dosage is in a range of 500-3500 mg/day. In another embodiment, the dosage is in the range of 500-3000 mg/day. Each possibility represents a separate embodiment of the present invention.

In another embodiment, the methods of the present invention comprise compositions that comprise a compound of the invention combined with another SMA treatment. In another embodiment, the methods of the present invention comprise compositions that comprise a compound of the invention combined with an agent that induces SMN protein expression. In another embodiment, the methods of the present invention comprise compositions that comprise a compound of the invention combined with a Histone deacetylase inhibitor. In another embodiment, the methods of the present invention comprise compositions that comprise a compound of the invention combined with indoprofen analogs that increase SMN protein expression. In another embodiment, the methods of the present invention comprise compositions that comprise a compound of the invention combined with phenylbutyrate. In another embodiment, the methods of the present invention comprise compositions that comprise a compound of the invention combined with interferon beta. In another embodiment, the methods of the present invention comprise compositions that comprise a compound of the invention combined with interferon gamma. In another embodiment, the methods of the present invention comprise compositions that comprise a compound of the invention combined with Trichostatin A.

In another embodiment, the methods of the present invention comprise compositions that comprise a compound of the invention combined with a compound selected from: 1-Boc-homopiperazine, 2-(4-methyl010cyclohexylidene), acetic acid, 2-N-Butylthiophene, 3,4-Dimethoxyphenylacetone, 4-Pentenoic acid, 5-10-Dihydro-5-10-dimethylphenazine, Acetrizoate Allyl Disulfide, alpha-Bromohepyanoic acid, Altretamine, Alosetron, Amantadine, Amikacin, Amrinone, Anisotropine, Methylbromide, Ascorbic Acid, Azlocillin, Beclomethasone, Benfluorex, Benzyl Benzoate Beta-Ionol, Betaxolol, Bethanechol, Chloride Bezafibrate, Bis(2-Ethlyhexyl), Fumarate, Boldine, Busulfan, Calcitonin, Calcium Chloride, Carbenicillin, Chlophedianol, Chlortetracycline, Chymopapain Cinoxacin, Citronellal, Creatine, Cupric, Chloride, Cyclosporine, Desoxycorticosterone Acetate, Dibekacin, Divalproex, dl-Penicillamine, Dobutamine, Efavirenz, Ellipticine, Enalapril, Epinephrine, Ethionamide, Ethopropazine, Fenpiverinium, Bromide, Ferric A μM onium Citrate, Ferrous Sulfate, Fluocinonide, Fosfosal, Framycetin, Gabapentin, Gadoteridol, Gallamine, Triethiodide, Gentamicin, Geranyl Acetate, Glutathione Disulfide, Guanethidine, Guanfacine H-8, Hexamethyltetracosane, Indoprofen, Isopulegol acetate, Khellin Lactitol, Levetiracetam L-Methionine, Sulfoximine, Loxapine L-Tartaric acid, Manganese Sulfate, Mastic Memantine, Menthol, Metampicillin, Methyl Linoleate, Methyl Oleate, Methyl Palmitoleate, Moxisylyte, Neomycin, Nifuroxazide, Nonoxynol-9, Oxaceprol, Oxprenolol, Pargyline, Paromomycin, Pazufloxacin, Pentagastrin, Pergolide, piperazine, Propranolol, Pyrantel, Pyridostigmine Bromide, Rescinnamine, S-Allyl-L-Cysteine, Selenium Sulfide, Serotonin, Sirtinol Spectinomycin, Tegafur, Teicoplanin, Tiapride, Trans-Anethole, Ubenimex, Ursodiol, or Vincamine.

In another embodiment, the methods of the present invention comprise compositions that comprise a compound of the invention combined with a compound selected from selegiline; 4-Cl-kynurenine; A-134974; A-366833; A-35380; A-72055; ABS-205; AC-184897; AC-90222; ACEA-1021 (licostinel); ADCI; AEG-3482; AGY-110; AGY-207; AK-275 (vasolex); alaptid; ALE-0540; AM-36; annovis; ampakines; amyloid-inhibiting peptides; AN-1792; andrographolide; APBPI-124; apoptosin; aptiganel; AR-139525; AR-15896 (lanicemine); AR-A-008055; donepezil; AR-R-17779; AR-R18565; ARRY-142886; ARX-2000; ARX-2001; ARX-2002; AS-600292; AS-004509; AS-601245; autovac; axokine; AZ-36041; BA-1016; Bay Q 3111 (BAY-X-9227; N-(2-ethoxyphenyl)-N′-(1,2,3-trimethylpropyl)-2-nitroethene-1,1-diamine); BD-1054; BGC-20-1178; BIMU-8 ((endo-N-8-methyl-8-azabicyclo-(3.2.1)oct-3-yl)-2,3-dihydro-3-isopropyl-2-oxo-1H-benzimidazol-1-carboxamide); BLS-602; BLS-605; BMS-181100 (alpha-(4-fluorophenyl)-4-(5-fluoro-2-pyrimidinyl)-1-piperazine butanol); brasofensine; breflate; BTG-A derivatives; C60 fullerenes; CAS-493 (aloracetam); celecoxib; CEP-1347; CEP-3122; CEP-4143; CEP-4186; CEP-751; CERE-20; CGP-35348 (P-(3-aminopropyl)-P-diethoxymethylphosphinic acid); CHF-2060; CNIC-568; CNS-1044; CNS-2103; CNS-5065; coenzyme Q10; CP-132484 (1-(2-aminoethyl)-3-methyl-8,9-dihydropyrano(3,2-e)indole); CP-283097; CPC-304; CX-516; cyclophosphamide; cyclosporin A; dabelotine; DCG-IV (2-(2,3-dicarboxycyclopropyl)glycine); DD-20207; dehydroascorbic acid; dexanabinol; dexefaroxan; dihydroquinolines; diperdipine; dizocilpine; DMP-543; DP-103; DP-109; DP-b99; DPP-225; dykellic acid; E-2101; EAA-404 (midafotel); EAB-318; edaravone; EF-7412; EGIS-7444; EHT-202; eliprodil; emopamil; EP-475; EQA-00 (anapsos); ES-242-1; estrogen or estrogen/progesterone; ethanoanthracene derivatives; F-10981; F-2-CCG-I; FCE-29484A; FCE-29642A; FGF-9; FGF-16; ersofermin; formobactin; FPL-16283; GAG mimetics; galantamine derivatives; galdansetron; ganstigmine; gavestinel; GDNF (liatermine); GGF-2; GKE-841 (retigabine); glialines (throphix); GM-1 ganglioside; GP-14683; GPI-1337; GPI-1485; GR-73632; GR-89696 (methyl 4-(3,4-dichlorophenyl)acetyl)-3-(1-pyrrolidinylmethyl)-1-piperazinecarbo-xylate fumarate); GSK-3 inhibitors; GT-2342; GT-715; GV-2400; GYKI-52466 (4-(8-methyl-9H-1,3-dioxolo(4,5-h)(2,3)benzodiazepin-5-yl)-benzenamine); HBNF; HF-0220; HP-184 (N-(n-propyl)-3-fluoro-4-pyridinyl-1H-3-methylindol-1-amine hydrochloride); IAPs; IDN-6556; IGF modulators (e.g., neurocrine); igmesine; imidazole derivatives; imidazolyl nitrones; inosine; interferon alpha; interleukin-2-like growth factor; iometopane; ipenoxazone; itameline; KF-17329; KP-102 (alanyl-(2-naphthyl)alanyl-alanyl-tryptophyl-phenylalanyl-lysinamide); KRX-411; KW-6002 (istradefylline; 8-(2-(3,4-dimethoxyphenyl)ethenyl)-1,3-diethyl-3,7-dihydro-7-methyl-1H-pu-rine-2,6-dione); L-687306 (3-(3-cyclopropyl-1,2,4-oxadiazol-5-yl)-1-azabicyclo(2.2.1)heptane); L-687414; L-689560 (trans-2-carboxy-5,7-dichloro-4-(((phenylamino)carbonyl)amino)-1,2,3,4-te-trahydroquinoline); L-701252; lamotrigine; LAU-0501; lazabemide; leteprinim; LIGA-20; LY-178002 (5-((3,5-bis(1,1-dimethylethyl)-4-hydroxyphenyl)methylene)-4-thiazolidino-ne); LY-233536 (decahydro-6-(2H-tetrazol-5-ylmethyl)-3-isoquinolinecarboxylic acid); LY-235959 (decahydro-6-(phosphonomethyl)-3-isoquinolinecarboxylic acid); LY-274614; LY-302427; LY-354006; LY-354740 (2-aminobicyclo(3.1.0)hexane-2,6-dicarboxylic acid); LY-451395; MCC-257; MCI-225 (4-(2-fluorophenyl)-6-methyl-2-(1-piperazinyl)thieno(2,3-d)pyrimi-dine); MDL-100748 (4-((carboxymethyl)amino)-5,7-dichloroquinoline-2-carboxylic acid); MDL-101002; MDL-102288; MDL-105519; MDL-27266 (5-(4-chlorophenyl)-4-ethyl-2,4-dihydro-2-methyl-3H-1,2,4-triazol-3-one); MDL-28170 (carbobenzoxyvalylphenylalanine aldehyde); MDL-29951 (3-(4,6-dichloro-2-carboxyindol-3-yl)propionic acid); mecasermin; MEM-1003; mepindolol; metallotexa-phyrins; methylphenylethynylpyridine (MPEP); microalgal compound; milacemide; mirapex (pramipexole); MLN-519; MS-153; MT-5; N-3393; naltrindole derivatives; NAPVSIPQ; NBI-30702; NC-531; neotrofin; neramexane; nerve growth factor gene therapy; neublastin; neurocalc; neurostrol; NLA-715 (clomethiazole); NNC-07-0775; NNC-07-9202 (2,3-dioxo-6-nitro-7-sulfamoylbenzo(f)quinoxaline); noggin; norleu; NOX-700; NPS-1407; NPS-846; NRT-115; NS-1209; NS-1608 (N-(3-(trifluoromethyl)phenyl)-N′-(2-hydroxy-5-chlorophenyl)urea); NS-2330; NS-257; NS-377; NS-638 (2-amino-1-(4-chlorobenzyl)-5-trifluoromethylbenzimidazole); NS-649; NXD-5150; NXY-059; odapipam; olanzapine; ONO-2506; OPC-14117 (7-hydroxy-1-(4-(3-methoxyphenyl)-1-piperazinyl)acetylamino-2,2,4,6-tetra-methylindan); P-58; P-9939; PACAP; palmidrol; PAN-811; pan-neurotrophin-1; PBT-1 (clioquinol); PD-132026; PD-150606 (3-(4-iodophenyl)-2-mercapto-(Z)-2-propenoic acid); PD-159265; PD-90780; PDC-008.004; PE21; phenserine; philanthotoxins; piperidine derivatives; PK-11195 (1-(2-chlorophenyl)-N-methyl-N-(1-methylpropyl)-3-isoquinolineca-rboxamide); PN-277; PNU-101033E; PNU-157678; PNU-87663; POL-255; posatirelin; PPI-368; PRE-103; propentofylline; protirelin; PRS-211220; PYM-50028; QG-2283; rasagiline; REN-1654; REN-1820; RI-820; riluzole; RJR-1401; Ro-09-2210; rolipram; RPR-104632 (2H-1,2,4-benzothiadiazine-1-dioxide-3-carboxylate acid); RS-100642; S-14820; S-176251; S-34730-1; S-34730; S-18986; S-312-d (methyl 4,7-dihydro-3-isobutyl-6-methyl-4-(nitrophenyl)thieno(2,3-b)-pyridine-5-c-arboxylate); S-33113-1; sabeluzole; safinamide; SB-271046; SB-277011 (trans-N-(4-(2-(6-cyano-1,2,3,4-tetrahydroisoquinolin-2-yl)ethyl)cyclohex-yl)-4-quinolinecarboxamide); SEMAX; SIB-1553A; SIB-1765F (5-ethynyl-3-(1-methyl-2-pyrrolidinyl)pyridine maleate); siclofen; SJA-6017 (N-(4-fluorophenylsulfonyl)-L-valyl-L-leucinal); SKF-74652; SL-34.0026; SLV-308; SNX-482; SP-(V5.2)C; SPC-9766; SPH-1371; SPM-914; SPM-935; SSR-180575; SSR-482073; sumanirole; SUN-05174; survivins; SYM-2207; T-588 (1-(benzo(b)thiophen-5-yl)-2-(2-(N,N-diethylamino)ethoxy)ethanol hydrochloride); tacrine analogs (ABS-301, ABS-302, ABS-304); talampanel; taltirelin; TAN-950A (2-amino-3-(2,5-dihydro-5-oxo-4-isoxazolyl)propanoic acid); TC-2559; TCH-346; TGP-580; thurinex; TK-14; TP-20; traxoprodil; U-74500A (21-(4-(3,6-bis(diethylamino)-2-pyridinyl)-1-piperazinyl)-16-met-hylpregna-1,4,9(11)triene-3,20-dione HCl); U-78517F (2-((4-(2,6-di-1-pyrrolidinyl-4-pyrimidinyl)-1-piperazinyl)methyl)-3,4-di-hydro-2,5,7,8-tetramethyl-2H-1-benzopyran-6-ol.di-HCl); UK-351666; UK-356464; UK-356297; vanoxerine; VX-799; WAY-855; WIB-63480-2; WIN-67500; WIN-68100; WIN-69211; xaliprodene; YM-90K (6-(1H-imidazol-1-yl)-7-nitro-2,3(1H,4H)-quinoxalinedione); ziconotide; and zonampanel.

### SMN Complex

In another embodiment, SMN protein levels parallel the capacity of snRNP assembly (Example 8). In another embodiment, SMN protein levels in a SMA I, SMA II, and SMA III patient cells is considerably reduced. In another embodiment, SMN protein levels in cells exposed to an oxidant is considerably reduced. In another embodiment, reduced snRNP assembly capacity demonstrates a biochemical deficiency in cells.

In some embodiments, complete, or nearly complete, removal or inhibition of the SMN complex results in the inhibition of Sm core assembly in vitro. In another embodiment, there is a linear correlation between the amount of SMN present in the cell extract and the amount of Sm cores that can be formed on specific RNA substrates. In another embodiment, this indicates that the amount of SMN determines the capacity for Sm core assembly. In another embodiment, snRNP assembly is impaired in cells of SMA patients. In another embodiment, snRNP assembly is impaired in GM08333 and GM00498 cells treated with an oxidant.

In another embodiment, the Sm core comprises the seven Sm proteins B/B′, D1, D2, D3, E, F and G bound to the Sm site. In another embodiment, the Sm core is essential for the biogenesis and function of the snRNPs. In another embodiment, the Sm core is required for cap hypermethylation. In another embodiment, the Sm core forms part of the snRNP nuclear localization signal. In another embodiment, the Sm core influences the integration of at least some snRNP-specific proteins into the snRNPs. In another embodiment, the Sm proteins interact with each other strongly and specifically but none of the individual Sm proteins binds stably to RNA. In another embodiment, the Sm RNA-binding site is generated only after the formation of Sm protein heteromers: EFG and D1D2 protein complexes that are minimally required to form a stable intermediate RNP, the so-called sub-core RNP. In another embodiment, Sm core assembly is completed by the subsequent interaction of a B/B′D3 heteromer. In another embodiment, in electron micrographs, all Sm core RNPs possess a similar doughnut-shaped structure.

In another embodiment, the overall amount of the major snRNPs in cells is not reduced. In another embodiment, the strong deficiency in snRNPs rate of accumulation could be detrimental to cells in several ways such as for dividing cells wherein the slower rate of snRNP accumulation could cause a delay in cell cycle progression. In another embodiment, this is the case where the growth rate slows down proportionally to the reduction in SMN. In another embodiment, in non-dividing cells, such as motor neurons, an unmet demand for a timely snRNP production at a particular point in the growth and development of the cell could have severe consequences to the cell. In another embodiment, it could lead to a deficit in functions that depend on an adequate amount of the major snRNPs (e.g., a general decrease in pre-mRNA splicing or an altered processing pattern of pre-mRNA that are required by that cell). In another embodiment, it could lead to a deficit in a specific snRNP (e.g., a RNA of a lower abundance or one that has a lower affinity for the SMN complex). In another embodiment, reduced amounts of SMN complex result in some loss of the regulation of Sm core assembly leading to loss of fidelity of Sm core assembly such that Sm cores assemble on RNAs that are not supposed to receive them. In another embodiment, this is harmful to cells as it interferes with the normal function of these RNAs or causes them to aggregate.

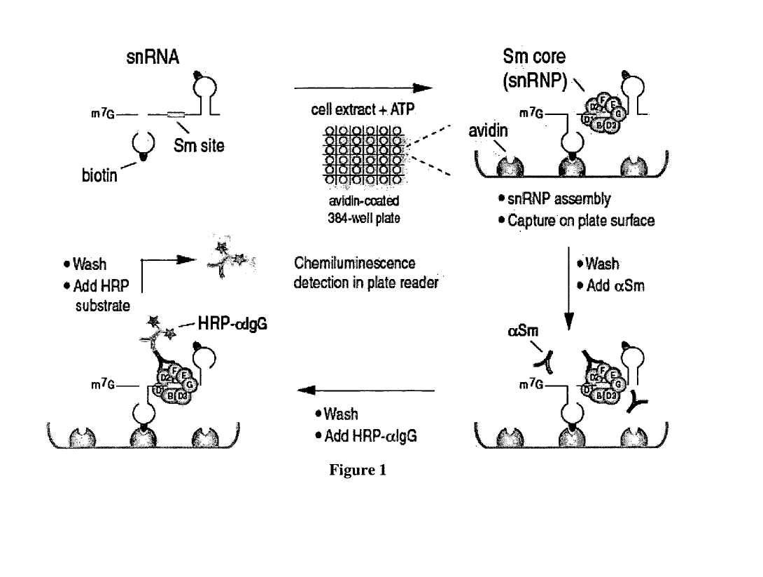

In another embodiment, the present invention comprises the use of a high throughput assay (Example 1, FIG. 1). In another embodiment, the present invention provides that the high throughput assay provides a powerful tool to study the Sm core assembly process. In another embodiment, the present invention provides that the high throughput assay provides a powerful tool to study the mechanism and regulation of the SMN complex. In another embodiment, the high throughput assay is highly sensitive and robust, making it suitable for large-scale screens for chemical and genetic modifiers of the SMN complex. In another embodiment, compounds that modulate the activity of the SMN complex would be useful both as research tools and as potential therapeutics for SMA.

In one embodiment, the high throughput assay described herein, provides a powerful tool to study the Sm core assembly process and the mechanism and regulation of the SMN complex. It is highly sensitive and robust, making it suitable for large-scale screens for chemical and genetic modifiers of the SMN complex. In another embodiment, with slight modifications of the RNA, extract and reaction conditions, this assay can be broadly applied to many other reactions involving RNA-protein interactions. It obviates the need to analyze nucleic acid-protein complexes by gel electrophoretic mobility shifts and opens the way to rapidly identify effectors and dissect the pathway of RNP biogenesis. Although a genetically-determined decrease in functional SMN protein is manifested in loss of motor neurons, the activity of the SMN complex and the biogenesis of snRNPs are of fundamental importance to all eukaryotic cells. Compounds that modulate the activity of the SMN complex would be useful in certain embodiments, both as research tools and as potential therapeutics for SMA. Compounds that increase or bypass the activity of the SMN complex, as well as inhibitors of the SMN complex point in other embodiment to how it is regulated and direct therapeutic approaches in another embodiment. Using the HTS assay, the first class of inhibitors of the SMN complex and of the snRNP biogenesis pathway was identified, and this revealed a surprising and hitherto a novel and unknown aspect of the regulation of the SMN complex

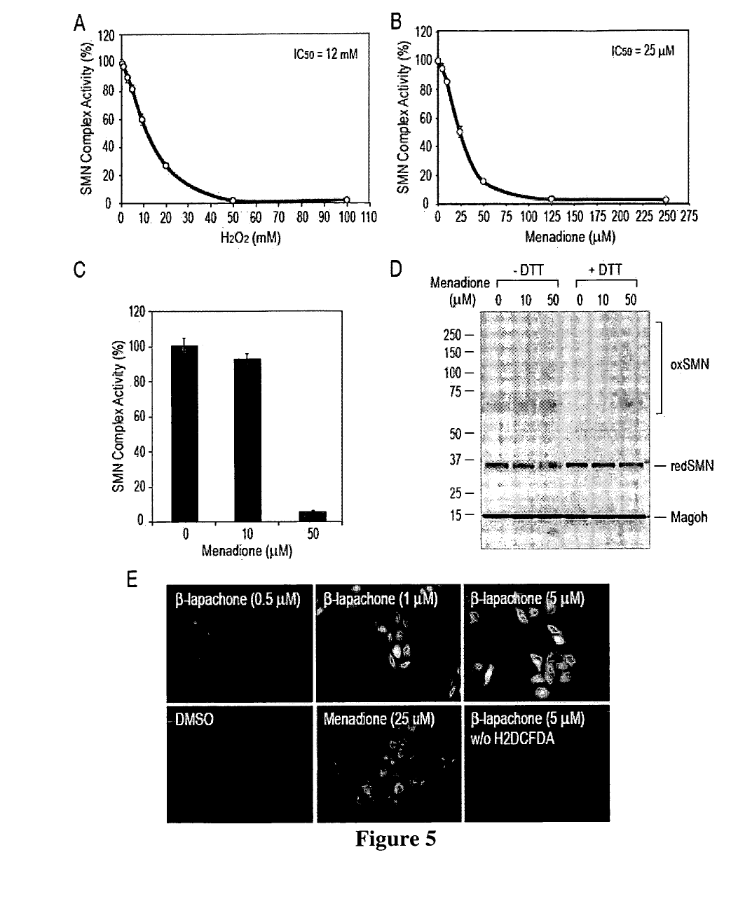

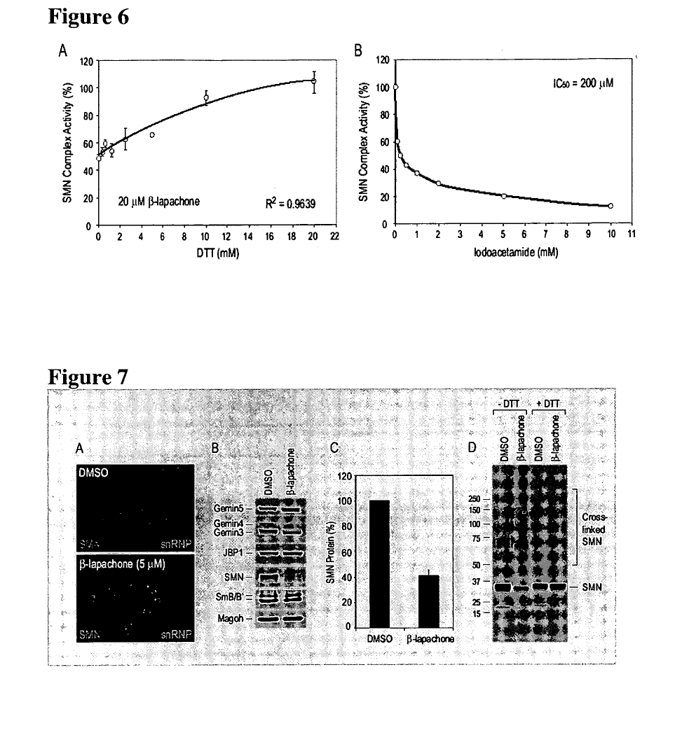

In another embodiment, β-lapachone modulates the activity of the SMN complex. In another embodiment, β-lapachone is derived from lapachol, a naturally occurring naphthoquinone isolated from the Brazilian lapacho tree (Tabebuia avellanedae co μM only known in herbal medicine as Pau D'Arco). In another embodiment, β-lapachone is a potent generator of a futile redox cycle that catalyzes the formation of ROS. In another embodiment, β-lapachone inhibits the SMN complex as a result of ROS it generates. In another embodiment, other structurally-unrelated and diverse oxidants have the same effect. In another embodiment, these include H₂O₂, cumene hydroperoxide and menadione (FIG. 7). In another embodiment, several environmental toxins generate ROS, 9,10-phenanthrenequinone and tetrachloro-1,2-benzoquinone, all of which strongly inhibit the SMN complex. In another embodiment, β-lapachone and other oxidants cause formation of intermolecular disulfide bonds in SMN. In another embodiment, β-lapachone and other oxidants inhibit the activity of the SMN complex. In another embodiment, β-lapachone and other oxidants inhibit the activity of the SMN complex that is reversed by DTT (FIG. 5D and FIG. 6A).

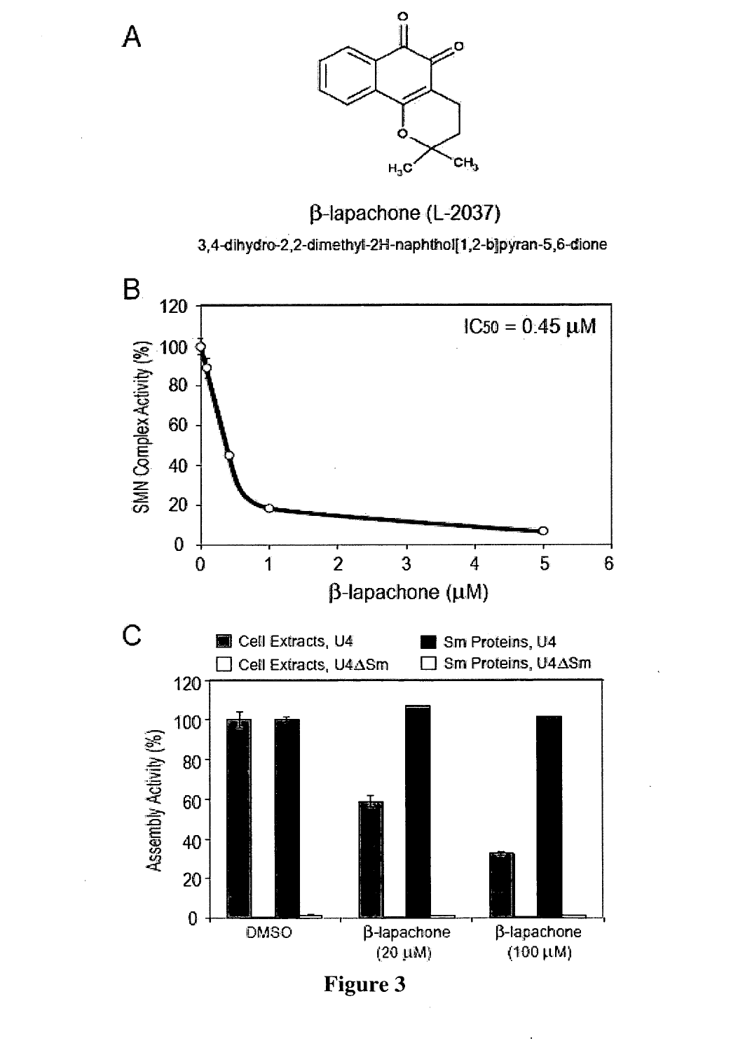

In another embodiment, SMN forms homo-oligomers (FIG. 5D). In another embodiment, human SMN protein contains eight cysteines, several of which are conserved in vertebrates. In another embodiment, the present invention provides that identification of the disulfide crosslinking partners of SMN and the mapping of the cysteines provide important information about its interactions (FIG. 7). In another embodiment, the present invention provides that oxidants provide a powerful new tool to study the structure of the SMN complex. In another embodiment, the SMN complex is readily inactivated by oxidative stress. In another embodiment, other cellular processes involving RNA-protein interactions, including general transcription and translation processes, are not inhibited by oxidation (FIG. 3C).

In one embodiment, the most potent inhibitor, β-lapachone, is a naturally occurring naphthoquinone and a potent generator of a futile redox cycle that catalyzes the formation of ROS. In another embodiment, β-lapachone inhibits several enzymes, including NADH/NADPH oxidoreductase in one embodiment, or topoisomerase I, HIV reverse transcriptase, telomerase and NF-κB in other embodiments. The inhibition of NF-κB by β-lapachone is reversed in another embodiment, by DTT and involves critical sulfhydryl groups. In another embodiment, the inhibition of the SMN complex by β-lapachone is the result of the ROS it generates. In one embodiment, other structurally unrelated and diverse oxidants, including H₂O₂, cumene hydroperoxide as well as several environmental toxins known to generate ROS, menadione, 9,10-phenanthrenequinone and tetrachloro-1,2-benzoquinone, have the same effect (FIG. 5). In another embodiment, the position isomer α-lapachone, which unlike β-lapachone has no appreciable redox cycling capability, does not inhibit the SMN complex. In one embodiment, β-lapachone and the other oxidants cause formation of intermolecular disulfide bonds in SMN and these, as well as the inhibition of the activity of the SMN complex, are counter-acted in another embodiment, or reversed by DTT. In another embodiment, the potency of the compounds used to induce SMN disulfide crosslinks and inactivate the SMN complex parallels their ROS-generating activity in live cells.

In another embodiment, ROS reacts with proteins, DNA and lipids, and can impair a wide range of physiologic functions, cause mutagenesis and elicit apoptosis. In another embodiment, ROS readily react with thiols, including those in protein cysteines, to form sulfenic acid, which in turn reacts readily with available thiols to form disulfides.

In another embodiment, disulfide bonds that form in SMN cysteines, and/or other proteins that are critical for the activity of the SMN complex, could protect it from irreversible damage. In another embodiment, high concentrations of oxidants, β-lapachone or menadione, results in increasing resistance of the inactivation of the SMN complex to reversal by DTT. In another embodiment, forms of reactive nitrogen species (RNS), derived from the multifunctional regulatory molecule nitric oxide (NO), also cause sulfhydryl oxidation in the SMN complex. In another embodiment, SMN complex is highly sensitive to ROS. In another embodiment, different fraction of SMN is oxidized in different cell types. In another embodiment, neurons, including motor neurons are amongst the most metabolically active cells in the body, thus having an extremely high rate of oxygen consumption, and as a consequence, generate relatively high levels of ROS.

In another embodiment, the SMN complex is highly susceptible to ROS. In another embodiment, the role of ROS in neurodegenerative diseases shows a mechanistic convergence of these diseases, particularly amyotrophic lateral sclerosis (ALS) with SMA, with the SMN complex as a plausible target. In another embodiment, antioxidants of the present invention protect the SMN complex from excessive oxidative inactivation.

The sensitivity of the SMN complex to ROS inactivation is remarkable considering that in certain embodiments, transcription, splicing and translation are not inhibited under similar oxidative stress. In one embodiment the disulfide-crosslinked SMN demonstrates that SMN itself becomes oxidized. In another embodiment this is the causative or in another embodiment, the only target of ROS mediated inactivation of the SMN complex. In one embodiment, other proteins that are involved in the snRNP assembly reactions are also modified by ROS, causing inactivation. In another embodiment, ROS provide a powerful tool for studying the structure and domain interactions of SMN. Disulfide bonds can only form if the cysteines involved are in immediate juxtaposition (“zero-distance” crosslinking). The observation of SMN-SMN disulfide crosslinks, indicate that SMN homo-oligomers, previously shown in vitro, exist in cells. These homo-oligomers depend in one embodiment, on sequences encoded in exons 6 and 7, near the carboxyl terminus. The crosslinks of single-cysteine SMNs, C60 and C250, define these cysteines as specific contact points in exon 2b and exon 6, respectively. The absence of C60-C60 contacts in C-terminal deleted SMN indicates that the exon 2b-2b interactions are not sufficient in one embodiment for oligomerization or in another embodiment the C-terminal domain influences the structure of the exon 2b-encoded peptide. Cysteines at positions corresponding to human C60 and C250 are present in many, though not in all vertebrates. In another embodiment, the effect of ROS on the SMN complex in several vertebrate organisms, including mouse and chicken, is similarly inhibited by ROS. In contrast to C60 and C250, the two most highly conserved cysteines, C98 and C123 in human SMN, do not form intermolecular disulfides, consistent with their solvent-inaccessible positions in the interior of the exon 3-encoded Tudor domain.

### Disease

In another embodiment, the present invention provides a method of treating a disease mediated by a deficient spliceosome in a subject, comprising administering to a subject a compound which inhibits SMN protein oxidation. In another embodiment, the methods of the present invention provide a method of treating a disease which involves defects in the spliceosome. In another embodiment, defects in the spliceosome comprise defects in alternative splicing. In another embodiment, the disease is β-thalassemia. In another embodiment, the disease is severe combined immunodeficiency (SCID). In another embodiment, the disease is metachromatic leukodystrophy. In another embodiment, the disease is Menkes Disease. In another embodiment, the disease is Multiple Sclerosis. In another embodiment, the disease is Spinal Muscular Atrophy. In another embodiment, the disease is Adenosine deaminase deficiency. In another embodiment, the disease is Cerebrotendinous xanthomatosis (CTX). In another embodiment, the disease is Sandhoff disease. In another embodiment, the disease is Marfan syndrome. In another embodiment, the disease is Breast cancer. In another embodiment, the disease is ovarian cancer. In another embodiment, the disease is Neurofibromatosis type I. In another embodiment, the disease is acute intermittent porphyria. In another embodiment, the disease is Thrombasthenia of Glanzmann and Naegeli.

In another embodiment, Metachromatic leukodystrophy comprises a disruption in a potential exonic splicing enhancer (ESE) which causes a complete exon 7 skipping. In another embodiment, Metachromatic leukodystrophy comprises nucleotide deletion from the usual exon 8 splice acceptor site of Arylsulfatase A, affecting splice site selection. In another embodiment, G to A mutation is located in the middle of exon 7 and accounts for the disruption in splicing. In another embodiment, C to T substitution, 22 nucleotides downstream from the exon 8 splice acceptor site and accounts for the disruption in splicing.

In another embodiment, Menkes Disease is characterized by skipping of exons 20 and 21 during RNA splicing. In another embodiment, Menkes Disease is an X-linked recessive disorder resulting in a connective-tissue disturbance and profound neurodegeneration in early childhood.

In another embodiment, Protein tyrosine phosphatase receptor type C (PTPRC) disrupted RNA splicing increases the susceptibility to Multiple Sclerosis (MS). In another embodiment, a heterozygous C-to-G transversion at nucleotide 77 of exon 4 of the PTPRC gene prohibits splicing of exon 4 pre-mRNA.

In another embodiment, Spinal Muscular Atrophy is characterized by a mutation which inhibits Exonic splicing enhancers (ESE) within exon 7. In another embodiment, the mutations are caused by deletion within SMN1, or when SMN1 is replaced by nearly identical copy named SMN2 (known as SMNc, SMNcen). In another embodiment, the difference between these two proteins that SMN1 produces the full length protein whereas SMN2 produces truncated, less stable protein, which has a reduced ability to oligomerize. In another embodiment, SMN2 carries a silent mutation in exon 7 (nucleotide transition C is substituted by T). In another embodiment, this silent mutation inhibits Exonic splicing enhancers (ESE) within exon 7 that is ultimately leading to skipping of exon 7.

In another embodiment, Adenosine deaminase deficiency (ADA) is characterized by disrupted splicing caused by skipping of exon 5. In another embodiment, inherited ADA deficiency causes a variable phenotypic spectrum, the most severe being SCID presenting in infancy and usually resulting in early death.

In another embodiment, Cerebrotendinous xanthomatosis (CTX) is characterized by the creation of a cryptic splice site. In another embodiment, CTX is characterized by skipping of the entire exon 2. In another embodiment, exonic silent G-to-T mutation occurs at codon 112, 13 bp upstream from the 3′ terminus of exon 2 in the CYP27A1 gene, which encodes sterol 27-hydroxylase. In another embodiment, silent mutation resulted in alternative pre-mRNA splicing by activating a cryptic 5′ splice site around the mutant codon altsplice.

In another embodiment, Sandhoff disease is characterized by inhibition of normal splicing and decreases in the quantity of mRNA. In another embodiment, Sandhoff disease is characterized by activation of a cryptic splice site. In another embodiment, two mutations (1 in exon and another one in intron) do not affect the splice acceptor consensus sequence or create any new acceptor splice sites, but inhibit the normal splicing and activate the cryptic splice sites. In another embodiment C-to-T transition at +8 of exon 11 (exon 11, +8 CMT) generates predominantly an abnormally spliced mRNA at base +112 of exon 11.

In another embodiment, Marfan syndrome (MFS) is characterized by exonic mutation. In another embodiment, Marfan syndrome (MFS) is characterized by skipping of exon 51, caused by T-G transversion at nucleotide +26 of exon 51. In another embodiment, this mutation creates an amber (TAG) nonsense mutation, substituting a termination codon (X) for a tyrosine (Y) at codon 2113 (Y2113X). In another embodiment, in-frame skipping of FBN1 exon 51 is due to the disruption of an SC35-dependent splicing enhancer within exon 51. In another embodiment, this nonsense mutation induces NMD, which degrades the normally spliced mRNA.

In another embodiment, tumor-necrosis factor receptor superfamily, member 5 (TNFRSF5) is characterized by skipping of exon 5. In another embodiment, skipping of exon 5 is caused by disruption of a putative SF2/ASF binding motif. In another embodiment, homozygous silent mutation at the fifth base pair position of exon 5 occurs in a putative “exonic splicing enhancer”, a cis-element that promotes inclusion of specific exons through binding by the serine/arginine-rich splicing factors, leading to exon skipping and premature termination.

In another embodiment, Breast and Ovarian cancers are characterized by exonic mutation. In another embodiment, mutation in BRCA1, inclusion of exon 18 requires the presence of an intact SF2/ASF-dependent ESE spanning positions +4 to +10. In another embodiment, a natural BRCA1 nonsense mutation at position +6 of exon 18 (E1694X) causes exon skipping.

In another embodiment, Neurofibromatosis type I is characterized by skipping of exons 7, 30, and 37. In another embodiment, mutations within exon 7 were mapped: R304X (910 C-T), Q315X (943 C-T), Q315Q (945 G-A), L316M (946 C-A), W336X (1007 G-A). In another embodiment, adjacent silent and missense mutations were located within highly conserved overlapping stretches of 7 nucleotides with a close similarity to the ESE-specific consensus sequences recognized by the SC35 and SF2/ASF arginine/serine-rich proteins.

In another embodiment, acute intermittent porphyria is characterized by exons skipping and premature translation termination. In another embodiment, three point mutations at the donor splice site of intron 1 result in the activation of a cryptic splice site 67 bp downstream in intron 1. In another embodiment, the cryptic splice site leads to an aberrant exon 1 that in consequence results in a frameshift and finally in a premature translation termination signal at the end of exon 4.

In another embodiment, Thrombasthenia of Glanzmann and Naegeli is characterized by skipping of exon 12. In another embodiment, 11 bp of deletion on the gene GPIIIA occurs in the middle of the exon and results in the change of the reading frame of the GPIIIA mRNA. In another embodiment, Thrombasthenia of Glanzmann and Naegeli is characterized by a mutation in the gene encoding platelet glycoprotein alpha-IIb or the gene encoding platelet glycoprotein IIIa. In another embodiment, Glanzmann thrombasthenia is an autosomal recessive bleeding disorder characterized by failure of platelet aggregation by absent or diminished clot retraction. In another embodiment, the abnormalities are related to quantitative abnormalities of the GPIIb/IIIa platelet surface fibrinogen receptor complex resulting from mutations in either the GPIIb or GPIIIa genes. In another embodiment, the abnormalities are related to qualitative abnormalities of the GPIIb/IIIa platelet surface fibrinogen receptor complex resulting from mutations in either the GPIIb or GPIIIa genes.

In another embodiment, the present invention provides a method of screening compounds altering RNA splicing efficiency in a cell, comprising the step of selective capture of mRNA with a ligand, thereby screening compounds altering splicing efficiency. In another embodiment, RNA splicing modifiers are screened in-vivo for progression or regression. In another embodiment, RNA splicing modifiers are screened in-vitro according to the methods of the present invention.

In another embodiment, the terms assessed, screened, evaluated and analyzed are used interchangeably.

In another embodiment, the invention provides a method of assessing the effect of a protein on RNA splicing efficiency. In another embodiment, the invention provides a method of assessing the effect of a small molecule on RNA splicing efficiency. In another embodiment, the invention provides a method of assessing the effect of an organic molecule on RNA splicing efficiency. In another embodiment, the invention provides a method of assessing the effect of an inorganic molecule on RNA splicing efficiency.

In another embodiment, the invention provides a method of assessing the effect of a therapeutic agent on a primary cell culture derived from a patient suffering from a disease associated with disrupted RNA splicing. In another embodiment, the invention provides a method of assessing the effect of a therapeutic agent on a micro-organ culture derived from a patient suffering from a disease associated with disrupted RNA splicing. In another embodiment, the invention provides a method of assessing the effect of a therapeutic agent on a cell line culture that provides an appropriate model for a given disease which is associated with disrupted RNA splicing. In another embodiment, the in-vivo effect of various agents and conditions is desired.

In another embodiment, the invention provides a method wherein an agent of interest is further administered in-vivo to a human or animal that suffers from a disease of the invention. In another embodiment, administration of an agent is according to procedures known to one skilled in the art. In another embodiment, single or multiple administrations of an agent or agents are required, as known to one skilled in the art. In another embodiment, the agent or agents are administered over a period of days to weeks or over a period of months to years, depending on disease progression and/or regression, as known to one skilled in the art.

In another embodiment, the present invention provides a kit comprising quantitative RNA splice forms measuring reagents. In another embodiment, the present invention provides a kit comprising quantitative mRNA measuring reagents.

In another embodiment, the present invention provides a kit comprising biotinylated ribonucleotides. In another embodiment, the biotinylated ribonucleotides are added to the cell extract. In another embodiment, the biotin tagged spliced RNA is used in affinity chromatography together with a column that has avidin or streptavidine bound to it. In another embodiment, the biotin tagged spliced RNA is used in detection via anti-biotin antibodies or avidine/streptavidine tagged detectors. In another embodiment, tagged detectors comprise but are not limited to horseradish peroxidase, β-galactosidase, alkaline phosphatase, or a green fluorescent protein. In another embodiment, tagged detectors comprise chemiluminescent compounds.

In another embodiment, the present invention provides a kit comprising an exon-exon junction complex (EJC) specific ligand bound to a protein A coated with magnetic beads. In another embodiment, the present invention provides a kit comprising an exon-exon junction complex (EJC) specific ligand bound to a protein G coated with magnetic beads. In another embodiment, the antibody is monoclonal. In another embodiment, the antibody is an Y14 protein specific antibody. In another embodiment, the antibody is a Mago protein specific antibody.

In another embodiment, the present invention provides that SMA results from a reduction in the amount of the full-length SMN protein. In another embodiment, the present invention provides a method of protecting an SMN protein in a subject at risk of developing SMA, comprising the step of administering to a subject a compound which inhibits SMN protein oxidation. In another embodiment, the present invention provides a preventing the development of in a subject at risk of developing SMA, comprising the step of administering to a subject a compound which inhibits SMN protein oxidation. In another embodiment, the present invention provides a method of reducing the risk of de novo mutations is SMA1 thereby protecting an SMN protein in a subject at risk.

In another embodiment, the present invention provides that subject at risk of developing SMA is a subject lacking a single copy of SMN1. In another embodiment, the present invention provides that a subject at risk of developing SMA is a subject lacking a single copy of SMN1 due to conversion of SMN1 to SMN2. In another embodiment, the present invention provides that a subject at risk of developing SMA is a subject lacking a single copy of SMN1 and having a second functional copy of SMN1.

In another embodiment, the present invention provides that protecting the SMA complex from oxidative stress can benefit both the prevention and treatment of SMA by increasing the capacity of the remaining mutant motor neurons to re-innervate skeletal muscle fibers. In another embodiment, the present invention provides that protecting the SMA complex from oxidative stress can contribute to the repair capacity of motor neurons. In another embodiment, the present invention provides that protecting the SMA complex from oxidative stress upregulates SMN2 gene expression, preventing exon 7 skipping of SMN2 transcripts, or stabilizing SMNΔ7. In another embodiment, the present invention provides that protecting the SMA complex from oxidative stress increases the amount of SMN protein encoded by the SMN2 gene by activating the SMN2 gene promoter. In another embodiment, the present invention provides that protecting the SMA complex from oxidative stress increases the amount of SMN protein encoded by the SMN2 gene by preventing the alternative splicing of exon 7.

In another embodiment, the present invention provides that protecting the SMA complex from oxidative have the capacity of self-renewal and differentiation of undifferentiated cells into various cell types including skeletal muscle or neuronal phenotypes.

In another embodiment, the present invention provides that subject at risk of developing SMA is a subject having a hybrid of SMN1/SMN2. In another embodiment, the present invention provides that a subject at risk comprises an SMN exon 7 flanked by SMN1 intron 6 and exon 8. In another embodiment, the present invention provides that a subject at risk comprises an SMN exon 7 flanked by SMN1 intron 6 and exon 8 and SMN1 sequences in all the polymorphic nucleotides except for the SMN2 sequence in exon 8. In another embodiment, the present invention provides that a subject at risk comprises an SMN2 copy near or in the telomeric position. In another embodiment, the present invention provides that a subject at risk comprises increased number of SMN2 copies.

In another embodiment, the present invention provides that a subject at risk of developing SMA is a subject comprising a mutated SMN1. In another embodiment, the present invention provides that a subject at risk of developing SMA is a subject comprising mutated SMN1 comprising small intragenic mutations.

In another embodiment, antioxidants of the present invention prevent the risk of developing SMA in a subject at risk of developing SMA. In another embodiment, antioxidants of the present invention reduce the risk of developing SMA in a subject at risk of developing SMA. In another embodiment, antioxidants of the present invention inhibit the development of SMA in a subject at risk of developing SMA.

In another embodiment, a compound which boosts catalase enzymatic activity of the present invention prevents the risk of developing SMA in a subject at risk of developing SMA. In another embodiment, a compound which boosts catalase enzymatic activity of the present invention reduces the risk of developing SMA in a subject at risk of developing SMA. In another embodiment, a compound which boosts catalase enzymatic activity of the present invention inhibits the development of SMA in a subject at risk of developing SMA.

In another embodiment, a compound which boosts glutathione enzymatic activity of the present invention prevents the risk of developing SMA in a subject at risk of developing SMA. In another embodiment, a compound boosts glutathione enzymatic activity of the present invention reduces the risk of developing SMA in a subject at risk of developing SMA. In another embodiment, a compound which boosts glutathione enzymatic activity of the present invention inhibits the development of SMA in a subject at risk of developing SMA.

In another embodiment, a compound which boosts peroxidase enzymatic activity of the present invention prevents the risk of developing SMA in a subject at risk of developing SMA. In another embodiment, a compound boosts peroxidase enzymatic activity of the present invention reduces the risk of developing SMA in a subject at risk of developing SMA. In another embodiment, a compound which boosts peroxidase enzymatic activity of the present invention inhibits the development of SMA in a subject at risk of developing SMA.

In another embodiment, a compound which boosts SOD enzymatic activity of the present invention prevents the risk of developing SMA in a subject at risk of developing SMA. In another embodiment, a compound boosts SOD enzymatic activity of the present invention reduces the risk of developing SMA in a subject at risk of developing SMA. In another embodiment, a compound which boosts SOD enzymatic activity of the present invention inhibits the development of SMA in a subject at risk of developing SMA.

### Pharmaceutical Compositions and Methods of Administration

“Pharmaceutical composition” refers, in another embodiment, to a therapeutically effective amount of the antioxidant compounds of the present invention, together with a pharmaceutically acceptable carrier or diluent. A “therapeutically effective amount” refers, in another embodiment, to an amount that provides a therapeutic effect for a given condition and administration regimen.

The pharmaceutical compositions containing the antioxidant compounds of the present invention are, in another embodiment, administered to a subject by any method known to a person skilled in the art, such as parenterally, transmucosally, transdermally, intramuscularly, intravenously, intra-dermally, subcutaneously, intra-peritonealy, intra-ventricularly, or intra-cranially. Each possibility represents a separate embodiment of the present invention.

In another embodiment of methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule. Each possibility represents a separate embodiment of the present invention.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, the pharmaceutical compositions are administered topically to body surfaces and are thus formulated in a form suitable for topical administration. Suitable topical formulations include gels, ointments, creams, lotions, drops and the like. For topical administration, the active compound is prepared and applied as solutions, suspensions, or emulsions in a physiologically acceptable diluent with or without a pharmaceutical carrier. Each possibility represents a separate embodiment of the present invention.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of active compound agent over a period of time. Each possibility represents a separate embodiment of the present invention.

In another embodiment, the active compound is delivered in a vesicle, e.g. a liposome.

In other embodiments, carriers or diluents used in methods of the present invention include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs. Each possibility represents a separate embodiment of the present invention.

In another embodiment, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs. Each possibility represents a separate embodiment of the present invention.

In other embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmellose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hydroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants. Each of the above excipients represents a separate embodiment of the present invention. Each possibility represents a separate embodiment of the present invention.

In another embodiment, the pharmaceutical compositions provided herein are controlled-release compositions, i.e. compositions in which the active compound is released over a period of time after administration. Controlled- or sustained-release compositions include formulation in lipophilic depots (e.g. fatty acids, waxes, oils). In another embodiment, the composition is an immediate-release composition, i.e. a composition in which all the active compound is released immediately after administration. Each possibility represents a separate embodiment of the present invention.

In another embodiment, the pharmaceutical composition is delivered in a controlled release system. In another embodiment, the agent is administered using intravenous infusion, an implantable osmotic pump, a transdermal patch, liposomes, or other modes of administration. In another embodiment, a pump is used. In another embodiment, polymeric materials are used; e.g. in microspheres in or an implant. In yet another embodiment, a controlled release system is placed in proximity to the therapeutic target, thus requiring only a fraction of the systemic dose (see, e.g., Goodson, in Medical Applications of Controlled Release, supra, vol. 2, pp. 115-138 (1984); and Langer R, Science 249: 1527-1533 (1990). Each possibility represents a separate embodiment of the present invention.

The compositions also include, in another embodiment, incorporation of the active material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance. Each possibility represents a separate embodiment of the present invention.

Also included in the present invention are particulate compositions coated with polymers (e.g. poloxamers or poloxamines) and the compound coupled to antibodies directed against tissue-specific receptors, ligands or antigens or coupled to ligands of tissue-specific receptors. Each possibility represents a separate embodiment of the present invention.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds. Such modifications also increase, in another embodiment, the compound's solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound. Each possibility represents a separate embodiment of the present invention.

The preparation of pharmaceutical compositions that contain an antioxidant compounds of the present invention are preformed, for example by mixing, granulating, or tablet-forming processes, is well understood in the art. The active therapeutic ingredient is often mixed with excipients that are pharmaceutically acceptable and compatible with the active ingredient. For oral administration, the active compound is mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the active compound is converted into a solution, suspension, or emulsion, if desired with the substances customary and suitable for this purpose, for example, solubilizers or other substances.

Each of the above additives, excipients, formulations and methods of administration represents a separate embodiment of the present invention.

### EXPERIMENTAL DETAILS SECTION

### Materials and Method

### Chemical Compounds

A library of ˜5,000 pure bioactive chemicals that includes FDA-approved drugs, known inhibitors and activators of diverse enzymes and receptors, and pure natural compounds was assembled from several commercial sources (Microsource Diversity, Tocris, Lopac, Sigma-Aldrich and other suppliers) at 2 mM stock concentration for each compound dissolved in DMSO. For confirmation studies and treatment on cells, β-lapachone, N-ethylmaleimide (NEM), iodoacetamide, H2O2, cumene hydroperoxide, and menadione were purchased from Sigma-Aldrich Chemical Co.

### In Vitro Transcription and Labeling of RNAs

In vitro transcription and labeling of RNAs was carried out using MEGAshortscript T7 transcription kit (Ambion) with the modification that 0.65 μM biotin-16-UTP (Roche) and 2.6 μM UTP were used in the reactions to produce biotinlabeled RNAs (Wan et al., 2005). Cell culture, drug treatment and preparation of cell extracts HeLa S3 cell pellets were purchased from National Cell Culture Center. Cytoplasmic extracts competent for snRNP assembly were prepared as described (Pellizzoni et al., 2002). Total cell extracts were made by resuspending cell pellets in reconstitution buffer (20 μM HEPES/KOH pH7.9, 50 μM KCl, 5 μM MgCl2, 0.2 mM EDTA, 5% glycerol) containing 50 g/ml digitonin (Calbiochem) and 1× Complete EDTA-free Protease Inhibitor Cocktail (Roche), followed by sonication. Nonidet P-40 (NP-40) was added to a final concentration of 0.01%, followed by centrifugation at 10,000 rpm for 15 minutes at 4° C. Glycerol was then added to the supernatant at a final concentration of 5%. For various drug treatments, two 10 cm plates of HeLa PV cells were treated with each drug at the indicated concentrations and times. Cells were harvested (˜90% confluent) and total cell extracts prepared using the aforementioned procedures. Protein concentrations of various extracts were determined using the Bradford protein assay (BioRad). All of the extracts for the same experiment were adjusted to the same final protein concentration

Purification of Native, RNA-Free snRNP Proteins

Native, RNA-free total snRNP proteins (TPs) were prepared from HeLa S3 cells (National Cell Culture Center) as described previously (Sumpter et al., 1992). For in vitro Sm core assembly on ³²P-labeled snRNAs in cytoplasmic extracts, the reactions were carried out at 30° C. for 1 hour using standard assembly reaction conditions (Pellizzoni, et al., 2002, Science 298: 1775-1779). Subsequently, half of the reaction mixtures were loaded onto native gels for electrophoretic mobility shift assays as described in (Pellizzoni, et al., 2002, Science 298: 1775-1779). The other half of the samples were immunoprecipitated with anti-Sm monoclonal antibody (Y12), and the immunoprecipitated RNAs were isolated and analyzed by electrophoresis on 7 mM urea/8% polyacrylamide gels.

For quantitative in vitro assembly assays on magnetic beads, cytoplasmic extracts were prepared and used for assembly on biotin-labeled snRNAs using the standard reconstitution conditions in 96-well plates. Following the reactions, Y12 antibodies immobilized onto the magnetizable Dynabeads Protein A (Dynal Biotech ASA, Oslo, Norway) in 100 μl of RSB-500 buffer (10 μM Tris/HCl pH 7.5, 500 μM NaCl, 2.5 μM MgCl.sub.2) containing 2 mg/ml heparin, 0.1% NP-40, and 0.2 U/.μl RNasin Plus RNase inhibitor (Promega, Madison, Wis.) were added to each well. Immunoprecipitations in the 96-well plates were carried out with gentle mixing at 750 rpm in a Thermomixer (Eppendorf, Germany) at 30° C. for 1 hour. The plates were subsequently transferred to a Kingfisher 96 magnetic particle processor (Thermo Labsystems, Vantaa, Finland) for automatic washing of the Dynabeads in each well with wash buffer (RSB-500, 0.1% NP-40) 5 times. After the last wash, beads bound to Y12 immunoprecipitated snRNPs were then resuspended in 120 μl of wash buffer containing 0.08 μg/ml horseradish peroxidase (HRP)-conjugated NeutrAvidin.TM. (Pierce, Rockford, Ill.). Following incubation at 30° C. for 1 hour with gentle mixing, the beads in each well were again washed five times with the Kingfisher 96 magnetic particle processor, and finally resuspended in 150 μl of SuperSignal ELISA Femto Substrate Working Solution (Pierce). The plates were transferred to a Wallac Victor2 multi-label plate reader (Perkin-Elmer, Wellesley, Mass.) for luminescence measurements at 495 nm. The resulting data were analyzed with Microsoft Excel.

High Throughput SMN Complex Activity Assay for in Vitro Assembly of snRNPs

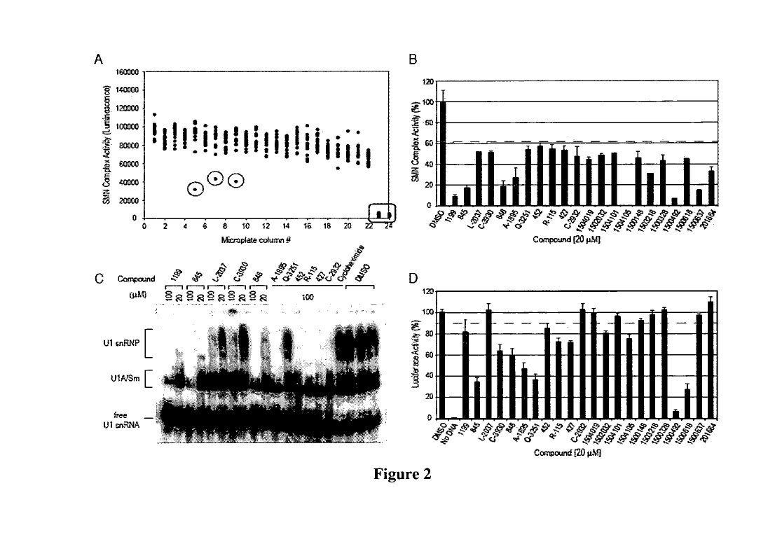

An automated and quantitative assay for in vitro snRNP assembly was developed for high throughput screening using biotinylated U4 snRNA to capture snRNPs assembled from cell extracts onto 384-well neutravidin coated microplates. The plate map for the screen is shown in FIG. 2A. Columns 1 and 2 contained controls corresponding to 100% assembly activity in the presence of DMSO. Library compounds were located in the 320 central wells, and columns 23 and 24 contained controls for nonspecific background (no HeLa cell extract added). The assay was conducted as follows: 10 l of reconstitution buffer containing 2.5 μM ATP, 0.25 g/l Escherichia coli tRNA, 0.2 U/l RNasin RNase Inhibitor (Promega) and HeLa cytoplasmic extract (4 g of total protein) was aliquoted into Reacti-Bind NeutrAvidin coated black 384-well microplates (Pierce) with a Multidrop (Thermo labsystems). Next, 51 of compounds dissolved in 80% DMSO and reconstitution buffer (final concentration 20 μM in <0.1% DMSO), and 5 l of biotin-labeled U4 snRNA (final concentration 5 nM) were added to each well using Biomek FX workstation (Beckman Coulter). The plates were centrifuged gently, and the assembly reactions were incubated for 1 hour at room temperature (RT). Subsequently, 20 l of RSB-500 buffer (10 μM Tris/HCl pH 7.5, 500 μM NaCl, 2.5 μM MgCl2) containing 2 mg/ml heparin, 0.1% NP-40, 0.2 U/l RNasin was added to each well, mixed by pipetting with the Biomek FX, and incubated for 1 hour at RT. The reaction mixtures were removed and 40 l/well of Y12 antibody (diluted 1:1000 in RSB-500 containing 0.1% NP-40, 1 mg/ml BSA) was added with a Multidrop (Thermolabsystems). After 1 hour incubation at RT, the plates were washed 10 times with RSB-500 containing 0.1% NP-40 using an ELx405 microplate washer (Bio-Tek), followed by the addition of 40 l/well of horseradish peroxidase (HRP)-conjugated AffiniPure goat anti-mouse IgG+IgM (diluted 1:10,000 in RSB-500 containing 0.1% NP-40, 1 mg/ml BSA; Jackson Laboratories) and incubation for 1 hour at RT. The plates were washed again 10 times and 40 l of SuperSignal ELISA Femto Maximum Sensitivity enhanced chemiluminescence substrate working solution (Pierce) was added into each well. Luminescence signals were measured using an EnVision Reader (PerkinElmer) with standard luminescence settings. Percentage activity from each well of the plate was calculated from the equation:

$$\frac{( S - \overline{N} )}{( \overline{S} - \overline{N} )} ⋆ 100$$

Where S is sample well signal, N is median non-specific background signal from wells in column 23, and S is median sample signal from 320 central wells.

### In Vitro Transcription and Translation Assay

In vitro transcription and translation reactions were set up in 384-well microplate using TNT Quick Coupled Transcription/Translation Systems (Promega) with luciferase DNA as a reporter. Briefly, reactions were set up in bulk (e.g., 40 l TNT quick master mix, 1 l of 1 μM methionine and 1 g of luciferase reporter DNA in a total of 50 l reaction volume), and then aliquoted at 2 l/well into 384-well microplate. Compounds dissolved in DMSO or DMSO alone (final DMSO concentration is 2.5%) were added to each well at 20 μM final concentration with Pintool (Kalypsys System). The plate was centrifuged briefly and the reactions were incubated at 30° C. for 1.5 hours. Subsequently, 20 l of Luciferase Assay Reagent (Promega) was added into each well and mixed. Luminescence signals were measured immediately using an EnVision Reader (PerkinElmer) with standard luminescence settings.

### Antibodies, Quantitative Western Blot Analysis and Indirect Immunofluorescence Microscopy

For quantitative Western blot, cell extracts containing 20 g of total proteins were mixed with NuPAGE LDS sample buffer (Invitrogen) with or without reducing agent (Invitrogen), boiled for 5 minutes, and then separated on NuPAGE 4-12% Bis-Tris gels (Invitrogen) and transferred to nitrocellulose membranes. Quantitative Western blot analysis was performed as suggested by the manufacturer (Li-Cor) and described previously (Wan et al., 2005). Mouse monoclonal antibodies, anti-SMN (62E7), anti-Gemin3 (12H12), anti-Gemin4 (17D10), anti-Gemin5 (10G11), anti-JBP1 (6G8), anti-Magoh (18G12) and anti-Sm (Y12), were used as described previously (Battle et al., 2006b; Lerner et al., 1981; Wan et al., 2005). The IRDye800 anti-mouse IgG secondary antibody (Rockland) was used at 1:5000. Indirect immunofluorescence microscopy using monoclonal antibodies 2B1 (anti-SMN) and Y12 was performed as described (Liu and Dreyfuss, 1996).

### Example 1

### Development of a High Throughput Assay for In Vitro Assembly of snRNPs

In order to perform HTS for small molecule modulators of SMN complex activity, a different snRNP assembly assay that can be performed in 384- or 1536-well plate format was developed. The scheme of this assay, illustrated in FIG. 1, consists of incubating snRNAs, which are produced and labeled with biotins by in vitro transcription, with cell extracts in the presence of ATP for Sm core assembly to occur. The reactions are performed in avidincoated microplates, which capture the RNAs onto the plate surface by avidin-biotin binding. After washing at high stringency with a buffer that spares only the highly stable Sm cores, the amount of Sm cores that assemble on the captured RNAs is determined with a monoclonal anti-Sm antibody (Y12), which is then detected with horseradish peroxidase (HRP)-conjugated secondary antibody. The HRP provides enzymatic amplification of a chemiluminescent substrate and the signal in each well is measured in a plate reader. Readings of a representative 384-well plate are shown in FIG. 2A. Each dot represents the luminescence signal from a single well, which contains a standard assembly reaction with HeLa cell extract and U4 snRNA in the presence of a compound. The ratio of signal to background (assembly reactions without cell extracts, indicated in the green box) obtained in this assay is approximately 30, the standard deviation (SD) from all the assay wells is 13.17%, and the Z′ factor is 0.54. Compounds that have significant effects can easily be identified (dots circled in red), demonstrating that this assay is sensitive and robust, and therefore suitable for high throughput screens. Subsequent technical improvements, including direct conjugation of HRP to Y12, resulted in further improved assay parameters and simplified assay procedures. Initial screening was performed on a collection of ˜5,000 bioactive compounds at a final concentration of 20 μM in triplicate. In this collection, 22 compounds strongly inhibited the activity of the SMN complex (>3SD; FIG. 2B), but none were found that significantly increased it. The inhibitory activities of these compounds were confirmed and validated in magnetic beads snRNP assembly assay (Wan et al., 2005). Further validation of the HTS method was provided using gel mobility shift assay, a standard method for studying snRNP assembly (Kleinschmidt et al., 1989; Raker et al., 1999), with both U1 and U4 snRNAs (FIG. 2C).

To assess the selectivity of the compounds and eliminate general inhibitors of ATP-dependent processes and RNA-protein interactions, a counter-screening assay was set up. For this purpose, in vitro transcription and translation of luciferase DNA was carried out in rabbit reticulocyte lysate in the presence of 20 μM compound or DMSO control, and the amount of luciferase activity produced from the translated proteins was measured. As shown in FIG. 2D, 13 out of the 22 compounds identified from the primary screen also inhibited transcription and translation by >3SD of the assay, suggesting that these are not selective inhibitors of snRNP assembly.

### Example 2

### Validation and Selectivity Screens for the Inhibitors of snRNPs Assembly

To validate the hits from the primary screen described above, the effect of the compounds was tested by gel mobility shift assay, a standard method for studying snRNP assembly. In this assay, ³²P-labeled snRNAs were incubated with cell extracts in the presence of ATP, and the resulting stable Sm cores, resolved by electrophoresis from free RNAs, were visualized by their characteristic mobility shift on native polyacrylamide gels. The reactions were incubated with either 20 or 100 μM of each compound, or with DMSO as a control. Representatives of several of these compounds are shown in FIG. 3B. This confirmed that most compounds obtained from the primary screen significantly inhibited Sm core assembly at the screening concentration of 20 μm and all of them almost completely inhibited assembly at 100 μm. Similar results were obtained for U1 and U4 snRNAs in both the high throughput assay and the gel mobility shift assay, demonstrating the generality of the effect of these compounds on snRNP assembly.

To assess whether the compounds inhibit snRNP assembly selectively and to eliminate those that inhibit other ATP-dependent processes or general RNA-protein interactions, a counter-screening assay was set up. An in vitro transcription and translation assay in rabbit reticulocyte lysate of luciferase mRNA produced from a plasmid in the presence of 20 μm compound or DMSO control, and measured the amount of luciferase activity produced from the translated proteins, was performed. As shown in FIG. 3C, 13 out of the 22 compounds identified from the primary screen also inhibited transcription and translation by >3SD of the assay, demonstrating that these are not selective inhibitors of snRNP assembly. Thus, the transcription and translation assay is an effective filter to eliminate some non-specific inhibitors.

### Example 3

### β-Lapachone Inhibits snRNP Assembly in Cell Extracts and in Living Cells

The in vivo effects of the remaining compounds on HeLa cells were tested next. Several of the compounds were extremely toxic to cells even at low concentrations and were not studied further. For the rest, extracts from cells treated for 6 hours with subtoxic concentrations of each compound were prepared and their snRNP assembly activities were measured.

This Example describes one of these, b-lapachone (3,4-dihydro-2,2-dimethyl-2H-naphthol[1,2-b]pyran-5,6-dione; FIG. 3A, labeled as L-2037), that showed a particularly strong inhibition of about 90%, but did not significantly inhibit the transcription and translation of a transfected luciferase reporter. Inhibition of snRNP assembly by >80% was observed within 30 minutes of b-lapachone treatment, indicating that its inhibition of the SMN complex is due to a direct effect of the compound. The inhibition of the activity of the SMN complex in treated cells was dependent on b-lapachone concentration with a calculated IC50 of 0.45 μM (FIG. 3B). IC50 estimated from dose-response measurements in vitro using extracts of untreated cells was ˜30 μM. Thus, b-lapachone inhibits the activity of the SMN complex both in cell extracts and in living cells, and it is much more potent in vivo than in vitro.

The persistence of the inhibition in extracts prepared after b-lapachone was washed away from the treated cells suggested that it either caused a modification of one or more components in the snRNP assembly pathway or interfered with protein-protein or protein-RNA interactions. It was first asked whether b-lapachone inhibits the interactions among the Sm proteins or their capacity to bind snRNAs. Sm cores can form spontaneously in vitro from purified Sm proteins and exogenous snRNAs without auxiliary factors. This Sm core assembly, unlike assembly in cell extracts and in vivo, is independent of the SMN complex and does not require ATP. It reflects the high propensity of the Sm proteins to form heptameric cores on many Sm site-resembling sequences, but lacks the strict specificity that the SMN complex confers towards its appropriate substrates, the snRNAs. Therefore a comparison was made of the effect of b-lapachone on snRNP assembly using either cell extracts, which contain the SMN complex or all the components required for Sm core assembly, or a purified fraction of native snRNP proteins which is highly enriched in Sm proteins but lacking the SMN complex components. As shown in FIG. 3C, both of these preparations assembled Sm cores on the U4 snRNA but not on the control U4□Sm RNA which lacks the Sm site. As expected, b-lapachone inhibited assembly in the cell extracts, which is SMN complex-directed, however, it had no effect on Sm core formation from the purified snRNP proteins. This indicates that the target of b-lapachone inhibition is not the Sm-Sm protein interactions or the Sm-snRNA binding, but rather is likely the assembly machinery, the SMN complex.

### Example 4

### β-Lapachone Inhibits the SMN Complex-Mediated Assembly of Sm Cores but not Sm-Sm or Sm-snRNA Interactions

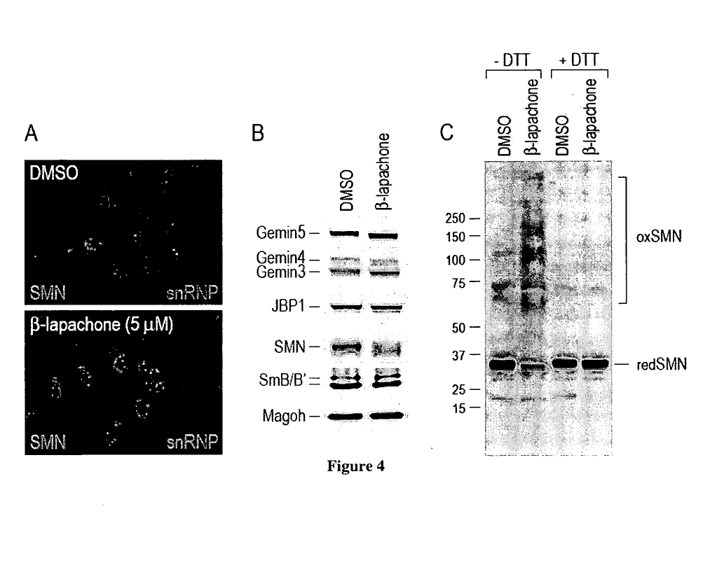

Sm cores form spontaneously in vitro from purified Sm proteins and exogenous snRNAs without auxiliary factors. This Sm core assembly, unlike assembly in cell extracts and in vivo, is independent of the SMN complex and does not require ATP. It reflects the high propensity of the Sm proteins to form heptameric cores on any Sm site-resembling sequences, but lacks the strict specificity that the SMN complex confers towards its appropriate substrates, the snRNAs (Pellizzoni et al., 2002). This assembly assay was used to determine if β-lapachone inhibits Sm core assembly from purified Sm proteins. β-lapachone was added to biotin-labeled snRNA substrates and HeLa cell extracts (containing the SMN complex and all the components required for Sm core assembly) and separately to purified, native, RNA-free snRNP proteins (enriched for Sm proteins and lacking the SMN complex components). As shown in FIG. 4D, both of these preparations assembled Sm cores on the U4 snRNA but not on the control U4 Sm RNA which lacks the Sm site. However, β-lapachone inhibited assembly in the cell extracts, but had no effect on Sm core formation from the purified snRNP proteins. This indicates that the target of β-lapachone is not the Sm-Sm interactions or the interaction of the Sm proteins with the snRNA, and it is likely the assembly machinery, the SMN complex.

### Example 5

### β-Lapachone Induces Intermolecular Disulfide Bond Formation of SMN

To further investigate the mechanism of action of b-lapachone, we asked whether it affected the cellular localization or composition of the SMN complex and of proteins involved in the snRNP biogenesis pathway. For these experiments, cells were treated with either 5 μM b-lapachone or DMSO control for 3 hours. Typically, by immunofluorescence microscopy, SMN is found throughout the cytoplasm and in the nucleus where it is concentrated in Gems. However, no detectable difference was observed in SMN signal intensity or localization between b-lapachone treated and control cells (FIG. 4A). There was also no obvious change in the amount or localization of Sm proteins, most of which are found in snRNPs and are detected by Y12 antibody (FIG. 4A).

b-lapachone is a redox-active ortho-quinone. Therefore the possibility was considered, that the inhibition of the activity of the SMN complex by b-lapachone may be due to its activity as a generator of redox cycles, a process in which the quinone is reduced and reactive oxygen species (ROS), superoxide anion (O2⁻) and hydrogen peroxide (H2O2) are produced. b-lapachone cannot directly react with sulfhydryl groups by 1,4-Michael addition since it is fully-substituted, but the ROS it generates might oxidize sulfhydryls which might then form disulfides or other cysteine modifications in protein components of the SMN complex. To test this, extracts from b-lapachone-treated cells (at 5 μM for 3 hours) or DMSO-treated control cells were prepared in gel electrophoresis sample buffer without the reducing agent dithiothreitol (DTT) to preserve potential disulfides, resolved by SDS-PAGE, and analyzed by quantitative Western blots. To simultaneously monitor different SMN complex components and associated proteins, the blots were cut into strips corresponding to the known molecular mass of the various proteins at their monomer size, and each was then probed separately for SMN, Gemin 2, 3, 4 and 5, the Sm proteins (SmB/B′, SmD1, SmD3, and SmE), or the methyltransferase JBP1 (FIG. 4B, and data not shown). Strikingly, while there was no detectable change in signal intensities of the other proteins examined, the intensity of the SMN band was consistently and significantly lower in b-lapachone treated cells prepared without DTT (˜60% decrease compared to DMSO controls; FIG. 4B). A similar decrease in the SMN band was observed on non-reducing Western blots of b-lapachone treated cell extracts. Because the decrease in monomeric-size SMN most likely resulted from ROS induced disulfide crosslinking, Western blots were performed on SMN complexes purified from b-lapachone- or DMSO-treated cell extracts prepared either with or without DTT, and probed the entire length of the membrane with anti-SMN antibody. As shown in FIG. 4C, in the absence of DTT, the band at ˜35 kDa, corresponding to monomeric SMN (redSMN), is decreased upon b-lapachone treatment and several higher molecular weight SMN-reactive forms (oxSMN), corresponding in sizes to SMN dimer and possibly larger forms are observed. When the same samples were prepared with reducing agent DTT, all the higher molecular weight SMN-containing bands disappeared and the same amount of SMN migrated at the monomer size in both b-lapachone-treated and control extracts. These findings indicate that upon b-lapachone treatment, the SMN protein experienced an oxidative environment and became crosslinked via disulfide bonds. It is noted, that even in the DMSO control, there was a detectable low level of SMN crosslinking. The b-lapachone-induced crosslinking appeared to be selective to SMN; it was not observed for the other proteins that were probed for in the same experiment, including several Gemins, the methyltransferase JBP1, Magoh and Sm proteins (FIG. 4B), as well as several other proteins tested, e.g., hnRNP A1 and the fragile X mental retardation protein FMR1.

### Example 6

### DTT Reverses β-Lapachone Inhibition of the Activity of the SMN Complex

To further determine if the inhibition of the activity of the SMN complex by ROS is related to the oxidative modification of cysteines, reflected by disulfide protein crosslinking, it was asked whether b-lapachone inhibition could be prevented by the reducing agent DTT, since DTT reversed SMN protein crosslinking (FIGS. 4C and 5D). In vitro assembly reactions were performed in the presence of 20 μM b-lapachone or DMSO control, and with or without 20 μM DTT. As shown in FIG. 6, DTT completely neutralized the inhibitory effect of b-lapachone. Furthermore, the extent of SMN oxidative crosslinking, determined by the corresponding decrease in unoxidized monomeric SMN (redSMN), is directly proportional to the inhibition of SMN complex activity. Similarly, another reductant and antioxidant N-acetyl-L-cysteine (NAC) also completely prevented the inhibition of b-lapachone, menadione and H2O2. This strongly suggests that ROS inhibit the activity of the SMN complex by causing oxidation of sulfhydryl groups of cysteines and inducing disulfide crosslinking, including in SMN itself.

### Example 7

### ROS Inhibit the SMN Complex in Vitro and in Cells

The data described in the Examples above indicate that the activity of the SMN complex is regulated by redox status and could be sensitive to other oxidative stress-inducing agents. Therefore the effect of other sources of ROS were examined. H2O2 and cumene hydroperoxide, both of which are capable of oxidizing cysteines, inhibited the activity of the SMN complex in vitro with IC50 values in the low millimolar range (FIG. 5A). Menadione (vitamin K3; 2-methyl-1,4-napthoquinone), a redox-active p-quinone, inhibited the activity of the SMN complex much more efficiently, yielding an IC50 of 25 μM (FIG. 5B). Treatment of cells with menadione at concentrations co μM only used to study oxidative stress also resulted in dose-dependent inhibition of SMN complex activity (FIG. 5C) and a corresponding degree of SMN disulfide crosslinking (FIG. 5D), similar to that observed with b-lapachone. These findings indicate that the SMN complex is susceptible to inactivation by ROS, and its activity can be modulated by the cellular redox state.

Additional direct evidence for the ROS-generating activity of the compounds in live cells was obtained by staining cells with 2′,7′-dichlorodihydrofluorescein diacetate (H2DCFDA), a nonfluorescent dye that becomes fluorescent upon exposure to ROS. Dose-dependent fluorescence was detected in cells treated with b-lapachone or menadione, indicating that these compounds generate ROS in the cells (FIG. 5E). Cells treated with DMSO or with β-lapachone alone without H2DCFDA, used as controls, showed low background-level fluorescence. A significant level of ROS was apparent at 0.5 μM β-lapachone and increased proportionally with increasing b-lapachone concentrations. The fluorescence intensity of b-lapachone-treated cells was much higher than that of menadione-treated cells at similar concentrations, indicating that their potency as inactivators of SMN complex corresponds to their ROS-generating capacity.

### Example 8

### ROS Cause SMN-SMN Disulfide Crosslinking Through at Least Two Cysteines

SMN can form homo-oligomers and it interacts with many other proteins. The apparent sizes of the ROS induced disulfide-crosslinked forms correspond to SMN dimers or larger forms (FIGS. 4C and 5D), and it was therefore asked whether SMN-SMN crosslinks occur. To determine this, constructs were generated for full-length wild-type human SMN, amino- and carboxylterminal deletion mutants, and SMN in which all eight cysteines were mutated to alanines. The corresponding proteins were produced by transcription and translation in vitro with [35S]methionine. The samples were then treated with b-lapachone to generate ROS or with DMSO as a control, and resolved by SDS-PAGE without DTT. As shown in FIG. 7B, wild-type SMN (WT) formed disulfide-crosslinked species similar in sizes to those observed in b-lapachone-treated cells and cell extracts (FIGS. 4C and 5D). This demonstrates that intermolecular SMN-SMN oxidative crosslinks occur. Although not precisely defined, sequences capable of homotypic interactions have been described in exons 6-7 and in exon 2b. However, while SMN with an amino terminal deletion that includes sequences encoded by exons 1-2b (FIG. 7C, labeled as Ex3-7) could still form a disulfide crosslinked dimer, deletion of the carboxyl terminal sequences encoded by exons 5-7 (FIG. 7C, labeled as Ex1-4) completely abolished oxidative crosslinking. Additionally, no oxidative SMN-SMN crosslinking occurred in exon 7-deleted SMN (FIG. 7B, labeled as DEx7), the predominant product of the remaining SMN2 gene in SMA patients, which is defective in oligomerization. It was concluded that SMN oligomerization, mediated by its carboxyl terminus, is required for SMN-SMN disulfide crosslinking.

### Example 9

### Cells from SMA Patients and Cells Treated with Oxidants have a Detectably Lower Capacity for snRNP Formation

### In Vitro Transcription of RNAs

Plasmids for in vitro transcription of snRNAs were generated as described (Mattaj, 1986, Cell 46: 905-911; Fischer and Luhrmann, 1990, Science 249; 786-790; Ha μM, et al., 1990, Cell 62: 569-577; Jarmolowski, et al., 1993, Embo J. 12: 223-232). For radio-labeling of RNAs, in vitro transcription was carried out in the presence of [³²P]UTP as described in Yong, et al. (2002, Embo J 21: 1188-1196). Biotin-labeled RNAs were produced according to the manufacturer's protocol (Ambion, Woodward, Tex.) with the modification that 5 μM biotin-UTP (Roche, Indianapolis, Ind.) and 2.5 μM UTP were present. All of the labeled RNAs were purified by electrophoresis on 7 mM urea/6% polyacrylamide gels, precipitated with ethanol, and resuspended in nuclease-free water. The concentrations of the biotin-labeled RNAs were determined by absorbance at 260 nm.

### Cell Lines and Cell Culture Maintenance

The maintenance of the S5 cell line, which is a chicken DT40 cell line with targeted disruption of the SMN gene, is described in Wang and Dreyfuss, 2001, J. Biol Chem 276; 9599-9605). EBV-transformed lymphoblast cell lines derived from a 6 month old SMA type I patient (GM10684) and an age- and gender-matched individual with a syndrome unrelated to SMA as a control (GM12497) were obtained from Coriell Cell Repositories and maintained in RPMI 1640 medium (Gibco BRL, La Jolla, Calif.) containing 10% fetal bovine serum (HyClone, Logan, Utah) and 1% penicillin-streptomycin (Gibco BRL). Primary fibroblast cell lines from four SMA type I patients (GM00232, GM09677, GM03813 and GM03815), one heterozygous carrier (GM03814) and two apparently healthy controls (GM08333 and GM00498) were also obtained from Coriell Cell Repositories. These cells were maintained in minimum essential medium (Gibco BRL) containing 15% fetal bovine serum, 2 μM L-glutamine and 1% penicillin-streptomycin.

Preparation of Cytoplasmic Extracts from Cultured Cells

For each sample in the assays disclosed in this Example, the same number (about 4×10⁷) of cells were harvested and washed twice with PBS. For the SMA fibroblast cell lines, cells used were at the early and the same passage stage. Cytoplasmic extracts competent for snRNP assembly were prepared as described in (Pellizzoni, et al., 2002, Science 298: 1775-1779). The protein concentrations of the various extracts were determined using the Bio-Rad protein assay (BIO RAD, Hercules, Calif.). All of the extracts were adjusted to the same final protein concentration (.about. 15 μg/ml for extracts prepared from HeLa cells, S5 cells or SMA lymphoblastoid cells, and 3 μg/ml for extracts of SMA fibroblast cells) and were quickly frozen in liquid nitrogen and stored in aliquots at −80 degree. C.

### Induction of Oxidative Stress

Two healthy cell lines (GM08333 and GM00498) obtained from Coriell Cell Repositories are treated with phosphamidon (80 μg/ml) and dieldrine (25 μM) which induce oxidative stress.

Pulse-Label Measurements of the Rate of snRNP Biogenesis in Cells

Three days after chicken S5 cells are cultured in medium containing 10 or 18 ng/ml tetracycline, equal number of cells (1×10⁷) are pulse-labeled with 25 Ci/ml [³H]uridine (Amersham, Piscataway, N.J.) for one hour. Total RNAs are isolated from 10% of the labeled cells using TRIzol reagent (Invitrogen). Total cell extracts from the remaining cells are subjected to immunoprecipitation by Y12. The immunoprecipitated RNAs are isolated by proteins K treatment followed by phenol-chloroform extraction and ethanol precipitation. Both total RNAs and Y12 immunoprecipitated RNAs are analyzed by electrophoresis on 7 mM urea/8% polyacrylamide gels. Gels are then treated with Amplify solution (Amersham) and dried for autoradiography.

Quantitation of snRNPs in Cells

To determine the overall amount of snRNPs at steady state in cells, Y12 immunoprecipitations were performed using cell extracts from 1×10⁷ S5 cells grown in the presence of either 10 or 18 ng/ml tetracycline. Y12 immunoprecipitated RNAs are isolated and radioactively labeled at the 3′ end with [5′-³²P]pCp (Perkin-Elmer) and T4 RNA ligase (New England Biolabs, Beverly, Mass.). The labeled RNAs are analyzed by electrophoresis on 7 mM urea/8% polyacrylamide gels and dried for autoradiography.

The results of the experiments presented in this Example are now described.

Development of a Quantitative Assay for snRNP Assembly

Assembled Sm cores comprised of seven-membered rings of Sm proteins, unlike the complexes of individual Sm proteins or a subset of Sm proteins with RNA, are extremely sturdy and resist dissociation even at high salt, heparin and urea (Raker, et al., 1996, Embo J. 15: 2256-2269; Ha μM, et al., 1987, Embo J. 6: 3479-3485; Jarmolowski, et al., 1993, Embo J. 12: 223-232).

Following Y12 immunoprecipitation, ³²P-labeled snRNAs on which Sm cores formed are eluted and analyzed by urea/polyacrylamide gel electrophoresis. Sm cores assembled on the wild-type snRNAs but not on the respective Sm mutants and the oxidant treated cells. A weak signal is detected for both the U1A3 mutant and the oxidant treated cells, demonstrating a low level of Sm core formation. The level of snRNP assembly is estimated by autoradiography on a phosphorimager.

The snRNP assembly process is measured by preparing snRNAs by in vitro transcription in the presence of biotin-UTP. Following in vitro assembly reactions with the biotin-labeled RNAs, immunoprecipitations of the Sm cores are carried out under stringent conditions, including high salt (500 μM NaCl) and heparin (2 mg/ml). The immunoprecipitations are carried out with anti-Sm antibodies (Y12) immobilized on magnetic beads in a 96-well plate format, which allows automatic cycles of washing and mixing of the beads on a robotic manifold. Subsequently, horseradish peroxidase (HRP)-conjugated avidin, which binds tightly to biotin, is used to recognize the biotinylated RNAs in the Y12 immunoprecipitated Sm cores. This step also serves to amplify the signals for the luminescence measurement of the HRP activity on an automatic plate reader. Readings on all Sm mutants and oxidant treated cells are close to background, demonstrating the strict dependence of assembly on an Sm site. Since U1A3 binds to the SMN complex with lower affinity than wild-type U1 and Sm cores assemble on U1A3 with slower kinetics in vivo in Xenopus oocytes, the difference could be due to the higher concentration of U1A3 RNA and longer reaction time used in this assay. Notably, the experimental variation in independent experiments is typically less than 5% of the signal. The assay disclosed herein is more sensitive, much less labor-intensive than previous methods and amendable to high-throughput automation.

Deficiency in snRNP Assembly in Cell Extracts Obtained from GM08333 and GM00498 Cells Treated with an Oxidant and SMA Patients

In light of the findings described herein, specifically that SMN protein levels parallel the capacity of snRNP assembly in HeLa cells and in chicken S5 cells, cells from SMA patients and GM08333 and GM00498 cells treated with an oxidant are analyzed to determine if they are also deficient in snRNP assembly activity. A lymphoblastoid cell line derived from a type I SMA patient (GM10684) was examined using a similarly established cell line from an age- and gender-matched individual as a control (GM12497). Immunoblotting demonstrated that SMN in the patient cells and in GM08333 and GM00498 cells treated with an oxidant was considerably reduced whereas the levels of Gemins2-5 and SmB/B′ are similar to the control. Compared to the control, snRNP assembly activity of the patient cell extract and GM08333 and GM00498 cell extract derived from cells treated with an oxidant was 48% of the control, respectively, in three independent experiments. This assembly capacity demonstrates a biochemical deficiency in cells of an SMA patient and GM08333 and GM00498 cells treated with an oxidant.

The data obtained from these experiments demonstrates that the assembly activities in GM08333 and GM00498 cells treated with an oxidant is considerably lower than those in the lymphoblastoid cells. The assembly assay revealed that extracts from GM08333 and GM00498 cells treated with an oxidant had approximately 40% of the assembly activity compared to the corresponding controls, consistent with the degree of reduction of SMN protein in these cells.

The SMN complex, under the conditions utilized herein, required for snRNP assembly. Specifically, complete, or nearly complete, removal or inhibition of the SMN complex results in the inhibition of Sm core assembly in vitro. Using an in vitro assay developed for the quantitative measurement of the Sm core assembly process in cell extracts, the data disclosed herein demonstrate that there is a linear correlation between the amount of SMN present in the cell extract and the amount of Sm cores that can be formed on specific RNA substrates, indicating that the amount of SMN determines the capacity for Sm core assembly. This data demonstrates a direct correlation between the degree of reduction of SMN protein levels in cells obtained from SMA patients and GM08333 and GM00498 cells treated with an oxidant. However, an understanding of the molecular consequences of the reduced levels of SMN in patients' cells has been lacking. The present invention demonstrates that snRNP assembly is impaired in cells of SMA patients and GM08333 and GM00498 cells treated with an oxidant. Consistent with the reduced activity observed in extracts of cells with low SMN, the rate of production of snRNPs is these cells is profoundly reduced.

### Example 10

### Antioxidants Administration to SMA I Patients Elevate snRNP Assembly

The following four different multi antioxidant formulations are prepared for treating or abrogating SMA:

(1) Vitamin A 1 mg, Vitamin C 90 mg, Vitamin E 25 mg, Selenium 55 μg.

(2) Vitamin A 1.2 mg, Vitamin C 500 mg, manganese 1.5 mg

(3) Zeaxanthin 80 mg, isoflavone 500 mg, Vitamin C 100 mg

(4) Citric acid 1000 mg, Vitamin A 1.2 mg, stilbenoid 500 mg, lycopene 30 mg

All formulations are prepared as 10 ml solutions for oral administration. Eight patients are divided to 4 groups according to the 4 formulations. Each group consumes 2 dosages a day during breakfast and dinner. Cells are obtained from each individual 8 patients prior to the treatment are compared to cells obtained from the same patients a week, 4 weeks, 8 weeks, 12 weeks, and 24 weeks into the treatment. Cells are analyzed for their capacity for snRNP formation (for materials and methods refer to example 9).

The results obtained from this study show that a steady increase of 5% in snRNP assembly is accomplished every 4 weeks after the initiation of the antioxidant treatment.

What is claimed is:

1. A method of treating a spinal muscular atrophy in a subject, comprising the step of administering to said subject a compound which inhibits SMN protein oxidation, thereby treating a spinal muscular atrophy in a subject.

2. The method of claim 1, wherein said spinal muscular atrophy is Werdnig-Hoffmann disease

3. The method of claim 1, wherein said spinal muscular atrophy is SMA type II.

4. The method of claim 1, wherein said spinal muscular atrophy is Kugelberg-Welander disease.

5. The method of claim 1, wherein said spinal muscular atrophy is progressive spinobulbar muscular atrophy.

6. The method of claim 1, wherein said spinal muscular atrophy is Congenital SMA with arthrogryposis.

7. The method of claim 1, wherein said compound which inhibits SMN protein oxidation is an antioxidant.

8. The method of claim 7, wherein said antioxidant is a vitamin.

9. The method of claim 7, wherein said antioxidant is a vitamin cofactor or a vitamin mineral.

10. The method of claim 7, wherein said antioxidant is a hormone.

11. The method of claim 7, wherein said antioxidant is a carotenoid.

12. The method of claim 7, wherein said antioxidant is a flavonoid.

13. The method of claim 7, wherein said antioxidant is a phenolic acid or a phenolic acid ester.

14. The method of claim 7, wherein said antioxidant is vitamin C.

15. The method of claim 7, wherein said antioxidant is vitamin E.

16. The method of claim 7, wherein said antioxidant is ebselen.

17. The method of claim 7, wherein said antioxidant is idebenone.

18. The method of claim 1, wherein said compound which inhibits SMN protein oxidation is a compound which reverses SMN oxidation.

19. The method of claim 18, wherein said compound which reverses SMN oxidation is a reducing agent.

20. The method of claim 1, wherein said compound which inhibits SMN protein oxidation is a compound which boosts catalase enzymatic activity.

21. The method of claim 1, wherein said compound which inhibits SMN protein oxidation is a compound which boosts glutathione enzymatic activity.

22. The method of claim 1, wherein said compound which inhibits SMN protein oxidation is a compound which boosts peroxidase enzymatic activity.

23. The method of claim 1, wherein said compound which inhibits SMN protein oxidation is a compound which boosts superoxide dismutase (SOD) enzymatic activity.

24. A method of abrogating spinal muscular atrophy in a subject, comprising the step of administering to said subject a compound which inhibits SMN protein oxidation, thereby abrogating spinal muscular atrophy in a subject.

25. The method of claim 24, wherein said spinal muscular atrophy is Werdnig-Hoffmann disease

26. The method of claim 24, wherein said spinal muscular atrophy is SMA type II.

27. The method of claim 24, wherein said spinal muscular atrophy is Kugelberg-Welander disease.

28. The method of claim 24, wherein said spinal muscular atrophy is progressive spinobulbar muscular atrophy.

29. The method of claim 24, wherein said spinal muscular atrophy is Congenital SMA with arthrogryposis.

30. The method of claim 24, wherein said compound which inhibits SMN protein oxidation is an antioxidant.

31. The method of claim 30, wherein said antioxidant is a vitamin.

32. The method of claim 30, wherein said antioxidant is a vitamin cofactor or a vitamin mineral.

33. The method of claim 30, wherein said antioxidant is a hormone.

34. The method of claim 30, wherein said antioxidant is a carotenoid.

35. The method of claim 30, wherein said antioxidant is a flavonoid.

36. The method of claim 30, wherein said antioxidant is a phenolic acid or a phenolic acid ester.

37. The method of claim 30, wherein said antioxidant is vitamin C.

38. The method of claim 30, wherein said antioxidant is vitamin E.

39. The method of claim 30, wherein said antioxidant is ebselen.

40. The method of claim 30, wherein said antioxidant is idebenone.

41. The method of claim 24, wherein said compound which inhibits SMN protein oxidation is a compound which reverses SMN oxidation.

42. The method of claim 41, wherein said compound which reverses SMN oxidation is a reducing agent.

43. The method of claim 24, wherein said compound which inhibits SMN protein oxidation is a compound which boosts catalase enzymatic activity.

44. The method of claim 24, wherein said compound which inhibits SMN protein oxidation is a compound which boosts glutathione enzymatic activity.

45. The method of claim 24, wherein said compound which inhibits SMN protein oxidation is a compound which boosts peroxidase enzymatic activity.

46. The method of claim 24, wherein said compound which inhibits SMN protein oxidation is a compound which boosts superoxide dismutase (SOD) enzymatic activity.

47. A method of preventing a spinal muscular atrophy in a subject, comprising the step of administering to said subject a compound which inhibits SMN protein oxidation, thereby preventing a spinal muscular atrophy in a subject.

48. The method of claim 47, wherein said compound which inhibits SMN protein oxidation is an antioxidant.

49. The method of claim 48, wherein said antioxidant is a vitamin.

50. The method of claim 48, wherein said antioxidant is a vitamin cofactor or a vitamin mineral.

51. The method of claim 48, wherein said antioxidant is a hormone.

52. The method of claim 48, wherein said antioxidant is a carotenoid.

53. The method of claim 48, wherein said antioxidant is a flavonoid.

54. The method of claim 48, wherein said antioxidant is a phenolic acid or a phenolic acid ester.

55. The method of claim 48, wherein said antioxidant is vitamin C.

56. The method of claim 48, wherein said antioxidant is vitamin E.

57. The method of claim 48, wherein said antioxidant is ebselen.

58. The method of claim 48, wherein said antioxidant is idebenone.

59. The method of claim 47, wherein said compound which inhibits SMN protein oxidation is a compound which reverses SMN oxidation.

60. The method of claim 59, wherein said compound which reverses SMN oxidation is a reducing agent.

61. The method of claim 47, wherein said compound which inhibits SMN protein oxidation is a compound which boosts catalase enzymatic activity.

62. The method of claim 47, wherein said compound which inhibits SMN protein oxidation is a compound which boosts glutathione enzymatic activity.

63. The method of claim 47, wherein said compound which inhibits SMN protein oxidation is a compound which boosts peroxidase enzymatic activity.

64. The method of claim 47, wherein said compound which inhibits SMN protein oxidation is a compound which boosts superoxide dismutase (SOD) enzymatic activity.

65. A method of protecting an SMN protein in a subject, comprising the step of administering to said subject a compound which inhibits SMN protein oxidation, thereby protecting an SMN protein in a subject.

66. The method of claim 65, wherein said compound which inhibits SMN protein oxidation is an antioxidant.

67. The method of claim 65, wherein said antioxidant is a vitamin.

68. The method of claim 66, wherein said antioxidant is a vitamin cofactor or a vitamin mineral.

69. The method of claim 66, wherein said antioxidant is a hormone.

70. The method of claim 66, wherein said antioxidant is a carotenoid.

71. The method of claim 66, wherein said antioxidant is a flavonoid.

72. The method of claim 66, wherein said antioxidant is a phenolic acid or a phenolic acid ester.

73. The method of claim 66, wherein said antioxidant is vitamin C.

74. The method of claim 66, wherein said antioxidant is vitamin E.

75. The method of claim 66, wherein said antioxidant is ebselen.

76. The method of claim 66, wherein said antioxidant is idebenone.

77. The method of claim 66, wherein said protecting an SMN protein comprises protecting an SMN protein oxidized.

78. The method of claim 65, wherein said compound which inhibits SMN protein oxidation is a compound which reverses SMN oxidation.

79. The method of claim 78, wherein said compound which reverses SMN oxidation is a reducing agent.

80. The method of claim 65, wherein said compound which inhibits SMN protein oxidation is a compound which boosts catalase enzymatic activity.

81. The method of claim 65, wherein said compound which inhibits SMN protein oxidation is a compound which boosts glutathione enzymatic activity.

82. The method of claim 65, wherein said compound which inhibits SMN protein oxidation is a compound which boosts peroxidase enzymatic activity.

83. The method of claim 65, wherein said compound which inhibits SMN protein oxidation is a compound which boosts superoxide dismutase (SOD) enzymatic activity.

84. A method of protecting the generation of a spliceosome in a subject, comprising the step of administering to said subject a compound which inhibits SMN protein oxidation, thereby protecting the generation of a spliceosome in a subject.

85. The method of claim 84, wherein said compound which inhibits SMN protein oxidation is an antioxidant.

86. The method of claim 85, wherein said antioxidant is a vitamin.

87. The method of claim 85, wherein said antioxidant is a vitamin cofactor or a vitamin mineral.

88. The method of claim 85, wherein said antioxidant is a hormone.

89. The method of claim 85, wherein said antioxidant is a carotenoid.

90. The method of claim 85, wherein said antioxidant is a flavonoid.

91. The method of claim 85, wherein said antioxidant is a phenolic acid or a phenolic acid ester.

92. The method of claim 85, wherein said antioxidant is vitamin C.

93. The method of claim 85, wherein said antioxidant is vitamin E.

94. The method of claim 85, wherein said antioxidant is ebselen.

95. The method of claim 85, wherein said antioxidant is idebenone.

96. The method of claim 84, wherein said compound which inhibits SMN protein oxidation is a compound which reverses SMN oxidation.

97. The method of claim 96, wherein said compound which reverses SMN oxidation is a reducing agent.

98. The method of claim 84, wherein said compound which inhibits SMN protein oxidation is a compound which boosts catalase enzymatic activity.

99. The method of claim 84, wherein said compound which inhibits SMN protein oxidation is a compound which boosts glutathione enzymatic activity.

100. The method of claim 84, wherein said compound which inhibits SMN protein oxidation is a compound which boosts peroxidase enzymatic activity.

101. The method of claim 84, wherein said compound which inhibits SMN protein oxidation is a compound which boosts superoxide dismutase (SOD) enzymatic activity.

102. A method of protecting the generation of small nuclear ribonucleoproteins (snRNPs) in a subject, comprising the step of administering to said subject a compound which inhibits SMN protein oxidation, thereby protecting the generation of snRNPs in a subject.

103. The method of claim 102, wherein said compound which inhibits SMN protein oxidation is an antioxidant.

104. The method of claim 103, wherein said antioxidant is a vitamin.

105. The method of claim 103, wherein said antioxidant is a vitamin cofactor or a vitamin mineral.

106. The method of claim 103, wherein said antioxidant is a hormone.

107. The method of claim 103, wherein said antioxidant is a carotenoid.

108. The method of claim 103, wherein said antioxidant is a flavonoid.

109. The method of claim 103, wherein said antioxidant is a phenolic acid or a phenolic acid ester.

110. The method of claim 103, wherein said antioxidant is vitamin C.

111. The method of claim 103, wherein said antioxidant is vitamin E.

112. The method of claim 103, wherein said antioxidant is ebselen.

113. The method of claim 103, wherein said antioxidant is idebenone.

114. The method of claim 102, wherein said compound which inhibits SMN protein oxidation is a compound which reverses SMN oxidation.

115. The method of claim 114, wherein said compound which reverses SMN oxidation is a reducing agent.

116. The method of claim 102, wherein said compound which inhibits SMN protein oxidation is a compound which boosts catalase enzymatic activity.

117. The method of claim 102, wherein said compound which inhibits SMN protein oxidation is a compound which boosts glutathione enzymatic activity.

118. The method of claim 102, wherein said compound which inhibits SMN protein oxidation is a compound which boosts peroxidase enzymatic activity.

119. The method of claim 102, wherein said compound which inhibits SMN protein oxidation is a compound which boosts superoxide dismutase (SOD) enzymatic activity.

120. A method of treating a disease mediated by a deficient spliceosome in a subject, comprising the step of administering to said subject a compound which inhibits SMN protein oxidation, thereby treating a disease mediated by a deficient spliceosome in a subject.

121. The method of claim 120, wherein said disease mediated by a deficient spliceosome is β-thalassemia.

122. The method of claim 120, wherein said disease mediated by a deficient spliceosome is severe combined immunodeficiency (SCID)

123. The method of claim 120, wherein said disease mediated by a deficient spliceosome is Metachromatic leukodystrophy.

124. The method of claim 120, wherein said disease mediated by a deficient spliceosome is Menkes Disease.

125. The method of claim 120, wherein said disease mediated by a deficient spliceosome is Multiple Sclerosis.

126. The method of claim 120, wherein said disease mediated by a deficient spliceosome is Spinal Muscular Atrophy.

127. The method of claim 120, wherein said disease mediated by a deficient spliceosome is Adenosine deaminase deficiency.

128. The method of claim 120, wherein said disease mediated by a deficient spliceosome is Cerebrotendinous xanthomatosis (CTX).

129. The method of claim 120, wherein said disease mediated by a deficient spliceosome is Sandhoff disease.

130. The method of claim 120, wherein said disease mediated by a deficient spliceosome is Marfan syndrome.

131. The method of claim 120, wherein said disease mediated by a deficient spliceosome is Breast cancer.

132. The method of claim 120, wherein said disease mediated by a deficient spliceosome is ovarian cancer.

133. The method of claim 120, wherein said disease mediated by a deficient spliceosome is Neurofibromatosis type I.

134. The method of claim 120, wherein said disease mediated by a deficient spliceosome is acute intermittent porphyria.

135. The method of claim 120, wherein said disease mediated by a deficient spliceosome is Thrombasthenia of Glanzmann and Naegeli.

136. The method of claim 120, wherein said compound which inhibits SMN protein oxidation is an antioxidant.

137. The method of claim 136, wherein said antioxidant is a vitamin.

138. The method of claim 136, wherein said antioxidant is a vitamin cofactor or a vitamin mineral.

139. The method of claim 136, wherein said antioxidant is a hormone.

140. The method of claim 136, wherein said antioxidant is a carotenoid.

141. The method of claim 136, wherein said antioxidant is a flavonoid.

142. The method of claim 136, wherein said antioxidant is a phenolic acid or a phenolic acid ester.

143. The method of claim 136, wherein said antioxidant is vitamin C.

144. The method of claim 136, wherein said antioxidant is vitamin E.

145. The method of claim 136, wherein said antioxidant is ebselen.

146. The method of claim 136, wherein said antioxidant is idebenone.

147. The method of claim 120, wherein said compound which inhibits SMN protein oxidation is a compound which reverses SMN oxidation.

148. The method of claim 147, wherein said compound which reverses SMN oxidation is a reducing agent.

149. The method of claim 120, wherein said compound which inhibits SMN protein oxidation is a compound which boosts catalase enzymatic activity.

150. The method of claim 120, wherein said compound which inhibits SMN protein oxidation is a compound which boosts glutathione enzymatic activity.

151. The method of claim 120, wherein said compound which inhibits SMN protein oxidation is a compound which boosts peroxidase enzymatic activity.

152. The method of claim 120, wherein said compound which inhibits SMN protein oxidation is a compound which boosts superoxide dismutase (SOD) enzymatic activity.

153. A method of protecting an SMN protein in a subject at risk of developing SMA, comprising the step of administering to said subject a compound which inhibits SMN protein oxidation, thereby protecting an SMN protein in a subject at risk.

154. The method of claim 153, wherein said subject at risk of developing SMA comprises a subject lacking a single copy of SMN1.

155. The method of claim 154, wherein said subject at risk of developing SMA comprises a subject having a single functional copy of SMN1.

156. The method of claim 155, wherein said subject having a single functional copy of SMN1 comprises a subject having a non-functional copy of SMN1.

157. The method of claim 155, wherein said non-functional copy of SMN1 comprises a mutated copy of SMN1.

158. The method of claim 153, wherein said compound which inhibits SMN protein oxidation is an antioxidant.

159. The method of claim 158, wherein said antioxidant is a vitamin.

160. The method of claim 158, wherein said antioxidant is a vitamin cofactor or a vitamin mineral.

161. The method of claim 158, wherein said antioxidant is a hormone.

162. The method of claim 158, wherein said antioxidant is a carotenoid.

163. The method of claim 158, wherein said antioxidant is a flavonoid.

164. The method of claim 158, wherein said antioxidant is a phenolic acid or a phenolic acid ester.

165. The method of claim 158, wherein said antioxidant is vitamin C.

166. The method of claim 158, wherein said antioxidant is vitamin E.

167. The method of claim 158, wherein said antioxidant is ebselen.

168. The method of claim 158, wherein said antioxidant is idebenone.

169. The method of claim 153, wherein said compound which inhibits SMN protein oxidation is a compound which reverses SMN oxidation.

170. The method of claim 169, wherein said compound which reverses SMN oxidation is a reducing agent.

171. The method of claim 153, wherein said compound which inhibits SMN protein oxidation is a compound which boosts catalase enzymatic activity.

172. The method of claim 153, wherein said compound which inhibits SMN protein oxidation is a compound which boosts glutathione enzymatic activity.

173. The method of claim 153, wherein said compound which inhibits SMN protein oxidation is a compound which boosts peroxidase enzymatic activity.

174. The method of claim 153, wherein said compound which inhibits SMN protein oxidation is a compound which boosts superoxide dismutase (SOD) enzymatic activity.
